(19) United States
(12) Patent Application Publication
(10) Pub. No.: US 2010/0210692 A1
(43) Pub. Date: Aug. 19, 2010

(54) METHODS OF TREATMENT USING SIRT MODULATORS AND COMPOSITIONS CONTAINING SIRT1 MODULATORS

(76) Inventors: Stephen R. Farmer, Newton, MA (US); Hong Wang, Malden, MA (US); Li Qiang, New York, NY (US)

Correspondence Address: LANDO & ANASTASI, LLP;E2023, One Main Street, Suite 1100, Cambridge, MA, 02142, US

(21) Appl. No.: 12/593,082

(22) PCT Filed: Mar. 28, 2008
(86) PCT No.: PCT/US08/58738

§ 371 Date: Apr. 14, 2010

Related U.S. Application Data
(60) Provisional application No. 60/920,517, filed on Mar. 28, 2007.
(60) Provisional application No. 60/934,764, filed on Jun. 15, 2007.

(51) Int. Cl. A61K 31/4439; A61K 31/403; A61K 31/426; A61K 31/422; A61K 31/44; A61P 3/00; A61P 9/00; A61P 35/00; A61P 29/00

(52) U.S. Cl. 514/342; 514/411; 514/369; 514/374; 514/352

(57) ABSTRACT

Methods of treatment using SIR1 modulators and compositions containing SIRT1 modulators are described. Combinations of a SIRT1 modulator and a second agent, and uses of such combinations, are also described.

### CROSS-REFERENCE TO RELATED APPLICATIONS

This application claims priority to U.S. application Ser. No. 60/920,517, filed on Mar. 28, 2007 and U.S. application Ser. No. 60/934,764, filed on Jun. 15, 2007. The disclosures of the prior applications are considered part of (and are incorporated by reference in) the disclosure of this application.

### FEDERALLY SPONSORED RESEARCH

This invention was made with Government Support under Contract Nos. DK051586 and DK058825 awarded by the National Institutes of Health. The Government has certain rights in this invention.

### BACKGROUND

An important class of HDACs is the NAD⁺-dependent sirtuins, or class III HDACs. The most prominent human family member, SIRT1 (Q96E136), has been shown to regulate transcriptional repression of mammalian target genes that are either already basally expressed or to regulate transcriptional repression of an integrated Gal4-fusion reporter plasmid.

### SUMMARY

Applicants have discovered that inhibitors of SIRT1 can be used to treat various disorders, for example, metabolic disorders, neoplastic disorders such as cancer, dyslipidemia, arteriosclerosis, inflammatory disorders, cardiovascular disorders, and ischemia. In some embodiments, the disorders can be treated with a combination of a SIRT1 inhibitor and an agonist of PPARγ. Applicants have also discovered that an activator of SIRT1 can be used, for example,alone or in combination with another therapeutic agent, such as a modulator of PPARγ, in the treatment of cancer by inhibition of angiogenesis.

In one aspect, the invention features a method of treating a disorder in a subject by administering to the subject a SIRT1 inhibitor. In some preferred embodiments, the method also includes administering to the subject a modulator of PPARγ, for example, a PPARγ agonist.

In one aspect, the invention includes a method of treating a disorder in a subject, the method comprising administering a SIRT1 inhibitor and a PPARγ agonist to the subject.

In some embodiments, the disorder is a metabolic disorder (e.g., as diabetes, obesity, or metabolic syndrome), a neoplastic disorder such as cancer, dyslipidemia, arteriosclerosis (e.g., atherosclerosis), inflammation, a cardiovascular disorder, or ischemia.

In some embodiments, the PPARγ agonist is a thiazolidinedione (TZD). In some embodiments, the TZD comprises rosiglitazone, pioglitazone, troglitazone, or ciglitazone.

In some embodiments, the PPARγ agonist is a non-thiazolidinedione (non-TZD). In some embodiments, the non-TZD comprises GW1929.

In some embodiments, the SIRT1 inhibitor is a small molecule inhibitor, e.g., an organic molecule having a molecular weight of less than about 1000 daltons, e.g., less than about 500 daltons,

In some embodiments, the SIRT1 inhibitor is splitomycin or nicotinamide.

In some embodiments, the the SIRT1 inhibitor is a compound of formula (I)

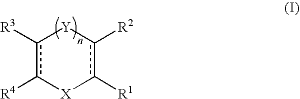

wherein

R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵; or R¹ is H, S-alkyl, or S-aryl, and R² is amidoalkyl wherein the nitrogen is substituted with alkyl, aryl, or arylalkyl, each of which is optionally further substituted with alkyl, halo, hydroxy, or alkoxy;

R³ and R⁴, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which are optionally substituted with 1-5 R⁶; each of R⁵ and R⁶ is, independently, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, alkoxycarbonyl, C₁-C₁₀ thioalkoxycarbonyl, hydrazinocarbonyl, C₁-C₆ alkyl hydrazinocarbonyl, C₁-C₆ dialkyl hydrazinocarbonyl, hydroxyaminocarbonyl; alkoxyaminocarbonyl; or one of R⁵ or R⁶ and R⁷ form a cyclic moiety containing 4-6 carbons, 1-3 nitrogens, 0-2 oxygens and 0-2 sulfurs, which are optionally substituted with oxo or C₁-C₆ alkyl;
X is NR⁷, O, or S; Y is NR⁷ , O or S;

[symbol] represent optional double bonds;

each of R⁷ and R⁷ is, independently, hydrogen, C₁-C₆ alkyl, C₇-C₁₂ arylalkyl, C₇-C₁₂ heteroarylalkyl; or Rand one of R⁵ or R⁶ form a cyclic moiety containing 4-6 carbons, 1-3 nitrogens, 0-2 oxygens and 0-2 sulfurs, which are optionally substituted with oxo or C₁-C₆ alkyl; and

n is 0 or 1.

In some embodiments, R¹ and R², together with the carbons to which they are attached, farm C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵.

In some embodiments, R¹ and R², together with e carbons to which they are attached, form C₅-C₁₀ cycloalkenyl.

In some embodiments, R¹ and R² are substituted with R⁵.

In some embodiments, R⁵ is, C₁-C₆ alkyl substituted with a substituent or amino carbonyl, substituted with a substituent.

In some embodiments, the substituent is an amino substituent, or aminocarbonyl.

In some embodiments, R³ and R⁴, together with the carbons to which they are attached, form C₆-C₁₀ aryl.

In some embodiments, R³ and R⁴ are substituted with R⁶.

In some embodiments, R⁶ is halo or C₁-C₆ alkyl.

In some embodiments, n is 0.

In some embodiments, X is NR⁷.

In some embodiments n is 0 and X is NR⁷.

In some embodiments, the compound of formula (I) has the formula (X) below:

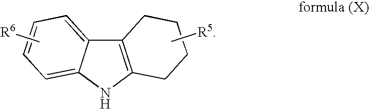

In some embodiments, R⁶ is halo or C₁-C₆ alkyl.

In some embodiments, R⁵ is aminocarbonyl.

In some embodiments, the compound of formula (I) has the formula (XI) below:

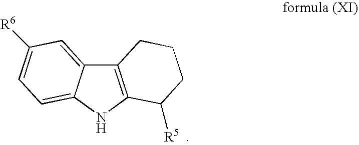

In some embodiments, R⁶ is halo or alkyl.

In some embodiments, R⁵ is aminocarbonyl,

In some embodiments, R⁶ is halo or alkyl and wherein R⁵ is aminocarbonyl.

In some embodiments, the compound is 6-Chloro-2,3,4,9-tetrahydro-1H-carbazole-1-carboxylic acid amide.

In some embodiments, the compound comprises greater than a 60% enantiomeric excess of the enantiomer having an optical rotation of −14.1 (c=0.33 DOM).

In some embodiments, the compound comprises greater than a 90% enantiomeric excess of the enantiomer having an optical rotation of −14.1 (c=0.33 DCM).

Exemplary SIRT1 inhibitors include nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem 278:52773. Analogs and derivatives of these compounds can also be used.

In certain embodiments, the natural products guttiferone G (1) and hyperforin (2) as well as the synthetic aristoforin (3) are used as inhibitors of human SIRT1. Hyperforin is one of the principal constituents identified in St John's wort. Hyperforin is a prenylated phloroglucinol. The structure of hyperforin is shown below:

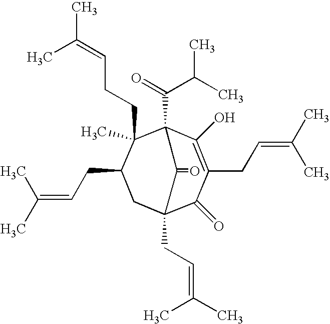

Guttiferone is a prenylated benzophenone. Guttiferone A is found in both Garcinia livingstonei T. Anders. (Gereau and Lovett 2678), originally collected in the Mufindi District of Iringa Region of Tanzania in December of 1988, and Symphonia globulifera L.f., originally collected in the Ndakan Gorilla Study Area of the Central African Republic in March 1988 (Fay 8278). Both species are members of the Clusiaceae. The structure of guttiferone is shown below:

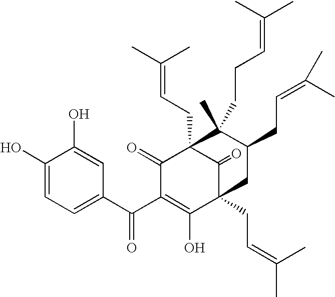

In other certain preferred embodiments, the SIRT1 inhibitors are tetrahydrocarbazole compounds. Nayagam et al., (SIRT1 modulating compounds from high-throughput screening as anti-inflammatory and insulin-sensitizing agents, J. Biomol. Screen. 2006, 11, 959-967), incorporated by reference in its entirety herein, describe tetrahydrocarbazole compounds.

US Published Application No. 2006-0111435, incorporated by reference in its entirety herein, lists a number of sirtuin-inhibitory compounds, for example:

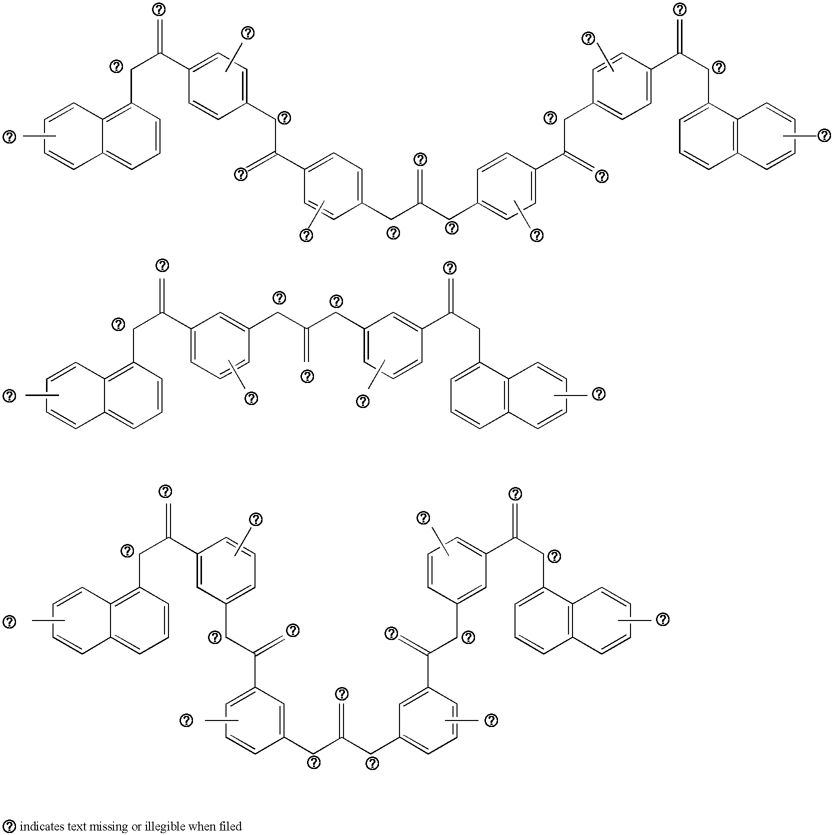

wherein, independently for each occurrence, L represents O, NR, or S; R represents H, alkyl, aryl, aralkyl, or heteroaralkyl; R′ represents H, halogen, NO.sub.2, SR, SO.sub.3, OR, NR.sub.2, alkyl, aryl, or carboxy; a represents an integer from 1 to 7 inclusively; and b represents an integer from 1 to 4 inclusively.

US Published Application No. 2007-0043050, incorporated by reference in its entirety herein, describes sirtuin-modulating compounds. Sirtuin-modulating compounds can be as below, or a salt thereof:

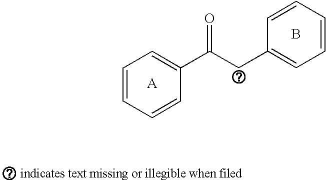

Ring A is optionally substituted, fused to another ring or both; and Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is fused to an aryl ring and is optionally substituted by one or more additional groups. Optionally the sirtuin-modulating compound can be of the formula below, or a salt thereof:

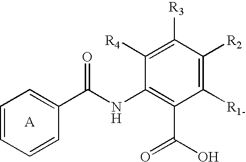

Ring A is optionally substituted; R.sub.1, R.sub.2, R.sub.3 and R.sub.4 are independently selected from the group consisting of —H, halogen, —OR.sub.5, —CN, —CO.sub.2R.sub.5, —OCOR.sub.5, —OCO.sub.2R.sub.5, —C(O)NR.sub.5R.sub.6, —OC(O)NR.sub.5R.sub.6, —C(O)R.sub.5, —COR.sub.5, —SR.sub.5, —OSO.sub.3H, —S(O).sub.nR.sub.5, —S(O).sub.nOR.sub.5, —S(O).sub.nNR.sub.5R.sub.6, —NR.sub.5R.sub.6, —NR.sub.5C(O)OR.sub.6, —NR.sub.5C(O)R.sub.6 and —NO.sub.2; R.sub.5 and R.sub.6 are independently —H, a substituted or unsubstituted alkyl group, a substituted or unsubstituted aryl group or a substituted or unsubstituted heterocyclic group; and n is 1 or 2.

Any one or more of the compounds listed in US Published Application No, 2007-0043050, US Published Application No, 2007-0037827, US Published Application No, 2007-0037865, US Published Application No, 2006-0276393, and US Published Application No. 2006-0229265 all of which are incorporated by reference in their entireties herein, are suitable for use in the invention.

In some embodiments, the SIRT1 inhibitor is an antibody that specifically binds to SIRT1.

In some embodiments, the SIRT1 inhibitor is an interfering RNA. Exemplary interfering RNA sequences include shRNA sequences, for example one of the following sequences: CTTGTACGACGAAGACGAC; GGCCACGGATAGGTCCATAT; or CATAGACACGCTGGAACAG. In some embodiments, the interfering RNA is a single stranded RNA. In some embodiments, the interfering RNA is an siRNA.

In some embodiments, the SIRT1 inhibitor and PPARγ agonist are co-administered.

In some embodiments, the SIRT1 inhibitor and PPARγ agonist are administered sequentially.

In some embodiments, the SIRT1 inhibitor and PPARγ agonist are administered in a single dosage form.

In some embodiments, the amount of SIRT1 inhibitor and PPARγ agonist are administered in an amount to provide a synergistic effect.

In some embodiments, the amount of SIRT1 inhibitor required to achieve a therapeutic effect, when co-administered with a PPARγ agonist, is less than about 85% of the amount of SIRT1 inhibitor required to achieve the therapeutic effect when administered in the absence of the PPARγ agonist (e.g., less than about 80%, less than about 75%, less than about 70%, less than about 65%, less than about 60%, less than about 55%, or less than about 50%).

In some embodiments, the amount of the PPARγ agonist required to achieve a therapeutic effect, when co-administered with SIRT1 inhibitor, is less than about 85% of the amount of the PPARγ agonist to achieve the therapeutic effect when administered in the absence of the SIRT1 inhibitor (e.g., less than about 80%, less than about 75%, less than about 70%, less than about 65%, less than about 60%, less than about 55%, or less than about 50%).

In one aspect, the invention features a composition a pharmaceutical composition) comprising a SIRT1 inhibitor and a PPARγ agonist.

The composition can e.g., be administered to a subject, e.g., a subject that has or is at risk for a disorder, nple, a metabolic disorder, a neoplastic disorder such as cancer, dyslipidemia, arteriosclerosis, inflammation, a cardiovascular disorder, or ischemia.

In one embodiment, the composition comprises an oral dosage formulation.

In one embodiment the oral dosage formulation is a tablet, a capsule, or a powder for oral suspension.

In one embodiment, the composition further comprises pharmaceutically acceptable excipient.

In one embodiment, the composition comprises from about 1 mg to about 500 mg of the SIRT1 inhibitor.

In one embodiment, the composition comprises from about 1 mg to about 500 mg of the PPARγ agonist.

In some embodiments, the PPARγ agonist is a thiazolidinedione (TZD). In some embodiments, the TZD comprises rosiglitazone, pioglitazone, troglitazone, or ciglitazone.

In some embodiments, the PPARγ agonist is a non-thiazolidinedione (non-TZD). In some embodiments, the non-TZD comprises GW1929.

In some embodiments, the SIRT1 inhibitor is a small molecule inhibitor, e.g., an organic molecule having a molecular weight of less than about 1000 daltons, e.g., less than about 500 daltons.

In some embodiments, the SIRT1 inhibitor is splitomycin or nicotinamide.

In some embodiments, the the SIRT1 inhibitor is a compound of formula (I)

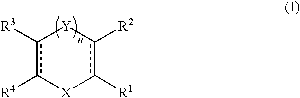

wherein,

R¹ and R², together with the carbons to which they arc attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵; or R¹ is H, S-alkyl, or S-aryl, and R² is amidoalkyl wherein the nitrogen is substituted with alkyl, aryl, or arylalkyl, each of which is optionally further substituted with alkyl, halo, hydroxy, or alkoxy;

R³ and R⁴, together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which are optionally substituted with 1-5 R⁶; each of R⁵ and R⁶ is, independently, halo, hydroxy, C₁-C₁₀ alkyl, C₁-C₆ haloalkyl, C₁-C₁₀ alkoxy, C₁-C₆ haloalkoxy, C₆-C₁₀ aryl, C₅-C₁₀ heteroaryl, C₇-C₁₂ aralkyl, C₇-C₁₂ heteroaralkyl, C₃-C₈ heterocyclyl, C₂-C₁₂ alkenyl, C₂-C₁₂ alkynyl, C₅-C₁₀ cycloalkenyl, C₅-C₁₀ heterocycloalkenyl, carboxy, carboxylate, cyano, nitro, amino, C₁-C₆ alkyl amino, C₁-C₆ dialkyl amino, mercapto, SO₃H, sulfate, S(O)NH₂, S(O)₂NH₂, phosphate, C₁-C₄ alkylenedioxy, oxo, acyl, aminocarbonyl, C₁-C₆ alkyl aminocarbonyl, C₁-C₆ dialkyl aminocarbonyl, C₁-C₁₀ alkoxycarbonyl, thioalkoxycarbonyl, hydrazinocarbonyl, C₁-C₆ alkyl hydrazinocarbonyl, C₁-C₆ dialkyl hydrazinocarbonyl, hydroxyaminocarbonyl; alkoxyaminocarbonyl; or one of R⁵ or R⁶ and R⁷ form a cyclic moiety containing 4-6 carbons, 1-3 nitrogens, 0-2 oxygens and 0-2 sulfurs, which are optionally substituted with oxo or C₁-C₆ alkyl;

X is NR⁷, O, or S; Y is NR⁷, O or S;

[symbol] represent optional double bonds;

each of R⁷ and R^{7′} is, independently, hydrogen, C₁-C₆ alkyl, C₇-C₁₂ arylalkyl, C₇-C₁₂ heteroarylalkyl; or R⁷ and one of R⁵ or R⁶ form a cyclic moiety containing 4-6 carbons, 1-3 nitrogens, 0-2 oxygens and 0-2 sulfurs, which are optionally substituted with oxo or C₁-C₆ alkyl; and

n is 0 or 1.

In some embodiments, R¹ and R², together with the carbons to which they are attached, form C₅-C₁₀ cycloalkyl, C₅-C₁₀ heterocyclyl, C₅-C₁₀ cycloalkenyl, C₅-C₁₀ heterocycloalkenyl, C₆-C₁₀ aryl, or C₆-C₁₀ heteroaryl, each of which may be optionally substituted with 1-5 R⁵.

In some embodiments, R¹ and R², together with the carbons to which they are attached, form C₅-C₁₀ cycloalkenyl.

In some embodiments, R¹ and P² are substituted with R⁵.

In some embodiments, R⁵ is, C₁-C₆ alkyl substituted with a substituted amino carbonyl, substituted with a substituent.

In some embodiments, the substituent is an amino substituent, or aminocarbonyl.

In some embodiments, R³ and R⁴, together with the carbons to which they are attached, form C₆-C₁₀ aryl.

In some embodiments, R³ and R⁴ are substituted with R⁶.

In some embodiments, R⁶ is halo or C₁-C₆ alkyl.

In some embodiments, n is 0.

In some embodiments, X is NR⁷.

In some embodiments n is 0 and X is NR⁷.

In some embodiments, the compound of formula (I) has the formula (X) below:

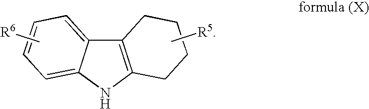

In some embodiments, R⁶ is halo or C₁-C₆ alkyl.

In some embodiments, R⁵ is aminocarbonyl.

In some embodiments, the compound of formula (I) has the formula (XI) below:

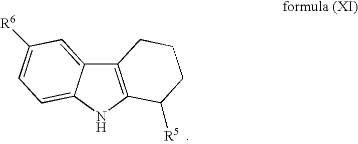

In some embodiments, R⁶ is halo or alkyl,

In some embodiments, R⁵ is aminocarbonyl.

In some embodiments, R⁶ is halo or alkyl and wherein R⁵ is aminocarbonyl.

In some embodiments, the compound is 6-Chloro-2,3,4,9-tetrahydro-1H-carbazole-1-carboxylic acid amide.

In some embodiments, the compound comprises greater than a 60% enantiomeric excess of the enantiomer having an optical rotation of −14.1 (c=0.33 DCM).

In some embodiments, the compound comprises greater than a 90% enantiomeric excess of the enantiomer having an optical rotation of −14.1 (c=0.33 DCM).

Exemplary SIRT1 inhibitors include nicotinamide (NAM), suranim; NF023 (a G-protein antagonist); NF279 purinergic receptor antagonist); Trolox (6-hydroxy-2,5,7,8,tetramethylchroman-2-carboxylic acid); (−)-epigallocatechin (hydroxy on sites 3,5,3′,4′,5′); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; (3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) J. Biol. Chem. 276:38837, Dedalov et al. (2001) PNAS 98:15113 and Hirao et al. (2003) J. Biol. Chem 278:52773. Analogs and derivatives of these compounds can also be used.

In certain embodiments, the natural products guttiferone G (1) and hyperforin (2) as well as the synthetic aristoforin (3) are used as inhibitors of human SIRT1. Hyperforin is one of the principal constituents identified in St John's wort. Hyperforin is a prenylated phloroglucinol. The structure of hyperforin is shown below:

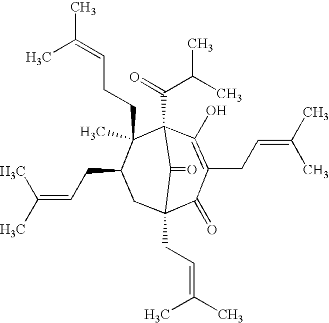

Guttiferone is a prenylated benzophenone. Guttiferone A is found in both Garcinia livingstonei T. Anders, (Gereau and Lovett 2678 originally collected in the Mufindi District of Iringa Region of Tanzania in December of 1988, and Symphonia globulifera L.f., originally collected in the Ndakan Gorilla Study Area of the Central African Republic in March 1988 (Fay 8278). Both species are members of the Clusiaceae. The structure of guttiferone is shown below:

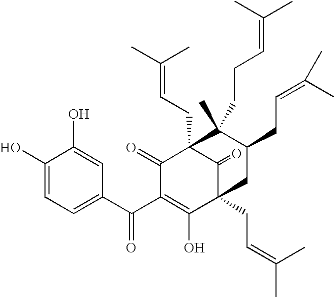

In other certain preferred embodiments, the SIRT1 inhibitors are tetrahydrocarbazole compounds. Nayagam et al., (SIRT1 modulating compounds from high-throughput screening as anti-inflammatory and insulin-sensitizing agents, J. Biomol. Screen. 2006, 11, 959-967), incorporated bey reference in its entirety herein, describe tetrahydrocarbazole compounds.

US Published Application No. 2006-0111435, incorporated by reference in its entirety herein, lists a number of sirtuin-inhibitory compounds, for example:

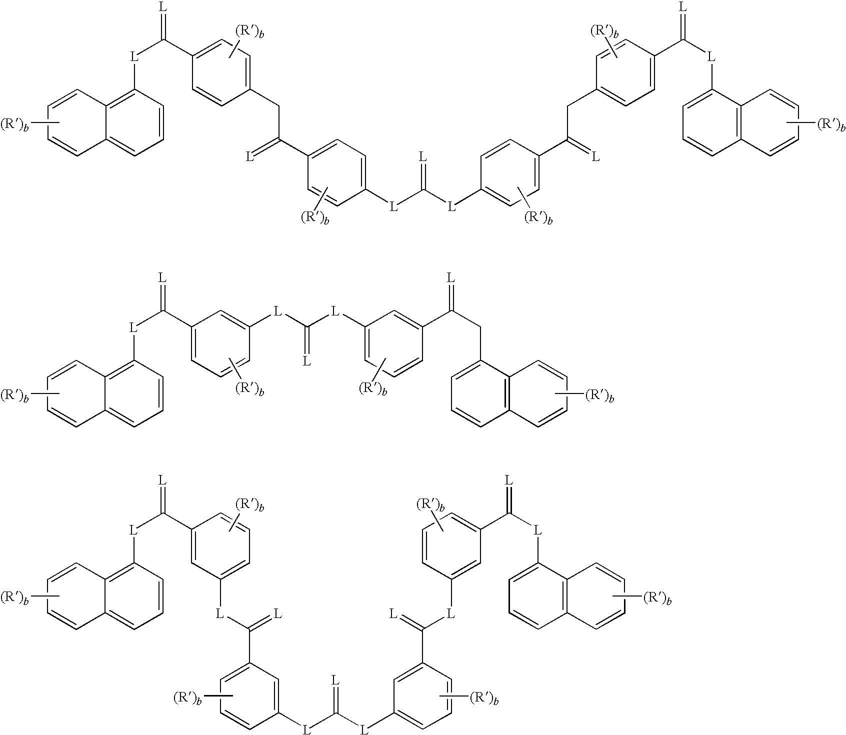

wherein, independently for each occurrence, L represents O, NR, or S; R represents H, alkyl, aryl, aralkyl, or heteroaralkyl; R′ represents H, halogen, NO.sub.2, SR, SO.sub.3, OR, NR.sub.2, alkyl, aryl, or carboxy; a represents an integer from 1 to 7 inclusively; and b represents an integer from 1 to 4 inclusively.

US Published Application No. 2007-0043050, incorporated by reference in its entirety herein, describes sirtuin-modulating compounds. Sirtuin-modulating compounds can be as below, or a salt thereof:

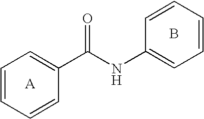

Ring A is optionally substituted, fused to another ring or both; and Ring B is substituted with at least one carboxy, substituted or unsubstituted arylcarboxamine, substituted or unsubstituted aralkylcarboxamine, substituted or unsubstituted heteroaryl group, substituted or unsubstituted heterocyclylcarbonylethenyl, or polycyclic aryl group or is Based to an aryl ring and is optionally substituted by one or more additional groups. Optionally, the sirtuin-modulating, compound can be of the formula below, or a salt thereof:

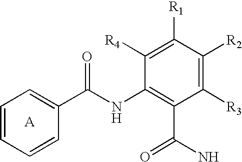

Ring A is optionally substituted; R.sub.1, R.sub.2, R.sub.3 and R.subA are independently selected from the group consisting of —H, halogen, —OR.sub.5, —CN, —CO.sub.2R.sub.5, —OCOR.sub.5, —OCO.sub.2R.sub.5, —C(O)NR.sub.5R.sub.6, —OC(O)NR.sub.5R.sub.6, —C(O)R.sub.5, —COR.sub.5, —SR.sub.5, —OSO.sub.3H, —S(O).sub.nR.sub.5, —S(O).sub.nOR.sub.5, —S(O).sub.nNR.sub.5R.sub.6, —NR.sub.5R.sub.6, —NR.sub.5C(O)OR.sub.6, —NR.sub.5C(0)R.sub.6 and —NO.sub.2; R.sub.5 and R.sub.6 are independently —H, a substituted or unsubstituted alkyl group, a substituted or unsubstituted aryl group or a substituted or unsubstituted heterocyclic group; and n is 1 or 2.

Any one or more of the compounds listed in US Published Application No. 2007-0043050, US Published Application No, 2007-0037827, US Published Application No. 2007-0037865, US Published Application No. n 2006-0276393, and US Published Application No. 2006-0229265 all of which are incorporated by reference in their entireties herein, are suitable for use in the invention.

In some embodiments, the SIRT1 inhibitor is an antibody that specifically binds to SIRT1.

In some embodiments, the SIRT1 inhibitor is an interfering RNA. Exemplary interfering RNA sequences include shRNA sequences, for example one of the following sequences: CTTGTACGACGAAGACGAC; GGCCACGGATAGGTCCATAT; or CATAGACACGCTGGAACAG. In some embodiments, the interfering RNA is a single stranded RNA. In some embodiments, the interfering RNA is an siRNA,

In some aspects, the disclosure provides compounds and combinations described herein a pharmaceutical composition) for use in therapy.

In other aspects, the disclosure describes the use of compounds described herein (e.g., in a pharmaceutical composition) for the preparation of a medicament for the treatment of a disorder (e.g., a metabolic disorder, a neoplastic disorder such as cancer, dyslipidemia, arteriosclerosis, inflammation, a cardiovascular disorder, or ischemia), in a subject (e.g., human).

In other aspects, the disclosure describes the use of a SIRT1 inhibitor and a PPARγ agonist for the preparation of a medicament for the treatment of a disorder (e.g., a metabolic disorder, a neoplastic disorder such as cancer, dyslipidemia, arteriosclerosis, inflammation, a cardiovascular disorder, or ischemia), in a subject (e.g., human).

In one aspect, the invention features a method of treating cancer in a subject by administering to the subject a SIRT1 activator. In some preferred embodiments, the method also includes administering to the subject a modulator of PPARγ, for example, a PPARγ antagonist.

In one aspect, the invention features a method of inhibiting or decreasing angiogenesis in a subject by administering to the subject a SIRT1 activator. In some preferred embodiments, the method also includes administering to the subject a modulator of PPARγ, for example, a PPARγ antagonist.

In another aspect, the invention features a composition that contains a SIRT1 activator and a PPARγ antagonist.

In other aspects, the disclosure describes the use of a composition described herein (e.g., a composition containing a SIRT1 activator and a PPARγ antagonist) for the preparation of a medicament for the treatment of a disorder (e.g., cancer or angiogenesis), in a subject (e.g., human).

In other aspects,the disclosure describes of a SIRT1 activator and a PPARγ antagonist for preparation of medicament for the treatment of a disorder (e.g., cancer or angiogenesis), in a subject (e.g., human).

In some aspects, the invention features a method of treating a disorder in a subject by administering to the subject a SIRT1 inhibitor and sitagliptin, metformin, an angiotensin II receptor blocker, retinoid, simvastatin, a statin, a sulfonylurea, a natural or synthetic RXR ligand, a dipeptidyl peptidase IV (DPP IV) inhibitor, insulin, or a Retinoid X Receptor (RXR) agonist. The method can further include administering a PPARγ agonist.

In another aspect, the invention features a composition (e.g., a pharmaceutical composition) that contains a SIRT1 inhibitor and sitagliptin, metformin, an angiotensin II receptor blocker, retinoid, simvastatin, a statin, a sulfonylurea, a natural or synthetic RXR ligand, a dipeptidyl peptidase IV (DPP IV) inhibitor, insulin, or a Retinoid X Receptor (RXR) agonist. The composition can further include a PPARγ agonist.

As used herein, the term “decrease” refers to a decrease relative to a standard. For example, a SIRT1 activator can decrease angiogenesis, e.g., relative to a standard. A suitable standard can be, e.g., the amount of angiogenesis that was measured prior to the first administration of a treatment. A treatment decreases angiogenesis if the amount of angiogenesis after treatment is less than the amount before treatment.

As used herein, the term “increase” refers to an increase relative to a standard. For example, a SIRT1 inhibitor can increase expression of FGF21, e.g., relative to a standard. A suitable standard can be, e.g., the amount of FGF21 that was present prior to the first administration of a treatment. A treatment increases expression of FGF21 if the amount of FGF21 after treatment is greater than the amount before treatment.

A subject can be “at risk” for a disorder, for example, if the subject has a factor that has been identified with an increased likelihood (e.g., as compared to a subject without the factor or the average for a cohort of subjects) of developing the disorder. For example, genetic predisposition or an inherited genetic mutation can place a subject at risk for developing a disorder associated with that factor, e.g., a BRCA1 mutation can make a subject at risk for developing breast cancer. As a further example, the presence of obesity is a factor that places a subject at risk for developing metabolic syndrome.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl). The terms “arylalkyl” or “aralkyl” refer to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups.

The term “alkylene” refers to a divalent alkyl, e.g., —CH₂—, —CH₂CH₂—, and —CH₂CH₂CH₂—.

The term “alkenyl”” refers to a straight or branched hydrocarbon chain containing 2-12 carbon atoms and having one or more double bonds. Examples of alkenyl groups include, but are not limited to, allyl, propenyl, 2-butenyl, 3-hexenyl and 3-octenyl groups. One of the double bond carbons may optionally be the point of attachment of the alkenyl substituent. The term “alkynyl” refers to a straight or branched hydrocarbon chain containing 2-12 carbon atoms and characterized in having one or more triple bonds. Examples of alkynyl groups include, but are not limited to, ethynyl, propargyl, and 3-hexynyl. One of the triple bond carbons may optionally be the point of attachment of the alkynyl substituent.

The terms “alkylamino” and “dialkylamino” refer to —NH(alkyl) and —NH(alkyl)₂ radicals respectively. The term “aralkylamino” refers to a —NH(aralkyl) radical. The term alkylaminoalkyl refers to a (alkyl)NH-alkyl-radical; the term dialkylaminoalkyl refers to a (alkyl)₂N-alkyl-radical The term “alkoxy” refers to an —O-alkyl radical. The term “mercapto” refers to an SH radical. The term “thioalkoxy” refers to an —S-alkyl radical. The term thioaryloxy refers to an —S aryl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic,or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl' refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “oxo” refers to an oxygen atom, which forms a carbonyl when attached to carbon, an N-oxide when attached to nitrogen, and a sulfoxide or sulfone when attached to sulfur.

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

The terms “aminocarbonyl,” alkoxycarbonyl,” hydrazinocarbonyl, and hydroxyaminocarbonyl refer to the radicals —C(O)NH₂, —C(O)O(alkyl), —C(O)NH₂NH₂, and —C(O)NH₂NH₂, respectively.

The term “amindo”refers to a —NHC(O)-radical, wherein N is the point of attachment.

The term “substituents” refers to a group “substituted” on an alkyl, cycloalkyl, alkenyl, alkynyl, heterocyclyl, heterocycloalkenyl, cycloalkenyl, aryl, or heteroaryl group at any atom of that group. Any atom can be substituted. Suitable substituents include, without limitation, alkyl (e.g., C1, C2, C3, C4, C5, C6, C7, C8, C9, C10, C11, C12 straight or branched chain alkyl), cycloalkyl, haloalkyl perfluoroalkyl such as CF₃), aryl heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkyl amino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di alkyl, aralkyl, heteroaralkyl, and combinations thereof). In one aspect, the substituents on a group are independently any one single, or any subset of the aforementioned substituents. In another aspect, a substituent may itself be substituted with any one of the above substituents,

All cited patents, patent applications, and references are hereby incorporated by reference in their entireties. In the case of conflict, the present application controls.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1: Nutrients selectively increase secretion of HMW complexes of adiponectin. 3T3-L1 cells were cultured normally until day 4 of differentiation, at which stage medium was changed to either standard DMEM containing 5 mM glucose (C and D) or glucose-free DMEM (A and B) and in each case the DMEM was supplemented with 10% FBS. Cultures A and B were also exposed to the indicated concentration of D-glucose, whilst C and D were exposed to 20mM lactate. On Day 7, the medium and cell layer were harvested and subjected to either reducing (A, C) or non-reducing (B, D) SDS-PAGE as outlined in materials and methods.

FIG. 2: Perturbation of SIRT1 activity affects secretion of HMW forms of adiponectin, 3T3-L1 adipocytes (4 day) were exposed to either resveratrol (50 μM) or (5 mM) in standard DMEM containing 10% EBS for 2 days. The cells were then cultured in fresh DMEM overnight at which time the medium and total cell layer were harvested for western blot analysis of intra- and extra-cellular proteins on reducing (A) and non-reducing (B) SDS-PAGE as outlined in materials and methods.

FIG. 3: Perturbation of PPARγ activity affects secretion of adiponectin, 3T3-L1 adipocytes were exposed to troglitazone (5 μM) or a PPARγ antagonist (10 μM T0070907) in standard DMEM containing 10% FBS for 3 days at which time the medium and total cell layer were harvested for western blot analysis of intra- and extra-cellular proteins on reducing (A) and non-reducing (B) SDS-PAGE as outlined in materials and methods.

FIG. 4: Inhibition of SIRT1 expression enhances secretion of HMW adiponectin. 3T3-L1 preadipocytes expressing a control vector or SIRT1 siRNA were differentiated for the indicated days and medium (Extracellular) as well as total cell layer (Intracellular) was harvested for western blot analysis of proteins on reducing (A) or non-reducing (B) SDS-PAGE employing antibodies to the following proteins: SIRT1, PPARγ, C/EBPα, adiponectin, adipsin and aP2/FABP4.

FIG. 5: Activation of PPARγ in Swiss 3T3 fibroblasts induces Ero1-Lα expression as well as adipogenesis. Swiss 3T3 fibroblasts expressing wild type PPARγ or a control vector were induced to differentiate by exposure to a differentiation cocktail of dexamethasone, isobutylmethylxanthine and insulin in 10% FBS as outlined in Materials and Methods (Con). Some cultures were also exposed to 5 μM troglitazone (Trog) or 10 μM of a PPARγ antagonist, T0070907 (antag) along with the differentiation cocktail. At day 6, cells were harvested for analysis of individual mRNAs as indicated using RT-PCR.

FIG. 6: SIRT1 regulates expression of the ER oxidoreductase, Ero1-Lα. (A) 3T3-L1 preadipocytes were differentiated for the indicated days and total cell RNA was extracted for RT-PCR analysis of mRNAs corresponding to PPARγ, C/EBPα, adiponectin, Ero1-Lα, FABP4/aP2 and GAPDH. 3T3-L1 preadipocytes expressing a control vector (−) or a SIRT1 siRNA (+) were differentiated for 8 days for RT-PCR analysis of mRNAs (B) or for the indicated days for western blot analysis of intracellular proteins (C). Control and SIRT1 siRNA expressing 3T3-L1 preadipocytes were differentiated in the absence (Con) or presence of either an PPARγ antagonist (Antag, 10 μM T0070907) or PPARγ agonist (Trog, 5 μM troglitazone) and medium (extracellular) and total cell layer (intracellular) was harvested for western blot analysis of indicated proteins (D).

FIG. 7: Knockdown of Ero1-Lα expression in 3T3-L1adipocytes inhibits secretion of adiponectin. 3T3-L1 adipocytes were transiently transfected with siRNAs corresponding to Ero1-Lα (1, 2 and 3) or a control siRNA as described in Materials and Methods. On day 6, total cell extracts (intracellular) and medium (extracellular) were harvested and subjected to Western blot analysis of Ero1-Lα, adiponectin, actin and adipsin.

FIG. 8: Ectopic expression of Ero1-Lα enhances the secretion of adiponectin in 3T3 adipocytes. (A) Swiss 3T3 fibroblasts expressing either a WT-PPARγ (WT) or the mutant F372A-PPARγ (F) were induced to differentiate as described in materials and methods and total cell extracts (Int) as well as medium (Ext) were harvested at day 5 for western blot analysis of indicated proteins. (B) and (C). Swiss F372A PPARγ cells expressing either a pREV-TET Ero1-Lα retrovirus (F-Ero) or a control vector (F-Con) were induced to differentiate in the presence (+) or absence (−) of tetracycline for 5 days as described in materials and methods. At this stage, medium (Ext) and total cell protein (Int) were harvested and subjected to reducing (B) or non-reducing without heat (C) SDS-PAGE followed by western blot analysis using antibodies against the indicated proteins. In C, WT proteins from (A) above were also analyzed as a control.

FIG. 9: Ectopic expression of Ero1-Lα attenuates the inhibitory effects of resveratrol on adiponectin secretion. Swiss F372A PPARγ cells expressing either a pREV-TET Ero1-Lα retrovirus (F-Ero) or a control vector (F-Con) were induced to differentiate by exposure to differentiation medium and 5 μM troglitazone in the presence (+T) or absence (−T) of tetracycline for 5 days as described in materials and methods. At day 4, cells were treated with or without 50 μM resveratrol for 2 days and medium (Ext) or total cell protein (Int) were harvested for western blot analysis of indicated proteins.

FIG. 10: Establishment of conditions for analysis of intro- and extra-cellular forms of adiponectin in 3T3-L1 adipocytes. Total cell layer (intracellular) medium (extracellular) proteins (40 μg) from cultures of 3T3-L1 adipocytes (7 days) were subjected to western blot analysis under different conditions as outlined in materials and methods. Before adopting this methodology in the present studies, we needed to establish conditions that are appropriate for analyzing the complexity of both intracellular as well as extracellular adiponectin produced by 3T3-L1 adipocytes. The data show that separation of intracellular and extracellular proteins on SDS-PAGE under stringent reducing conditions (100 mM DTT) and denaturation of the proteins by high temperature dissociates any higher ordered complexes, giving rise to predominantly adiponectin monomers migrating at 30 kD (lanes 2 and 4). Eliminating heat, but still under reducing conditions (100 mM DTT) results in migration of a 66 kD trimer (lanes 1 and 3). Further analysis reveals that preservation of the higher-ordered complexity of adiponectin requires SDS-PAGE performed under non-reducing conditions without heating the samples (lanes 6 and 8). It is noteworthy that adiponectin complexes secreted from the adipocytes consist primarily of multimers (HMW) and hexamers (MMW) of the 30 kD polypeptide (lane 6). The intracellular complexes consist of abundant levels of the trimer, in addition to some HMW and hexamer complexes (lane 8).

FIG. 11: Total cellular proteins were collected at day 5 and subjected to western blot analysis with the indicated antibodies.

FIG. 12. (A): Identification of two groups of PPARγ-responsive genes: Group 1 (including adiponectin) is responsive whereas Group 2 (including Ero1 and FGF21) is completely unresponsive to troglitazone activation of EF-PPARγ or F-PPARγ. Swiss fibroblasts (C) and Swiss-PPARγ (WT, EF, F) cells were cultured until confluent, after 2 days, they were exposed to DEX, MIX, insulin, with or without 5 μM troglitazone for 5 days. Total RNA of cells was isolated using Trizol Reagent (Invitrogen) and subjected to RT PCR analysis as described in materials and methods. (B): Troglitazone selectively enhances expression of the Group 2 PPARγ-responsive genes during the differentiation of Swiss fibroblasts expressing WT-PPARγ, Swiss WT-PPARγ cells were cultured in 10% FBS until they reached confluence. After 2 days post-confluence, cells were induced to differentiate by exposure to DEX, MIX, insulin, and 10% FBS with or without troglitazone. At day 0, 1 6, 7 of differentiation, cells were harvested for RT-PCR analysis as described rials and methods.

FIG. 13: Select group 2 PPARγ-responsive genes arc transiently induced during the initial phase of adipogenesis in white 3I3-L1 preadipocytes (A) and immortalized primary brown preadipocytes (B). A. 3T3-L1 white preadipocytes were cultured in 10% calf serum until they reached confluence. At 2 days post-confluence, cells were induced to differentiate by exposure to DEX, MIX, insulin and 10% PBS, At the indicated days of differentiation, cells were harvested for RT-PCR analysis as described in materials and methods. B. Immortalized brown preadipocytes were grown to confluence in differentiation medium composed of DMEM containing 10% FBS supplemented with 20 nM insulin and 1 nM 3,3′,5-triiodo-4-thyronine (T3). At 2 days post-confluence, cells were induced to differentiate by exposure to DEX, MIX, insulin, 0.125 mM indomethacin and 10% FBS. At the indicated days of differentiation, cells were harvested for RT-PCR analysis as described in materials and methods.

FIG. 14: The differential response of WT-PPARγ versus EF-PPARγ to select PPARγ ligands and antagonists. (A) Swiss-PPARγ (WT and EF) cells were cultured until confluent, after 2 days, they were exposed to DEX, MIX, insulin, with or without the following PPARγ ligands: FMOC-leu (15 μM), 15δ-PGD2 (7 μM), troglitazone (5 μM), rosiglitazone (10 μM) and GW1929 (10 μM). (B) WT-PPARγ cells were differentiated as in A by exposure to DEX, MIX and insulin in the presence or absence of troglitazone with or without either T0070907 (10 μM) or GW9662 (10 μM) (PPARγ antagonists). (C) WT-PPARγ and EF-PPARγ cells were induced to differentiate with DEX, MIX, insulin and the indicated doses of troglitazone. In (A), (B) and (C), total RNA of cells was isolated at day 5 using Trizol Reagent (Invitrogen) and subjected to RT-PCR analysis of the indicated Group 1 and Group 2 genes as described in materials and methods.

FIG. 15: PPARγ directly activates the FGF21 gene. (A). Troglitazone activates FGF21 gene expression in absence of ongoing protein synthesis. WT-PPARγ cells were induced to differentiate with DEX, MIX and insulin for 5 days at which time troglitazone (5 μM) was added in the presence or absence of cycloheximide (5 μg/ml) for the indicated times. Cells were then harvested for extraction of RNA followed by RT-PCR analysis of FGF21 and FABP4/aP2 mRNAs as described in materials and methods. (B). Reporter assays were performed in control Swiss fibroblasts following transfection of individual FGF21 luciferase plasmids (−1500, −1300 and −500 bp fragments) along with a PPARγ (pBabe-PPARγ) or control (pBabe-Puro) expression plasmid and a renilla based pGL3 reporter as control in the presence, or absence of the potent PPARγ ligand GW1929. The scheme above shows the presence of putative PPREs in the upstream region of the that have the following DR-1 sequences: 1, AGACCAAGGAGCA; 2, AGACCCAAGGCCC;3,TGGCCTGTGGCCA;4,TGAGCACAAGGCCC;3, AGTTCCAGGGCCA. (C). Reporter assays were also performed in Swiss fibroblasts stably expressing a WT-PPARγ or a pBabe-puro empty vector (control cells) following transfection of the FGF21 promoter reporter plasmids along with the renilla control vector in the presence or absence of GW1929. In both assays (B) and (C), a set of cells were also transfected with a reporter plasmid consisting of the PPRE from the aP2 gene upstream of luciferase within pGL3 (DR-1). Transcriptional activity of each of the fragments of the FGF21 gene promoter is shown as the ratio of luciferase to renilla activity (Luc/Ren) as described in materials and methods.

FIG. 16: Suppression of SIRT1 by expression of a corresponding SIRT1 siRNA selectively enhances the expression of Group 2 PPARγ-responsive genes during the differentiation of 3T3-L1 preadipocytes. Control and SIRT1 siRNA cells were cultured in 10% calf serum until they reached confluence. After 2 days post-confluence, cells were induced to differentiate by exposure to DEX, MIX, insulin, and 10% FBS. At the indicated days of differentiation, cells were harvested for western blot analysis (A) and RT-PCR analysis (B) of the indicated gene products as described in materials and methods.

FIG. 17: (A): Troglitazone selectively activates expression of Group 2 genes in 3T3-L1 adipocytes. 3T3-L1 preadipocytes were differentiated using standard conditions and at day 2, day 4 and day 6 the differentiating cells were exposed to 5 μM troglitazone for 2 days. Untreated and treated cells were harvested for analysis of select mRNAs using RT-PCR as described in materials and methods. (B): Knockdown of SIRT1 in 3T3-L1 preadipocytes enhances the expression of FGF21 in response to exposure to troglitazone. Control and SIRT1 knockdown 3T3-L1 preadipocytes were differentiated for 4 days at which time cells were exposed to the indicated doses of troglitazone for 2 days. Total RNA of cells was isolated at day 6 and subjected to RT-PCR analysis for analysis of the indicated Group 1 and Group 2 genes as described in materials and methods. (C). Model proposing interplay between PPARγ and SIRT1 in controlling adipocyte function, PPARγ functions to regulate adipocyte formation and function. Endogenous ligands activate PPARγ requiring participation of both helix 7 and 12 to orchestrate adipogenesis. In mature adipocytes, SIRT1 mediates hormonal and nutrient control of select PPARγ target genes that are involved in controlling metabolism by suppressing the action of endogenous ligands. The thiazolidinedione (TZD) family of synthetic PPARγ ligands can overcome the suppressive effects of SIRT1 acting through helix 7 as well as helix 12 to induce the metabolic genes.

FIG. 18: An illustration of fold difference in expression of select mRNAs in WT-PPARγ cells relative to EF-PPARγ cells differentiated in the absence of troglitazone. Total RNA of WT-PPARγ and EF-PPARγ cells at day 5 of differentiation were isolated using Trizol Reagent (Invitrogen) and microarray analysis was performed as described in materials and methods. Light grey corresponds to a high level of expression in WT vs EF cells (positive numbers on the log¹⁰ scale), whereas darker grey represents mRNAs expressed at low abundance in WT vs EF cells (negative numbers in the log¹⁰ scale).

FIG. 19: Exposure of EF-PPARγ or F-PPARγ cells to troglitazone is incapable of inducing secretion of adiponectin or expression of Ero1-Lα, but does induce adiponectin synthesis. Swiss-PPARγ (WT, EF, F, E) cells were cultured until confluent, after 2 days they were exposed to DEX, MIX, insulin with 5 μM troglitazone and total intra- and extra-cellular proteins were collected at day 5 for western blot analysis of PPARγ, C/EBPα, FABP4/aP2, adiponectin, Ero1-Lα as described in materials and methods.

FIG. 20: SIRT1 inhibitors (E2 and E3) potentiate the activity of troglitazone in inducing FGF21 expression in fibroblasts that ectopically express PPARγ.

### DETAILED DESCRIPTION

Combination Therapy

Combination of SIRT1 Modulator with a PPARγ Modulator

The present disclosure provides, inter alia, the use of a SIRT1 modulator e:g, a inhibitor) combination with a second agent, such as a PPARγ modulator (e.g., agonist). Inhibitors of SIRT1 can be used to treat various disorders, for example, metabolic disorders, neoplastic disorders such as cancer, dyslipidemia, arteriosclerosis, inflammation, cardiovascular disorders, and ischemia. In some embodiments, the disorders can be treated with a combination of a SIRT1 inhibitor and an agonist of PPARγ.

In other embodiments, an activator of SIRT1 can be used, for example, alone or in combination with another therapeutic agent, such as a modulator of PPARγ (e.g., PPARγ antagonist), in the treatment of cancer, e.g., by inhibition of angiogenesis.

The combination of agents described herein can have additive or synergistic effects. For example, a SIRT1 inhibitor and a PPARγ agonist can have additive or synergistic effects on gene expression of one or more PPARγ responsive genes, for example, FGF21 or adiponectin. The combination of agents described herein can have additive or synergistic effects on a disorder, e.g., a disorder described herein. Preferably, the effects are synergistic (e.g., the two agents produce an effect greater than the sum of their individual effects).

A combination of agents described herein can increase the expression of a gene by at least about 10%, at least about 20%, at least about 30%, at least about 40%, at least about 50%, at least about 60%, at least about 70%, at least about 80%, at least about 90%, at least about 95%, at least about 99%, or at least about 100% as compared to the level of expression of the gene under identical conditions but in the absence of the combination. For example, FGF21 or adiponectin expression (e.g., increased expression) can be induced.

When the compositions of this disclosure involve a combination of a SIRT1 inhibitor and a PPARγ agonist, both the SIRT1 inhibitor and the PPARγ agonist should be present at dosage levels of between about 10 to 100%, e.g., between about 10 to 95% of the dosage normally administered in a monotherapy regimen.

Combination therapy can be advantageous, e.g, because the erapeutic effect achieved with the combination can he greater effect achieved by either agent alone, for example, the maximum dose of a first agent may be limited due to toxicity. Thus, the therapeutic effect achieved of that first agent is likewise. The same could be true for a second agent when administered alone. However, if the first agent is administered in combination with the second agent (both, e.g., at their maximum doses), and the two agents have an additive or synergistic effect, the total therapeutic effect achieved by the combination will be greater than that achieved with either agent alone. Similarly, if two agents have additive or synergistic effects when administered in combination, then, to achieve a given therapeutic effect (e.g., an effect that can be achieved by one of the agents when used alone), the doses required of one or both agent when used in combination can be less than the dose required if either of the agents was used alone. This decreased dose of one of both agent could, for example, result in decreased side effects or toxicity caused by one or both of the agents because less is administered.

Upon improvement of a patient's condition, a maintenance dose of a compound, composition or combination of this disclosure may be administered, if necessary. Subsequently, the dosage or frequency of administration, or both, may be reduced, e.g., to about ½ or ¼ or less of the dosage or frequency of administration, as a function of the symptoms, to a level at which the improved condition is retained when the symptoms have been alleviated to the desired level, treatment should cease. Subjects may, however, require intermittent treatment on a long-term basis upon any recurrence of disease symptoms.

It should also be understood that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of active ingredients will also depend upon the particular described compound and the presence or absence and the nature of the additional agent in the composition.

Combinations of a SIRT1 Inhibitor and Second Agent

A SIRT1 inhibitor can be used with a PPARγ agonist, e.g., a PPARγ agonist described herein. The disclosure also includes a composition that contains a SIRT1 inhibitor, e.g., a SIRT1 inhibitor described herein, and a PPARγ agonist e.g., PPARγ agonist described herein.

Further, the disclosure features the use of a SIRT1 inhibitor, e.g., a SIRT1 inhibitor described herein, in combination with a second agent, e.g., for the treatment of a disorder. The disclosure also provides a composition contains a SIRT1 inhibitor in combination with a second agent.

In some aspects, a SIRT1 inhibitor can be used in combination with one of the following agents as a second agent: sitagliptin (e.g., to treat diabetes); metformin (e.g., to treat diabetes); an angiotensin II receptor blocker (e.g., losartan) (e.g., to treat inflammation); retinoid (e.g., to treat diabetes or multiple sclerosis); simvastatin (e.g., to treat atherosclerosis); a statin (e.g., to treat diabetes); a sulfonylurea (e.g., to treat diabetes); natural and synthetic RXR ligands (e.g., all-trans-retinoic acid, 9-cis-retinoic acid, phytanic acid, fenretinide, tazarotene and other derivatives of retinoic acid) (e.g., to treat cancer); dipeptidyl peptidase IV (DPP IV) inhibitor (e.g., LAF-237 (vildagliptin)) (e.g., to treat diabetes); insulin (e.g., to treat diabetes). Further, a SIRT1 inhibitor can be used in combination with a Retinoid X Receptor (RXR) agonist (e.g., 2-[1-3,5,5,8,8-pentamethyl-5,6,7,8-tetrahydro-2-naphthyl)-cyclopropyl]-pyridine-5-carboxylic acid or 4-[1,5,6,7,8-tetrahydro-3,5,5,8,8-pentamethyl-2-naphthalenyl)ethenyl]benzoic acid), e.g., to treat diabetes, hypertriglyceridemia, cardiovascular disease, low levels of lipoprotein lipase, low levels of HDL cholesterol, or to increase adipocyte differentiation (see also EP1426048 for additional examples of RXR agonists). A PPARγ agonist can also be used in combination with the SIRT1 inhibitor and the second agent.

In other aspects, a composition can contain a SIRT1 inhibitor, e.g., a SIRT1 inhibitor described herein, and a second agent, e.g., sitagliptin, metformin, an angiotensin II receptor blocker, retinoid, simvastatin, a statin, a sulfonylurea, a natural or synthetic RXR ligand, a dipeptidyl peptidase IV (DPP IV) inhibitor, insulin, or a Retinoid X Receptor (RXR) agonist. A PPARγ agonist can also be in the composition with the SIRT1 inhibitor and the second agent.

SIRT1 Inhibitors

Non-limiting examples of negative regulators of SIRT1 include: pharmacologic inhibitors (e.g., small molecule inhibitors), dominant negatives (e.g., catalytically inactive forms of Sirt1), and small interfering RNA (siRNA).

SIRT1 inhibitors that can be used in practicing the invention have a general formula (I) and contain a substituted five or six membered ring core containing one or two, respectively, oxygen, nitrogen, or sulfur atoms as a constituent atom of the ring, e.g., X and Y in formula (I) below.

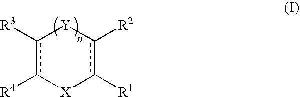

Any ring carbon atom can be substituted. For example, R¹, R², R³, and R⁴ may include without limitation substituted or unsubstituted alkyl, cycloalkyl, alkenyl, alkynyl, heterocyclyl, heterocycloalkenyl, cycloalkenyl, aryl, heteroaryl, etc. The five or six membered ring core may be saturated, i.e. containing no double bonds, or partially or fully saturated, i.e. one or two double bonds respectively. When n=0, “X” may be oxygen, sulfur, or nitrogen, e.g., NR⁷. The substituent R⁷ can be without limitation hydrogen, alkyl, e.g., C1, C2, C3, C4 alkyl, SO₂(aryl), acyl, or the ring nitrogen may form part of a carbamate, or urea group. When n=1, X can be NR⁷, O, or S; and Y can be NR^{7′}, O or S. X and Y can be any combination of heteroatoms, e,g,. N,N, N,O, N, S, etc.

A preferred subset of compounds of formula (I) includes those having one, or preferably, two rings that are fused to the five or six membered ring core, e.g., R¹ and R², together with the carbons to which they are attached, and/or R³ and R⁴, together with the carbons to which they are attached, can form, e.g., C₅-C₁₀ cycloalkyl (e.g., C5, C6, or C7), C₅-C₁₀ heterocyclyl (e.g., C5, C6, or C7), C₅-C₁₀ cycloalkenyl (e.g., C5, C6, or C7), C₅-C₁₀ heterocycloalkenyl (e.g., C5, C6, or C7), C₆-C₁₀ aryl (e.g., C6, C8 or C10), or C₆-C₁₀ heteroaryl (e.g., C5 or C6). Fused ring, combinations may include without limitation one or more of the following:

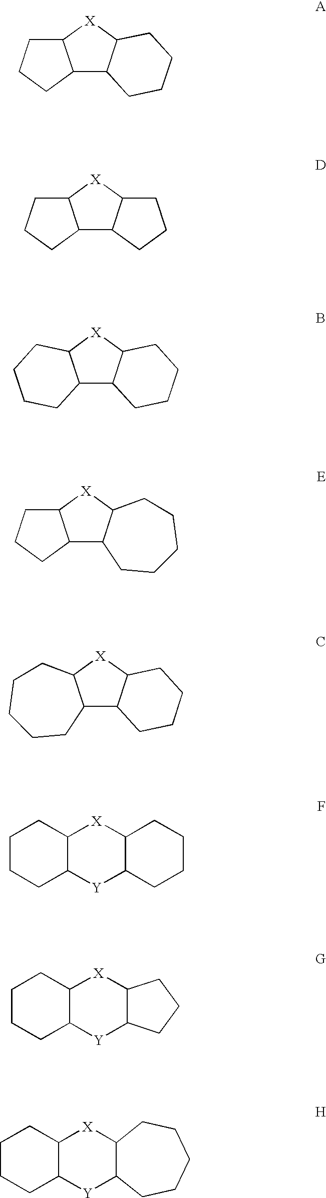

Preferred combinations include B, e.g. having C₆ aryl and C₆ cycloalkenyl (B1), C, e.g. having C₆ aryl and C₇ cycloalkenyl (C1):

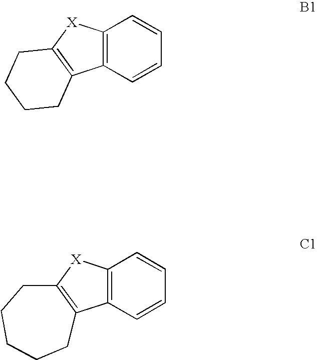

Each of these fused ring systems may be optionally substituted with substituents, which may include without limitation halo, hydroxy, C₁-C₁₀ alkyl (C1,C2,C3,C4,C5,C6,C7,C8,C9,C10) , C₁-C₆ haloalkyl (C1,C2,C3,C4,C5,C6,), C₁-C₁₀ alkoxy (C1,C2,C3,C4,C5,C6,C7,C8,C9,C10), C₁-C₆ haloalkoxy (C1,C2,C3,C4,C5,C6,), C₆-C₁₀ aryl (C6,C7,C8,C9,C10), C₅-C₁₀ heteroaryl (C5,C6,C7,C8,C9,C10), C₇-C₁₂ aralkyl (C7,C8,C9,C10,C11,C12), heteroaralkyl (C7,C8,C9,C10,C11,C12), C₃-C₈ heterocyclyl (C3,C4,C5,C6,C7,C8), C₂-C₁₂ alkenyl (C2,C3,C4,C5,C6,C7,C8,C9,C10,C11,C12), C₂-C₁₂ alkynyl (C2,C3,C4,C5,C6,C7,C8,C9,C10,C11,C12), C₅-C₁₀ cycloalkenyl (C5,C6,C7,C8,C9,C10), C₅-C₁₀ heterocycloalkenyl (C5,C6,C7,C8,C9,C10), carboxy, carboxylate, cyano, nitro, amino, C₁-C₆ alkyl amino (C1,C2,C3,C4,C5,C6,), C₁-C₆ dialkyl amino (C1,C2,C3,C4,C5,C6,), mercapto, SO₃H, sulfate, S(O)NH₂, S(O)₂NH₂, phosphate, C₁-C₄ alkylenedioxy (C1,C2,C3,C4), oxo, acyl, aminocarbonyl, C₁-C₆ alkyl aminocarbonyl (C1,C2,C3,C4,C5,C6,), C₁-C₆ dialkyl aminocarbonyl (C1,C2,C3,C4,C5,C6,), C₁-C₁₀ alkoxycarbonyl (C1,C2,C3,C4,C5,C6,C7,C8,C9,C10), C₁-C₁₀ thioalkoxycarbonyl (C1,C2,C3,C4,C5,C6,C7,C8,C9,C10), hydrazinocarbonyl, C₁-C₆ alkyl hydrazinocarbonyl (C1,C2,C3,C4,C5,C6,), C₁-C₆ dialkyl hydrazinocarbonyl (C1,C2,C3,C4,C5,C6,), hydroxyaminocarbonyl, etc. Preferred substituents include halo (e.g., fluoro, chloro, bromo), C₁-C₁₀ alkyl (e.g., C1, C2, C3, C4, C5, C6, C7, C8, C9, C10), C₁-C₆ haloalkyl (e.g., C1, C2, C3, C4, C5, C6, e.g., CF₃), C₁-C₆ haloalkoxyl (e.g., C1, C2, C3, C4, C5, C6, e,g., OCF₃), or aminocarbonyl. The substitution pattern on the two fused s may be selected as desired, e.g., one ring may be substituted and the other is not, or both rings may be substituted with 1-5 substituents (1,2,3,4,5 substitutents). The number of substituents on each ring may be the same or different. Preferred substitution patterns are shown below:

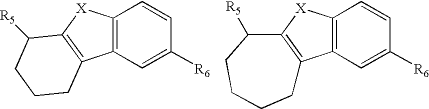

In certain embodiments, when n is 0 and X is NR⁷, the nitrogen substituent R⁷ can form a cyclic structure with one of the fused rings containing, e.g., 4-6 carbons, 1-3 nitrogens, 0-2 oxygens and 0-2 sulfurs. This cyclic structure may optionally be substituted with oxo or C₁-C₆ alkyl.

Combinations of substituents and variables envisioned by this invention are only those that result in the formation of stable compounds. The term “stable,” as used herein, refers to compounds which possess stability sufficient to allow manufacture and which maintains the integrity of the compound for a sufficient period of time to be useful for the purposes detailed herein (e.g., therapeutic or prophylactic administration to a subject).

Exemplary SIRT1 inhibitors include those depicted in Table 1 below*:

TABLE 1
Exemplary SIRT1 inhibitors

| Compound | | Ave. SIRT1 p53-382 |
|---|---|---|
| number | Chemical name | IC50 (μM) |
| 1 | 7-Chloro-1,2,3,4-tetrahydro-cyclopenta[b]indole-3-carboxylic | A |
| | acid amide | |
| 2 | 2,3,4,9-Tetrahydro-1H-b-carboline-3-carboxylic acid amide | C |
| 3 | 6-Bromo-2,3,4,9-tetrahydro-1H-carbazole-2-carboxylic acid | B |
| | amide | |
| 4 | 6-Methyl-2,3,4,9-tetrahydro-1H-carbazole-1-carboxylic acid | A |
| | amide | |
| 5 | 2,3,4,9-Tetrahydro-1H-carbazole-1-carboxylic acid amide | B |
| 6 | 2-Chloro-5,6,7,8,9,10-hexahydro-cyclohepta[b]indole-6- | A |
| | carboxylic acid amide | |
| 7 | 6-Chloro-2,3,4,9-tetrahydro-1H-carbazole-1-carboxylic acid | C |
| | hydroxyamide | |
| 8 | 6-Chloro-2,3,4,9-tetrahydro-1H-carbazole-1-carboxylic acid | A |
| | amide | |
| 9 | 6-Chloro-2,3,4,9-tetrahydro-1H-carbazole-2-carboxylic acid | C |
| | amide | |
| 10 | 1,2,3,4-Tetrahydro-cyclopenta[b]indole-3-carboxylic acid | B |
| | amide | |
| 11 | 6-Chloro-2,3,4,9-tetrahydro-1H-carbazole-1-carboxylic acid (5- | B |
| | chloro-pyridin-2-yl)-amide | |
| 12 | 1,6-Dimethyl-2,3,4,9-tetrahydro-1H-carbazole-1-carboxylic | C |
| | acid amide | |
| 13 | 6-Trifluoromethoxy-2,3,4,9-tetrahydro-1H-carbazole-2- | C |
| | carboxylic acid amide | |
| 14 | 6-Chloro-2,3,4,9-tetrahydro-1H-carbazole-1-carboxylic acid | D |
| | diethylamide | |
| 15 | 6-Chloro-2,3,4,9-tetrahydro-1H-carbazole-1-carboxylic acid | D |
| | carbamoylmethyl-amide | |
| 16 | 8-Carbamoyl-6,7,8,9-tetrahydro-5H-carbazole-1-carboxylic | D |
| | acid | |
| 17 | 6-Methyl-2,3,4,9-tetrahydro-1H-carbazole-1-carboxylic acid | D |
| 18 | 8-Carbamoyl-2,3,4,9-tetrahydro-1H-carbazole-1-carboxylic | D |
| | acid ethyl ester | |
| 19 | [(6-Chloro-2,3,4,9-tetrahydro-1H-carbazole-1-carbonyl)- | D |
| | amino]-acetic acid ethyl ester | |
| 20 | 9-Benzyl-2,3,4,9-tetrahydro-1H-carbazole-1-carboxylic acid | D |
| | amide | |
| 21 | 6-Chloro-2,3,4,9-tetrahydro-1H-carbazole-1-carboxylic acid | D |
| | methyl ester | |
| 22 | 6-Chloro-2,3,4,9-tetrahydro-1H-carbazole-1-carboxylic acid | D |
| 23 | C-(6-Methyl-2,3,4,9-tetrahydro-1H-carbazol-1-yl)-methylamine | D |
| 24 | 6,9-Dimethyl-2,3,4,9-tetrahydro-1H-carbazole-1-carboxylic | D |
| | acid amide | |
| 25 | 7-Methyl-1,2,3,4-tetrahydro-cyclopenta[b]indole-3-carboxylic | D |
| | acid amide | |
| 26 | 6-Chloro-2,3,4,9-tetrahydro-1H-carbazole-1-carboxylic acid | D |
| | ethylamide | |
| 27 | 2-(1-Benzyl-3-methylsulfanyl-1H-indol-2-yl)-N-p-tolyl- | D |
| | acetamide | |
| 28 | N-Benzyl-2-(1-methyl-3-phenylsulfanyl-1H-indol-2-yl)- | D |
| | acetamide | |
| 29 | N-(4-Chloro-phenyl)-2-(1-methyl-3-phenylsulfanyl-1H-indol-2- | D |
| | yl)-acetamide | |
| 30 | N-(3-Hydroxy-propyl)-2-(1-methyl-3-phenylsulfanyl-1H-indol-2- | D |
| | yl)-acetamide | |
| 31 | 2-(1-Benzyl-3-phenylsulfanyl-1H-indol-2-yl)-N-(3-hydroxy- | D |
| | propyl)-acetamide | |
| 32 | 2-(1-Benzyl-3-methylsulfanyl-1H-indol-2-yl)-N-(4-methoxy- | D |
| | phenyl)-acetamide | |
| 33 | 2-(1-Benzyl-1H-indol-2-yl)-N-(4-methoxy-phenyl)-acetamide | D |
| 34 | 2-(1-Methyl-3-methylsulfanyl-1H-indol-2-yl)-N-p-tolyl- | D |
| | acetamide | |
| 35 | 2-(1-Benzyl-3-methylsulfanyl-1H-indol-2-yl)-N-(2-chloro- | D |
| | phenyl)-acetamide | |
| 36 | 2-(1,5-Dimethyl-3-methylsulfanyl-1H-indol-2-yl)-N-(2-hydroxy- | D |
| | ethyl)-acetamide | |
| 37 | (6-Chloro-2,3,4,9-tetrahydro-1H-carbazol-1-yl)-[4-(furan-2- | D |
| | carbonyl)-piperazin-1-yl]-methanone | |
| 38 | 2-(1-Benzyl-1H-indol-2-yl)-N-(2-chloro-phenyl)-acetamide | D |
| 39 | 6-Chloro-2,3,4,9-tetrahydro-1H-carbazole-1-carboxylic acid | D |
| | ethyl ester | |
| 40 | 6-Chloro-9-methyl-2,3,4,9-tetrahydro-1H-carbazole-4- | D |
| | carboxylic acid ethyl ester | |
| 41 | 5,7-Dichloro-2,3,4,9-tetrahydro-1H-carbazole-1-carboxylic | D |
| | acid ethyl ester | |
| 42 | 7-Chloro-2,3,4,9-tetrahydro-1H-carbazole-1-carboxylic acid | D |
| | ethyl ester | |
| 43 | 5,7-Dichloro-2,3,4,9-tetrahydro-1H-carbazole-1-carboxylic | D |
| | acid | |
| 44 | 6-Chloro-9-methyl-2,3,4,9-tetrahydro-1H-carbazole-4- | D |
| | carboxylic acid | |
| 45 | 6-Chloro-9-methyl-2,3,4,9-tetrahydro-1H-carbazole-4- | D |
| | carboxylic acid amide | |
| 46 | 6-Morpholin-4-yl-2,3,4,9-tetrahydro-1H-carbazole-1-carboxylic | D |
| | acid ethyl ester | |
| 47 | 6-Morpholin-4-yl-2,3,4,9-tetrahydro-1H-carbazole-1-carboxylic | D |
| | acid amide | |
| 48 | 6-Bromo-2,3,4,9-tetrahydro-1H-carbazole-1-carboxylic acid | D |
| | ethyl ester | |
| 49 | 6-Fluoro-2,3,4,9-tetrahydro-1H-carbazole-1-carboxylic acid | D |
| | ethyl ester | |
| 50 | 3-Carbamoyl-1,3,4,9-tetrahydro-b-carboline-2-carboxylic acid | D |
| | tert-butyl ester | |
| 51 | 6-Chloro-2,3,4,9-tetrahydro-1H-carbazole-1-carboxylic acid (1- | D |
| | phenyl-ethyl)-amide | |
| 52 | 7,8-Difluoro-2,3,4,9-tetrahydro-1H-carbazole-1-carboxylic acid | D |
| | amide | |
| 53 | 6-bromo-2,3,4,9-tetrahydro-1H-carbazole-1-carboxylic acid | D |
| 54 | 6-hydroxy-2,3,4,9-tetrahydro-1H-carbazole-1-carboxylic acid | C |
| 55 | 6-bromo-2,3,4,9-tetrahydro-1H-carbazole-2-carboxamide | B |
| 56 | 6-chloro-2,3,4,9-tetrahydro-1H-pyrido[3,4-b]indole-1- | C |
| | carboxamide | |
| 57 | 6-bromo-2,3,4,9-tetrahydro-1H-pyrido[3,4-b]indole-1- | D |
| | carboxamide | |
| 58 | 2-acetyl-6-chloro-2,3,4,9-tetrahydro-1H-pyrido[3,4-b]indole-1- | C |
| | carboxamide | |

* Compounds having activity designated with an A have an IC₅₀ of less than 1.0 μM, Compounds having activity designated with a B have an IC₅₀ between 1.0 μM and 10.0 μM. Compounds having activity designated with a C have an IC₅₀ greater than 10.0 μM. Compounds designated with a D were not tested in this assay.

Compounds that can he useful in practicing this invention can be identified through both in vitro (cell and non-cell based) and in vivo methods.

Exemplary compounds are also described, e.g., in US Published Application No. 20060074124. Other exemplary compounds include those described above, for example, Exemplary SIRT1 inhibitors include nicotinamide (NAM), suranim; NF023 (a G-protein antagonist); NF279 (a purinergic receptor antagonist); Trolox (6-hydroxy-2,5,7,8,tetramethylchroman-2-carboxylic acid); (−)-epigallocatechin (hydroxy on sites 3,5,7,3′,4′,5′); (−)-epigallocatechin (hydroxy on sites 3,5,7,3′,4′,5′); (−)-epigallocatechin gallate (Hydroxy sites 5,7,3′,4′,5′ and gallate ester on 3); cyanidin choloride (3,5,7,3′,4′-pentahydroxyflavylium chloride); delphinidin chloride (3,5,7,3′,4′,5′-hexahydroxyflavylium chloride); myricetin (cannabiscetin; 3,5,7,3′,4′,5′-hexahydroxyflavone); 3,7,3′,4′,5′-pentahydroxyflavone; and gossypetin (3,5,7,8,3′,4′-hexahydroxyflavone), all of which are further described in Howitz et al. (2003) Nature 425:191. Other inhibitors, such as sirtinol and splitomicin, are described in Grozinger et al. (2001) Biol. Chem. 276:38837, Dedaiov et al. (2001) PNAS 98:15113 and Hirao et al. (2003) J. Biol. Chem 278:52773. Analogs and derivatives of these compounds can also be used. Additional examples include the natural products guttiferone G (1) and hyperforin (2) as well as the synthetic aristoforin (3), tetrahydrocarbazole compounds, for example, as described in Nayagam et al., (SIRT1 modulating compounds from high-throughput screening as anti-inflammatory and insulin-sensitizing agents, J. Biomol. Screen, 2006, 11, 959-967), those compounds disclosed in US Published Application Nos. 2006-0111435 2007-0043050, 2007-0043050, 2007-0037827, 2007-0037865, 2006-0276393, and 2006-0229265 all of which are incorporated by reference in their entireties herein, are suitable for use in the invention.

SIRT1 Activators:

Activators of SIRT (e.g., SIRT1) are known and can be found, for example in the following US Patent Application Nos. 2007-0014833, 2007-0037809, 2007-0037810, 2007-0037827, 2007-0037865, and 2007-0043050 each of which is incorporated herein by reference.

Synthesis of Compounds

The compounds described herein can be obtained from commercial sources (e.g., Asinex, Moscow, Russia; Bionet, Camelford, England; ChemDiv, SanDiego, Calif.; Comgenex, Budapest, Hungary; Enamine, Kiev, Ukraine; IF Lab, Ukraine; Interbioscreen, Moscow, Russia; Maybridge, Tintagel, UK; Specs, The Netherlands; Timtec, Newark, DE; Vitas-M Lab, Moscow, Russia) or synthesized by conventional methods as shown below using commercially available starting materials and reagents. For example, exemplary compound 4 can be synthesized as shown in Scheme 1 below.

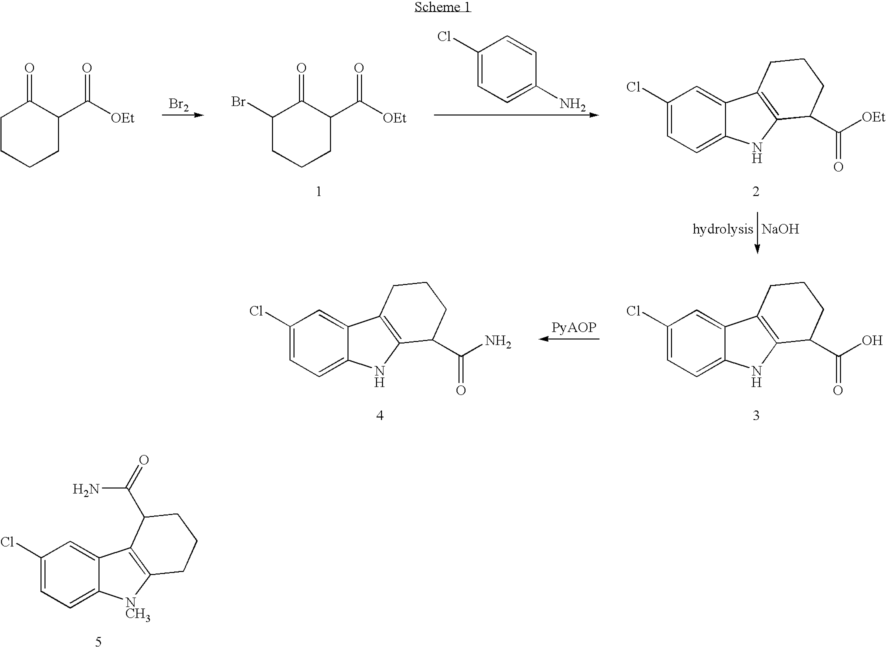

Brominated β-keto ester 1 can be condensed with 4-chloroaniline followed by cyclization can afford indole 2. Ester saponification can afford acid 3. Finally amination with PyAOP can yield the amide 4. Other methods are known in the art, see, e.g., U.S. Pat. No. 3,859,304, U.S. Pat. No. 3,769,298, J. Am. Chem. Soc. 1974, 74, 5495. The synthesis above can be extended to other anilines, e.g., 3,5-dichloroaniline, 3-chloroaniline, and 4-bromoaniline. Regioisomeric products, e.g., 5, may be obtained using N-substituted anilines, e.g., 4-chloro-N-methylaniline.

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies protection and deprotection) useful in synthesizing the compounds described, herein are known in the art and include, for example, those such as described in R. Larock, Comprehensive Organic Transformations, VCH Publishers (1989); T. W. Greene and P. G. K. Wuts; Protective Groups in Organic Synthesis, 2d. Ed., John Wiley and Sons (1991); Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

Techniques useful for the separation of isomers, e.g., stereoisomers are within skill of the art and are described in Eliel, E. L.; Wilen, S. H.; Mander, L. N. Stereochemistry of Organic Compounds, Wiley Interscience, NY, 1994. For example compound 3 or 4 can be resolved to a high enantiomeric excess (e.g., 60%, 70%, 80%, 85%, 90%, 95%, 99% or greater) via formation of diasteromeric salts, e.g. with a chiral base, e.g., (+) or (−) α-methylbenzylamine, or via high performance liquid chromatography using a chiral column. In some embodiments, the crude product 4, is purified directly on a chiral column to provide enantiomerically enriched compound.

For purposes of illustration, enantiomers of compound 4 are shown below.

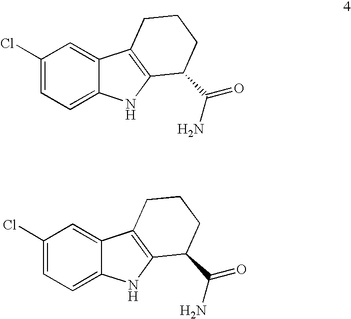

In some instances, the compounds disclosed herein administered where one isomer (e,g the R isomer or S isomer) is present in high enantiomeric excess. In general, the isomer of compound 4 having a negative optical rotation, e.g., −14.1 (c=0.33, DCM) or [α]ₙ²⁵ −41.18° (c 0.960, CH₃OH) has greater activity against the SIRT1 enzyme than the enantiomer that has a positive optical rotation of +32.8 (c=0.38, DCM) or [α]_{D}²⁵ +22.72° (c 0.910, CH₃OH). Accordingly, in some instances, it is beneficial to administer to a subject a compound 4 having a high enantiomeric excess of the isomer having a negative optical rotation to treat a disease.

While the enantiomers of compound 4 provide one example of a stereoisomer, other stereoisomers are also envisioned, for example as depicted in compounds 6 and 7 below.

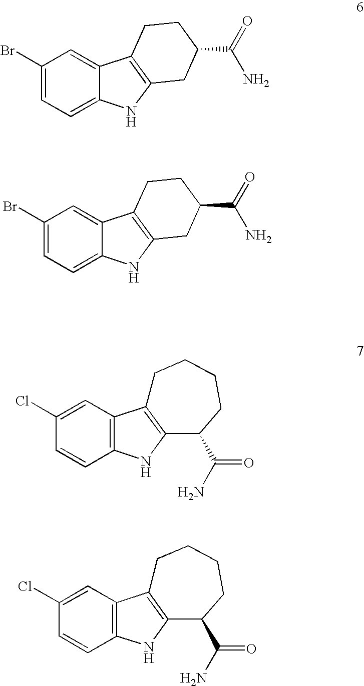

As with the compound of formula 4, in some instances it is beneficial to administer to a subject an isomer of compounds 6 or 7 that has a greater affinity for SIRT1 than its enantiomer. For example, in some instances, it is beneficial to administer a compound 7, enriched with the (−) optical rotamer, wherein the amide (or other substituent) has the same configuration as the negative isomer of compound 4.

In some instances, it is beneficial to administer a compound having one of the following structures where the stereochemical structure of the amide (or other substituent) corresponds to the amide in compound 4 having a negative optical rotation.

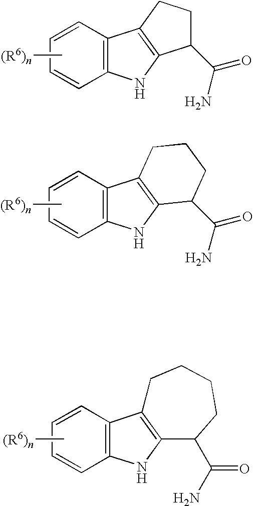

(n is an integer from 0 to 4.)

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as “split and pool” or “parallel” synthesis techniques solid-phase, and solution-phase techniques, and encoding techniques (see, for example, Czarnik, A. W., Curr. Opin. Chem. Bio., (1997) 1, 60. Thus, embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Other examples of SIRT1 inhibitors that can be used in the compositions and methods described herein include those disclosed in US Patent Application No. 2005-0250794, the contents of which are hereby incorporated by reference in its entirety.

PPARγ

The peroxisome proliferator-activated receptors (PPARs) are a group of nuclear receptor proteins that function as transcription factors regulating the expression of genes. PPARs play essential roles in the regulation of cellular differentiation, development, and metabolism (carbohydrate, lipid, and protein) of higher organisms.

Three types of PPARs have been identified: alpha, gamma, and delta (beta):

α (alpha)—expressed in kidney, heart, muscle, adipose tissue, and others

β/δ (beta/delta)—expressed in many tissues but markedly in brain, adipose tissue, and skin

γ (gamma)—although transcribed by the same gene, this PPAR through alternative splicing is expressed in three forms:

γ1—expressed in virtually all tissues, including heart, muscle, colon, kidney, pancreas, and spleen

γ2—expressed mainly in adipose tissue (30 amino acids longer)

γ3—expressed in macrophages, large intestine, white adipose tissue.

PPARs heterodimerize with the retinoid X receptor (RXR) and bind to specific regions on the DNA of target genes. These DNA sequences are termed PPREs (peroxisome proliferator hormone response elements). The DNA consensus sequence is AGGTCAXAGGTCA, with X being a random nucleotide.

Peroxisome proliferator-activated receptor gamma (PPAR-gamma; PPARgamma; PPARg; PPARγ), also known as NR1C3 (nuclear receptor subfamily 1, group C, member 3) is a nuclear receptor encoded by the PPARG gene.

The protein encoded by the PPARG gene is PPAR-gamma and is a regulator of adipocyte differentiation. Additionally, PPAR-gamma has been implicated in the pathology of numerous diseases including obesity, diabetes, atherosclerosis and cancer.

The protein contains 505 amino acids, and has a weight of about 57620 Da.

PPARγ can form a heterodimer with the retinoic acid receptor RXRA called adipocyte-specific transcription factor ARF6. PPARγ can interact with NCOA6 coactivator, leading to a strong increase in transcription of target genes. PPARγ can interact with coactivator PPARBP, leading to a mild increase in transcription of target genes. PPARγ can interact with NOCA7 in a ligand-inducible manner. PPARγ can interact with NCOA1 LXXLL motifs and with TGFB1I1 PPARγ is found in the nucleus.

PPARγ encoding sequences include:

| | |
|---|---|
| U79012; AAC51248.1; —; mRNA. | [EMBL/GenBank/DDBJ] |
| | [CoDingSequence] |
| U63415; AAB04028.1; —; mRNA. | [EMBL/GenBank/DDBJ] |
| | [CoDingSequence] |
| D83233; BAA18949.1; —; mRNA. | [EMBL/GenBank/DDBJ] |
| | [CoDingSequence] |
| L40904; AAA80314.2; ALT_INIT; mRNA. | [EMBL/GenBank/DDBJ] |
| | [CoDingSequence] |
| AB005526; BAA23354.1; ALT_INIT; | [EMBL/GenBank/DDBJ] |
| Genomic_DNA. | [CoDingSequence] |
| X90563; CAA62152.1; ALT_INIT; mRNA, | [EMBL/GenBank/DDBJ] |
| | [CoDingSequence] |
| X90563; CAA62153.1; —; mRNA, | [EMBL/GenBank/DDBJ] |
| | [CoDingSequence] |
| AY157024; AAN38992.2; —; | [EMBL/GenBank/DDBJ] |
| Genomic_DNA. | [CoDingSequence] |
| BT007281; AAP35945.1; ALT_INIT; mRNA. | [EMBL/GenBank/DDBJ] |
| | [CoDingSequence] |
| BC006811; AAH06811.1; ALT_INIT; mRNA. | EMBL/GenBank/DDBJ] |
| | [CoDingSequence] |

An exemplary PPARγ amino acid sequence is

(SEQ ID NO: 1)

```
                10 20 30 40 50 60
MGETLGDSPI DPESDSFTDT LSANISQEMT MVDTEMPFWP TNFGISSVDL SVMEDHSHSF
                70 80 90 100 110 120
DIKPFTTVDF SSISTPHYED IPFTRTDPVV ADYKYDLKLQ EYQSAIKVEP ASPPYYSEKT
              130 140 150 160 170 180
QLYNKPHEEP SNSLMAIECR VCGDKASGFH YGVHACEGCK GFFRRTIRLK LIYDRCDLNC
              190 200 210 220 230 240
RIHKKSRNKC QYCRFQKCLA VGMSHNAIRF GRMPQAEKEK LLAEISSDID QLNPESADLR
              250 260 270 280 290 300
ALAKHLYDSY IKSFPLTKAK ARAILTGKTT DKSPFVIYDM NSLMMGEDKI KFKHITPLQE
              310 320 330 340 350 360
QSKEVAIRIF QGCQFRSVEA VQEITEYAKS IPGFVNLDLN DQVTLLKYGV HEIIYTMLAS
              370 380 390 400 410 420
LMNKDGVLIS EGQGFMTREF LKSLRKPFGD FMEPKFEFAV KFNALELDDS DLAIFAIVII
              430 440 450 460 470 480
LSGDRPGLLN VKPIEDIQDN LLQALELQLK LNHPESSQLF AKLLQKMTDL RQIVTEHVQL
              490 500
LQVIKKTETD MSLHPLLQEI YKDLY
```

PPARγ Agonists

PPARγ agonists include thiazolidinediones (TZDs) and non-TZD compounds. A PPARγ agonist can be used in combination with a SIRT1 inhibitor, e,g., to treat a metabolic disorder, diabetes, a neoplastic disorder such as cancer, artenoscierosis, obesity, dyslipidentia, inflammation, cardiovascular disorders, ischemia, or to promote angiogenesis.

TZDs. Thiazolidinediones (TZDs) act by binding to PPARs (peroxisome) proliferator activated receptors), a group of receptor molecules inside the cell nucleus, specifically PPARγ (gamma). The normal ligands for these receptors are free fatty acids (FFAs) and eicosanoids. When activated, the receptor migrates to the DNA, activating transcription of a number of specific genes.

By activating PPARγ, insulin resistance is decreased, adipocyte differentiation is modified, VEGF-induced angiogenesis is inhibited, leptin levels decrease (leading to an increased appetite), levels of certain interleukins (e.g. IL-6) fall, and adiponectin levels rise.

Chemically, the members of this class are derivatives of the parent compound thiazolidinedione, and include: rosiglitazone (Avandia); pioglitazone (Actos); troglitazone (Rezulin), ciglitazone, englitazone, BM 13.1258, BM 15.2054, and derivatives thereof. Another TZD analog is THR-0921. Additional TZDs are described in U.S. Pat. No. 7,323,481 and include:

(1) 5-{4-[2-(5-Methyl-2-phenyloxazol-4-yl)-ethoxy]-benzo[b]thiophen-7-ylmethyl}-thiazolidine-2,4-dione=BM 13.1258

(2) 5-{4-[2-(5-Methyl-2-(thien-2-yl)oxazol-4-yl)-ethoxy]-benzo[b]thiophen-7-ylmethyl}-thiazolidine-2,4-dione=BM 15.2054

(3) 5-{4-[2-(5-Methyl-2-phenyloxazol-4-yl)-ethoxy]-naphth-1-ylmethyl}-thiazol-idine-2,4-dione

(4) 5-{4-[2-(2-(4-Fluorophenyl)-5-methyloxazol-4-yl)-ethoxyl-benzo[b]thiophen-7-ylmethyl}-thiazolidine-2,4-dione

(5) 5-{4-[2-(2-(4-Chlorophenyl)-5-methyloxazol-4-yl)-ethoxyl-benzo[b]thiophen-7-ylmethyl}-thiazolidine-2,4-dione

(6) 5-{4-[2-(5-Methyl-2-(4-trifluoromethylphenyl)-oxazol-4-yl)-ethoxyl-benzo[-b]thiophen-7-ylmethyl}-thiazolidine-2,4-dione

(7) 5-{4-[2-(2-(4-Hydroxyphenyl)-5-methyloxazol-4-yl)-ethoxy]-benzo[b]thiophe-n-7-ylmethyl}-thiazolidine-2,4-dione

(8) 5-{4-[2-(5-Methyl-2-(thien-2-yl)-oxazol-4-yl)-ethoxy]-naphthalen-1-ylmethyl-}-thiazolidine-2,4-dione.

Non-TZDs. PPARγ is also mildly activated by certain NSAIDs (such as ibuprofen) and indoles.

“Dual,” “balanced” or “pan” PPAR ligands, which bind two or more PPAR isoforms, include the compounds aleglitazar, muraglitazar and tesaglitazar.

PPARγ can be activated by PGJ₂ (a prostaglandin) (15-deoxy-Δ_{12,14}-prostaglandin J₂(15d-PGJ2)).

Other non-TZD agonists of PPARγ include: GW1929, GW7845, RWJ-348260, AK109, mono-2-ethyhexyl phthalate, GI262570, eicosanoids, tetrahydroisoquinoline PPARγ agonists, and heterocyclic compounds (as described, e.g., in U.S. Pat. No. 6,462,046). See also, the compound of WO 2007/058504. The structure of GW1929 is:

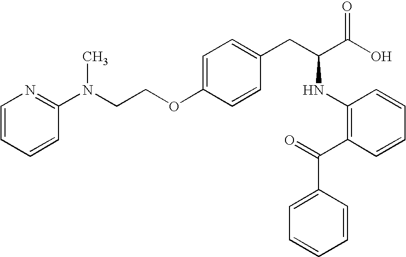

PPARγ Antagonists

PPARγ antagonists can be used, e.g., in combination with a SIRT1 activator, to treat cancer and/or inhibit angiogenesis.

PPARg antagonists include:

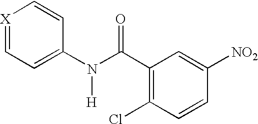

where X can be a CH or N. In one embodiment, X may be a CH, in another embodiment, X maybe a N. Other examples include genistein, T0070907, hisphenol A diglycidyl ether (BADGE), GW-9662, PD 068235, SR-202, LG 100641, lysophosphatidic acid (LPA), tea catechins, extracts from Hibiscus, oleic acid, 10-nonadecenoic acid, 11-eicosenoic acid, heneicosanoic acid, Red Yeast Rice, and tannic acid, and combinations thereof. See also WO 2006/099479 and WO 2007/070523.

Metabolic Disorders

A SIRT1 inhibitor, e.g., a SIRT1 inhibitor described herein, can be used to treat metabolic disorders, such as diabetes, obesity, and metabolic syndrome. A PPARγ agonist, e.g., a PPARγ agonist described herein, can be used in combination with the SIRT1 inhibitor.

A “metabolic disorder” is a disease or disorder characterized by an abnormality or malfunction of metabolism. One category of metabolic disorders is disorders of glucose or insulin metabolism.

Diabetes

The invention includes methods of treating and preventing diabetes. Examples of diabetes include insulin dependent diabetes mellitus and non-insulin dependent diabetes. For example, the method includes administering to a subject having diabetes or at risk of diabetes a SIRT1 inhibitor in combination with a PPARγ agonist, e.g., as described herein. In some instances, a subject can be identified as being at risk of developing diabetes by having impaired glucose tolerance (IGT), or fasting hyperglycemia.

For example, a SIRT1 inhibitor in combination with a PPARγ agonist, e.g., as described herein, can be administered to a subject in a therapeutically effective amount to decrease gluconeogenesis, improve glycemic control (i.e., lower fasting blood glucose), or normalize insulin sensitivity. The SIRT1 inhibitor and PPARγ agonist can be administered to a subject that has or is at risk for diabetes.

Insulin dependent diabetes mellitus (type 1 diabetes) is an autoimmune disease, where insulitis leads to the destruction of pancreatic β cells. At the time of clinical onset of type 1 diabetes mellitus, significant number of insulin producing β cells are destroyed and only 15% to 40% are still capable of insulin production (McCulloch et al. (1991) Diabetes 40:673-679). β-cell failure results in a life long dependence on daily insulin injections and exposure to acute and late complications of the disease.

Type 2 diabetes mellitus is a metabolic disease of impaired glucose homeostasis characterized by hyperglycemia, or high blood sugar, as a result of defective insulin action which manifests as insulin resistance, defective insulin secretion, or both. A subject (e.g. patient) with type 2 diabetes mellitus has abnormal carbohydrate, lipid, and protein metabolism associated with insulin resistance and/or impaired insulin secretion. The disease leads to pancreatic beta cell destruction and eventually absolute insulin deficiency. Without insulin, high glucose levels remain in the blood. The long term effects of high blood glucose include blindness, renal failure, and poor blood circulation to these areas, which can lead to leg, foot or ankle amputations. Early detection is critical in preventing patients from reaching this degree of severity. The majority of subjects with diabetes have the non-insulin dependent form of diabetes, currently referred to as type 2 diabetes mellitus.

The invention also includes methods of treating disorders related to or resulting from diabetes, for example end organ damage, diabetic gastroparesis, diabetic neuropathy, cardiac dysrythmia, etc.

Exemplary molecular models of type 2 diabetes include: a transgenic mouse having defective Nkx.-2.2 or Nkx-6.1; (U.S. Pat. No. 6,127,598); Zucker Diabetic Fatty fa/fa (ZDF) rat. (U.S. Pat. No. 6,569,832); and Rhesus monkeys, which spontaneously develop obesity and subsequently frequently progress to overt type 2 diabetes (Hotta et al., Diabetes, 50:1126-33 (2001); and a transgenic mouse with a dominant-negative IGF-I receptor (KR-IGF-IR) having type 2 diabetes-like insulin resistance.

Metabolic Syndrome

The invention provides a method of treating metabolic syndrome, including administering to a subject that has or is at risk for metabolic syndrome a SIRT1 inhibitor in combination with a PPARγ agonist, e.g., as described herein.

The metabolic syndrome (e.g., Syndrome X) is characterized by a group of metabolic risk factors in one person. The risk factors include: central obesity fat tissue in and around the abdomen), atherogenic dyslipidemia (blood fat disorders—mainly high triglycerides and low HDL cholesterol—that foster plaque buildups in artery walls); insulin resistance or glucose intolerance (the body can't properly use insulin or blood sugar); prothrombotic state (e.g., high fibrinogen or plasminogen activator inhibitor [−1] in the blood); raised blood pressure (i.e., hypertension) (130/85 mmHg or higher); and proinflammatory state (e.g., elevated, high-sensitivity C-reactive protein in the blood).

The underlying causes of this syndrome are being overweight/obesity, physical inactivity and genetic factors. People with metabolic syndrome are at increased risk of coronary heart disease, other diseases related to plaque buildups in artery walls (e.g., stroke and peripheral vascular disease) and type 2 diabetes. Metabolic syndrome is closely associated with a generalized metabolic disorder called insulin resistance, in which the body can't use insulin efficiently.

Cancer

The invention includes methods of treating and preventing cancer. For example, the method includes administering to a subject having cancer or at risk of cancer a SIRT1 inhibitor, e.g., a SIRT1 inhibitor described herein, in combination with a PPARγ agonist, e.g., a PPARγ agonist described herein.

The disclosure also provides methods of treating cancer by administering a SIRT1 activator, e.g., a SIRT1 activator described herein, to a subject having cancer or at risk of cancer. For example, the SIRT1 activator inhibits HIF-1 activity and/or decreases expression of proteins involved in angiogenesis. In some aspects, the SIRT1 activator can be administered with a PPARγ antagonist.

The compounds and combinations of the invention can be used in the treatment of cancer. As used herein, the terms “cancer,” “hyperproliferative,” “malignant,” and “neoplastic” are used interchangeably, and refer to those cells in an abnormal state or condition characterized by rapid proliferation or neoplasm or decreased apoptosis. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

The common medical meaning of the term “neoplasia” refers to “new cell growth” that results as a loss of responsiveness to normal growth controls, e.g., to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant, or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using a compound described herein.

The subject method can be useful in treating cancers of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, cast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadeneocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies which may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

The compositions, combinations, and methods described herein can also be used to treat pre-cancerous conditions, such as pre leukemic syndrome myelodysplasia, benign masses of cells, erythroplasia, leukoplakia, lymphomatoid granulomatosis, lymphomatoid papulosis, preleukemia, uterine cervical dysplasia, xeroderma pigmentosum.

Dyslipidemia

The invention includes methods of treating and preventing dyslipidemia. For example, the method includes administering to a subject having dyslipidemia or at risk of dyslipidemia a SIRT1 inhibitor, e.g., a SIRT1 inhibitor described herein. In some aspects, the methods include administering the SIRT1 inhibitor in combination with a PPARg agonist, e.g., a PPARγ agonist described herein.

Dyslipidemia is a disruption of the amount of lipids in the blood of a subject. A common form of dyslipidemia is hyperlipidemia, wherein a subject has an elevation of lipids in the blood, for example, caused by diet, lifestyle, genetics, or a combination thereof. Lipid and lipoprotein abnormalities are common and can be a risk factor for cardiovascular disease due to the influence of cholesterol, one of the most clinically relevant lipid substances, on atherosclerosis. In addition, some forms of hyperlipidemia can predispose a subject to acute pancreatitis. Hyperlipidemias are classified according to the Fredrickson classification which is based on the pattern of lipoproteins on electrophoresis or ultracentrifugation. It was later adopted by the World Health Organization (WHO).

Obesity

The invention includes methods of treating and preventing obesity. For example, the method includes administering to a subject having obesity or at risk of obesity a SIRT1 inhibitor, e.g., a SIRT1 inhibitor described herein, in combination with a PPARγ agonist, e.g., a PPARγ agonist described herein.

“Obesity” refers to a condition in which a subject has a body mass index (weight by height squared) of 30 kg/m² or higher. “Overweight” refers to a condition in subject has a body mass index of greater or equal to 25. As used herein, the body mass index and other definitions are according to the “NIH Clinical Guidelines on the Identification and nation, and Treatment of Overweight and Obesity in Adults” (1998). In particular, obesity can lead to type 2 diabetes in successive phases. Clinically, these phases can be characterized as normal glucose tolerance, impaired glucose tolerance, hyperinsulinemic diabetes, and hypoinsulinemic diabetes. Such a progressive impairment of glucose storage correlates with a rise in basal glycemia.

Exemplary models for the treatment of obesity include two primary animal model systems: 1) diet-induced obesity (DIO) caused by feeding rodents ˜60% fat content of caloric intake. Animals treated for up to 12-16 weeks on this type of diet gain substantial body weight (>50% increase), accumulate excessive fat mass, become hyperglycemic, hyperinsulinemic and insulin resistant. In this model compounds can be tested prior to the initiation of the diet or at any time during development of obesity and 2) db/db mutant mice (leptin receptor spontaneous mutant). These animals exhibit a similar phenotype as the DIO animals only more severe with regard to various readouts. Animals can be treated similar to the DIO model. As a surrogate readout of SIRT11 inhibitor activity, sister animals can be sacrificed at specific intervals in the treatment regimen and assessed biochemically for increased acetylation status of FoxO1 proteins in various tissues, such as liver, muscle and white adipose tissue.

Inflammation

The invention includes methods of treating and preventing inflammation (e.g., acute and/or chronic inflammation). For example, the method includes administering to a subject having inflammation or at risk of inflammation a SIRT1 inhibitor, e.g., a SIRT1 inhibitor described herein. In some aspects, the methods include administering the SIRT1 inhibitor in combination with a PPARγ agonist, e.g., a PPARγ agonist described herein.

Acute inflammation is a short-term process which characterized by the classic signs of inflammation—swelling, redness, pain, heat, and loss of function—due to the infiltration of the tissues by plasma and leukocytes. It occurs as long as the injurious stimulus is present and ceases once the stimulus has been removed, broken down, or walled off by scarring (fibrosis). The process of acute inflammation is initiated by the blood vessels local to the injured tissue, which alter to allow the exudation of plasma proteins and leukocytes into the surrounding tissue. The increased flow of fluid into the tissue causes the characteristic swelling associated with inflammation since the lymphatic system doesn't have the capacity to compensate for it, and the increased blood flow to the area causes the reddened color and increased heat. The blood vessels also alter to permit the extravasation of leukocytes through the endothelium and basement membrane constituting the blood vessel. Once in the tissue, the cells migrate along a chemotactic gradient to reach the site of injury, where they can attempt to remove the stimulus and repair the tissue. Several biochemical cascade systems, consisting of chemicals known as plasma-derived inflammatory mediators, act in parallel to propagate and mature the inflammatory response. These include the complement system, coagulation system and fibrinolysis system.

Finally, down-regulation of the inflammatory response concludes acute inflammation. Removal of the injurious stimulus halts the response of the inflammatory mechanisms, which require constant stimulation to propagate the process. Additionally, many inflammatory mediators have short half lives and are quickly degraded in the tissue, helping to quickly cease the inflammatory response once the stimulus has been removed.

Chronic inflammation is an inflammatory immune response of prolonged duration that eventually leads to tissue damage. Chronic inflammation is differentiated from acute inflammation by extended duration, lasting anywhere from a week to an indefinite time frame. The exact nature of chronic inflammation depends on the causative agent and the body's attempts to ameliorate it.

Chronic inflammation may develop as a progression from acute inflammation if the original stimulus persists or after repeated episodes of acute inflammation. Examples of diseases that can cause chronic inflammation include tuberculosis, chronic cholecystitis, bronchiectasis, rheumatoid arthritis, Hashimoto's thyroiditis, inflammatory bowel disease (ulcerative colitis and Crohn's disease), silicosis and other Pneumoconiosis and an implanted foreign body in a wound among many others.

Arteriosclerosis

The invention includes methods of treating and preventing arteriosclerosis. For example, the method includes administering to a subject having arteriosclerosis or at risk of arteriosclerosis a SIRT1 inhibitor, e.g., a SIRT1 inhibitor described herein. In some aspects, the methods include administering the SIRT1 inhibitor in combination with a PPARγ agonist, e.g., a PPARγ agonist described herein.

Healthy arteries are flexible, strong and elastic. Over time, pressure can make artery walls thick and stiff—sometimes restricting blood flow to organs and tissues.

Arteriosclerosis (hardening (sclerosis) of the arteries (arterio-)) is a general term for several diseases in which the wall of an artery becomes thicker and less elastic. Atherosclerosis is a type of arteriosclerosis.

Although atherosclerosis is often considered a heart problem, it can affect arteries anywhere in the body. For example:

When arteries leading to your limbs are affected, circulation problems in arms and legs, called peripheral arterial disease, may develop.

When arteries to the heart are affected, coronary artery disease, chest pain (angina) or a heart attack may develop.

When arteries supplying blood to the brain are affected, a transient ischemic attack (TIA) or stroke may occur.

Atherosclerosis can also lead to a bulge in the wall of an artery (aneurysm).

Atherosclerosis develops gradually. There are usually no signs or symptoms until an artery is so narrowed or clogged that it can't supply adequate blood to organs and/or tissues. Sometimes a blood clot completely obstructs blood flow.

The specific signs and symptoms depend on which arteries are affected. For example:

Coronary arteries. Obstruction of the arteries to coronary arteries may cause symptoms of heart attack, such as chest pain.

Arteries supplying the brain. Obstruction of the carotid arteries in the neck may cause symptoms of stroke, such as sudden numbness, weakness or dizziness.

Arteries in the arms and legs. Obstruction of the arteries supplying blood to arms and legs may cause symptoms of peripheral arterial disease, such as leg pain walking (intermittent claudication).

Atherosclerosis is a, slow, progressive disease that may begin as early as childhood. It is a chronic inflammatory response in the walls of arteries, in part due to the accumulation of macrophage white blood cells and promoted by low density (especially small particle) lipoproteins (plasma proteins that carry cholesterol and triglycerides) without adequate removal of fats and cholesterol from the macrophages by functional high density lipoproteins. It is commonly referred to as a “hardening” or “furring” of the arteries. Although the exact cause is unknown, atherosclerosis may start with damage or injury to the inner layer of an artery. The damage may be caused by various factors, including: high blood pressure, high cholesterol, an irritant (such as nicotine), and diseases such as diabetes.

Once the inner wall of an artery is damaged, platelets often clump at the injury site to try to repair the artery. Over time, fatty deposits (plaques) made of cholesterol and other cellular waste products also accumulate and harden, narrowing the space in arteries. Organs and tissues that are served by these narrowed vessels don't get an adequate supply of blood.

Hardening of the arteries occurs over time. In addition to simply getting older, factors that increase the risk of atherosclerosis include: high blood pressure, high cholesterol, diabetes, obesity, smoking, and a family history of aneurysm or early heart disease.

Cardiovascular Disorders

The invention includes methods of treating and preventing cardiovascular disease. For example, the method includes administering to a subject having cardiovascular disease or at risk of cardiovascular disease a SIRT1 inhibitor, e.g., a SIRT1 inhibitor described herein. In some aspects, the methods include administering the SIRT1 inhibitor in combination with a PPARγ agonist, e.g., a PPARγ agonist described herein.

Cardiovascular disease refers to the class of diseases that involve the heart or blood vessels (arteries and veins). While the term technically refers to any disease that affects the cardiovascular system, it is usually used to refer to those related to atherosclerosis (arterial disease).

By the time that heart problems are detected, the underlying cause (atherosclerosis) is usually quite advanced, having progressed for decades. There is therefore increased emphasis on preventing atherosclerosis by modifying risk factors, such as healthy eating, exercise and avoidance of smoking.

There are many risk factors which associate with various forms of cardiovascular disease. These include: age, gender (men under the age 64 are much more likely to die of coronary heart disease than women), genetic factors/family history of cardiovascular disease, race (or ethnicity), environment, tobacco smoking, insulin resistance, diabetes mellitus, hypercholesterolemia (elevated cholesterol levels), abnormal lipoprotein particle profile (cholesterol subtypes), obesity, high blood pressure, sleep deprivation, elevated heart rate, physical inactivity/sedentary lifestyle, absence of key nutritional elements, such as omega-3 fatty acids and polyphenol antioxidants, stress, depression, and periodontal disease.

Hypoxia

Ischemia and reperfusion (I/R)-induced tissue injury are major causes of mortality and morbidity. I/R injury can develop, e.g., as a consequence of hypotension, shock, or bypass surgery leading to end-organ failure such as acute renal tubular necrosis, liver failure, and bowel infarct. I/R injury can also develop as a result of complications of vascular disease such as stroke and myocardial infarction. In addition, multiple subclinical I/R incidents can induce cumulative tissue injury leading to chronic degenerative diseases such as vascular dementia, ischemic cardiomyopathy, and renal insufficiency. Hypoxia and oxidative stress associated with I/R are common causes of tissue injury accounting for organ damage in stroke, myocardial infarction, ischemic bowel disease, and kidney and liver failure. One of the major mechanisms by which the cells control gene expression during low oxygen involves the activation of transcription factor hypoxia-inducible factor 1 (HIF1; also referred to as HIF-1 alpha), which is quickly degraded during normoxic conditions. Activation of HIF1 leads to transcription of several target genes such as vascular endothelial growth factor (VEGf; e.g., VEGF-alpha), erythropoietin, nitric oxide synthase, and several antioxidant enzyme systems such as superoxide dismutase, and heme-oxygenase-1 (HO-1), which may provide protection against I/R injury. Hypoxia can induce angiogenesis. Further, HIF1 alpha activity can increase expression of angiogenic factors.

As described herein, inhibition of SIRT1 leads to increased expression of genes that are also induced under hypoxic conditions. A SIRT1 inhibitor, e.g., a SIRT1 inhibitor described herein, can be used to treat conditions involving hypoxia, e.g., ischemia, e.g., to activate HIF1 activity, e.g., and upregulate transcription of VEGF-alpha, e.g., to promote angiogenesis. The SIRT1 inhibitor can be administered to a subject that has or is at risk for hypoxia (e.g., ischemia or n I/R injury). In some aspects, the methods include administering the SIRT1 inhibitor in combination with a PPARγ agonist, e.g., a PPARγ agonist described herein.

Local growth and metastasis of a large variety of malignant tumors depend on neoangiogenesis. The process of tumor angiogenesis is largely based on the production and secretion of angiogenesis factors such as vascular endothelial growth factor (VEGF), fibroblast growth factor (FGF), and interleukin (IL)-8. Tissue hypoxia has been shown to play a key role for the induction of angiogenic factors. Even after neovascularization, tumor areas may remain under low oxygen tension, for example, because of inadequate vascularization after neoangiogenesis. Thus, hypoxic areas remain a constant feature of malignant tumors and metastases. Among angiogenesis factors that have been identified to be inducible by hypoxia are fibroblast growth factor, VEGF, platelet-derived growth factor, IL-8, and angiogenin. Hypoxia-inducible factor (HIF)-1 is a major contributor to gene transcription of hypoxia-inducible genes.

As SIRT1 inhibition leads to increased expression of genes that are also induced under hypoxic conditions, a SIRT1 activator, e.g., a SIRT1 activator described herein, can be used to inhibit the expression of genes induced by hypoxia, e.g., in tumors, e.g., to inhibit or decrease angiogenesis. A SIRT1 activator can be used to treat cancer, e.g., by blocking tumor angiogenesis. A SIRT1 activator can be administered to a subject that has or is at risk for cancer. A PPARγ antagonist can optionally be used with the SIRT1 activator.

Pharmaceutical Compositions

The SIRT1 modulator inhibitor or activator) and, in some embodiments, the other agent, e.g., the PPARγ modulator (e.g., agonist or antagonist) or other agent described herein, may be formulated in separate dosage forms. Alternatively, to decrease the number of dosage forms administered to a subject, each agent may be formulated together in any combination. As one example, the SIRT1 inhibitor may be formulated in one dosage form while the PPARγ agonist is formulated in another dosage form or the PPARγ agonist may be formulated together with the SIRT1 inhibitor. The SIRT1 inhibitor can be dosed, for example, before, after or during the dosage of the PPARγ agonist.

The agent(s) (e.g., the SIRT1 inhibitor and/or PPARγ agonist) can be formulated into a pharmaceutical composition, either separately or together, for example, with one or more pharmaceutically acceptable carriers, adjuvants, or vehicles. Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered enterally (e.g., orally), parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspension, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase and combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches included in this invention.

The pharmaceutical composition of this invention may be by nasal aerosol inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition containing the agents (e.g., the SIRT1 inhibitor and the PPARγ agonist) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). See Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

Also within the invention is a transdermal patch to deliver the combinations described herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., rubbing,) the adhesive or device.

In some cases, e.g., when dominant negative forms of SIRT1 are used to practice the invention, these agents can be administered via gene therapy techniques (e.g., via adenoviral or adeno-associated virus delivery).

When the compositions of this invention comprise a combination of agents (e.g., a SIRT1 inhibitor and a PPARγ agonist), both the compound and the additional agent should be present at dosage levels of between about 1 to 100%, and more preferably between about 5 to 95% of the dosage normally administered in a monotherapy regimen. For example, the PPARγ agonist may be administered separately, as part of a multiple dose regimen, from the SIRT1 inhibitor of this invention. Alternatively, those agents may be part of a single dosage form, mixed together with the compounds of this invention in a single composition.

A subject can be, e.g., a mammal. The term “mammal” includes organisms, which include mice, rats, cows, sheep, pigs, rabbits, goats, horses, monkeys, dogs, cats, and preferably humans.

The term “treating” refers to administering a therapy in an amount, manner, and/or mode effective to improve or prevent a condition, symptom, or parameter associated with a disorder (e.g., a disorder described herein) or to prevent onset, progression, or exacerbation of the disorder, to either a statistically significant degree or to a degree detectable to one skilled in the art. Accordingly, treating can achieve therapeutic and/or prophylactic benefits. An effective amount, manner, or mode can vary depending on the subject and may be tailored to the subject.

A “therapeutically effective amount” or an amount required to achieve a “therapeutic effect” can be determined based on the effect of the administered agent(s). A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition is outweighed by the therapeutically beneficial effects.

The combination described herein can be administered, e.g., once or twice daily, or about one to four times per week, or preferably weekly, biweekly, or monthly, e.g., for between about 1 to 10 weeks (e.g., between 2 to 8 weeks or between about 3 to 7 weeks, or for about 4, 5, or 6 weeks) or for one, two, three, four, five, seven., eight, nine, ten, eleven, twelve, or more months (e.g., for up to 24 months). The skilled artisan will appreciate that certain factors may influence the dosage and timing required to effectively treat a subject, including but not limited to the severity of the disease or disorder, formulation, route of delivery, previous treatments, the general health and/or age of the subject, and other diseases present. Moreover, treatment of a subject with a therapeutically effective amount of a compound (or combination of agents, e.g., a SIRT1 inhibitor and PPARγ agonist) can include a single treatment or, preferably, can include a series of treatments. Animal models can also be used to determine a useful dose, e.g., an initial dose or a regimen.

In addition, after an administration period described herein with a combination described herein, a maintenance dose can be administered to the subject. For example, the maintenance dose can include a lower dose of one or both of the drugs of the combination described herein, a dose of only one of the drugs described herein (e.g., at the same or at a lower dose than in the initial administration period). As another example, if a combination of a SIRT1 inhibitor and a TZD is used for the initial administration period, a SIRT1 inhibitor or a TZD can be used alone for the maintenance dose. The maintenance dose may be administration of another combination described herein, e.g., a combination described herein but not employed in the initial administration period. For example, if a combination of a SIRT1 inhibitor and a TZD is used for the administration period, a combination of a SIRT1 inhibitor and a non-TZD can be used for the maintenance dose, and vice versa. The maintenance dose can be administered, e.g., for a period of one, two three, four, five, six, seven, eight, nine, ten, eleven, twelve, or more months (e.g., for up to 24 or 36 months or longer) after termination of the initial administration period.

An effective amount of the compound described above lay range from about 0.1 mg/Kg to about 500 mg/Kg, alternatively from about 1 to about 50 mg/Kg. For example, a TZD, such as rosiglitazone, can be administered in doses of 10 mg/kg/day. Further examples of TZD doses include rosiglitazone (≧4 mg/day) or pioglitazone (≧30 mg/day).

Effective doses will o vary depending on t of administration, as well as the co-administration with other agents, e.g., a second agent described herein.

Antibodies

Exemplary agents that inhibit SIRT1 include antibodies that bind to (e.g., inhibit the activity of) SIRT1. In one embodiment, the antibody inhibits the interaction between the protein and its binding partner e.g., an enzyme and its substrate), e.g., by physically blocking the interaction, decreasing the affinity of the protein for its binding partner, disrupting or destabilizing protein complexes, sequestering the protein, or targeting the protein for degradation. In one embodiment, the antibody can bind to the protein at one or more amino acid residues that participate in the binding interface between the protein and its binding partner. Such amino acid residues can be identified, e.g., by alanine scanning. In another embodiment, the antibody can bind to residues that do not participate in the binding. For example, the antibody can alter a conformation of the protein and thereby reduce binding affinity, or the antibody may sterically hinder binding.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FRs and CDRs has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol, 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay (1988) Ann. Rev Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with a target protein, e.g., SIRT1.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can he human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

Antibody Generation,

Antibodies that bind to a target protein (e.g., SIRT1) can be generated by a van of means, including immunization, e.g., using an animal, or in vitro methods such as phage display. All or part of the target protein can be used as an immunogen or as a target for selection. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nat. Gen. 7:13-21; US Published Application No. 2003-0070185; U.S. Pat. No. 5,789,650; and PCT Application WO 96/34096.

Non-human antibodies to the target proteins can also be produced, e.g., in a rodent. The non-human antibody can be humanized, e.g., as described in EP 239 400; U.S. Pat. Nos. 6,602,503; 5,693,761; and 6,407,213, deimmunized, or otherwise modified to make it effectively human.

EP 239 400 (Winter et al.) describes altering antibodies by substitution (within a given variable region) of their complementarily determining regions (CDRs) for one species with those from another. Typically, CDRs of a non-human (e.g., murine) antibody are substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Other methods for humanizing anitbodies can also be used. For example, other methods can account for the three dimensional structure of the antibody, framework positions that are in three dimensional proximity to binding determinants, and immunogenic peptide sequences. See, e,g., PCT Application WO 90/07861: U.S. Pat. Nos: 5,693,762; 5,693,761; 5,585,089; and 5,530,101; Tempest et al, (1991) Biotechnology 9:266-271 and U.S. Pat. No, 6,407,213: Still another method is termed “humaneering” and is described, for example, in US Published Application No, 2005-008625.

Fully human monoclonal antibodies that bind to target proteins can be produced, e.g., using in vitro-primed human splenocytes,as described by Boerner et al, (1991) J. Immunol. 147:86-95. They may be prepared by repertoire cloning as described by Persson et al. (1991) Proc. Nat. Acad. Sci. USA 88:2432-2436 or by Huang and Stollar (1991) J. Immunol. Methods 141:227-236; also U.S. Pat. No. 5,798,230. Large non-immunized human phage display libraries may also be used to isolate high affinity antibodies that can be developed as human therapeutics using standard phage technology (see, e.g., Hoogenboom et al. (1998) Immunotechnology 4:1-20; Hoogenboom et al. (2000) Immunol Today 2:371-378; and US Published Application No. 2003-0232333).

Antibody and Protein Production

Antibodies and other proteins described herein can be produced in prokaryotic and eukaryotic cells. In one embodiment, the antibodies (e.g., scFv's) are expressed in a yeast cell such as Pichia (see, e.g., Powers et al. (2001) J. Immunol. Methods 251:123-35), Hanseula, or Saccharomyces.

Antibodies, particularly full length antibodies, e.g., IgG's, can be produced in mammalian cells. Exemplary mammalian host cells for recombinant expression include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors in y carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216; 4,634,665; and 5,179,017). Exemplary selectable marker genes include the dihydrofolate reductase (DHFR) gene (for use in dhfr host cells with methotrexate selection/amplification) and the nee gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector can also carry a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G.

Antibodies (and Fe fusions) may also include modifications, e.g., modifications that alter Fe function, e.g., to decrease or remove interaction with an Fe receptor or with Clq, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some proteins that include an Fe domain, the antibody/protein production system may be designed to synthesize antibodies or other proteins in which the Fe region is glycosylated. For example, the Fe domain of IgG molecules is glycosylated at asparagine 297 in the CH2 domain. The Fe domain can also include other eukaryotic post-translational modifications. In other cases, the protein is produced in a form that is not glycosylated.

Antibodies and other proteins can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted therein, the protein of interest, e.g., an antibody or. Fc fusion protein. The protein can be purified from the milk, or for some applications, used directly.

Methods described in the context of antibodies can be adapted to other proteins, e.g., Fc fusions and soluble receptor fragments.

Nucleic Acid Agents.

As used herein, an “oligonucleotide agent” refers to a single stranded oligomer or polymer of ribonucleic acid (RNA) or deoxyribonucleic acid (DNA) or both or modifications thereof, which is antisense with respect to its target. This term includes oligonucleotides composed of naturally-occurring nuclcobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

Oligonucleotide agents include both nucleic acid targeting (NAT) oligonucleotide agents and protein targeting (PT) oligonucleotide agents. NAT and PT oligonucleotide agents refer to single stranded oligomers or polymers of ribonucleic acid (RNA) or deoxyribonucleic acid (DNA) or both or modifications thereof. NATs designed to bind to specific RNA or DNA targets have substantial complementarity, e.g., at least 70, 80, 90, or 100% complementary, with at least 10, 20, or 30 or more bases of a target nucleic acid, and include antisense RNAs, microRNAs, antagomirs and other non-duplex structures which can modulate expression. The NAT oligonucleotide agents can target any nucleic acid, e.g., a miRNA, a pre-miRNA, a pre-mRNA, an mRNA, or a DNA: These NAT oligonucleotide agents may or may not bind via Watson-Crick complementarity to their targets. PT oligonucleotide agents bind to protein targets, preferably by virtue of three dimensional interactions, and modulate protein activity. They include decoy RNAs, aptamers, and the like.

Single Stranded Ribonucleid Acid

Oligonucleotide agents include microRNAs (miRNAs). MicroRNAs are small noncoding RNA molecules that are capable of causing post-transcriptional silencing of specific genes in cells such as by the inhibition of translation or through degradation of the targeted mRNA. An miRNA can be completely complementary or can have a region of noncomplementarity with a target nucleic acid, consequently resulting in a “bulge” at the region of noncomplementarity. The region of noncomplementarity (the bulge) can be flanked by regions of sufficient complementarity, preferably complete complementarity to allow duplex formation. Preferably, the regions of complementarity are at least 8 to 10 nucleotides long (e.g., 8, 9, or 10 nucleotides long). A miRNA can inhibit gene expression by repressing translation, such as when the microRNA is not completely complementary to the target nucleic acid, or by causing target RNA degradation, which is believed to occur only when the miRNA binds its target with perfect complementarity. The invention also can include double-stranded precursors of miRNAs that may or may not form a bulge when bound to their targets.

In a preferred embodiment an oligonucleotide agent featured in the invention can target an endogenous miRNA or pre-miRNA. The oligonucleotide agent featured in the invention can include naturally occurring nuclcobases, sugars, and covalent internucleoside (backbone) linkages as well as oligonucleotides having non-naturally-occurring portions that function similarly. Such modified or substituted oligonucleotides are often preferred over native forms because of desirable properties such as, for example, enhanced cellular uptake, enhanced affinity for the endogenous miRNA target, and/or increased stability in the presence of nucleases. An oligonucleotide agent designed to bind to a specific endogenous miRNA has substantial complementarity, e.g., at least 70, 80, 90, or 100% complementary, with at least 10, 20, or 25 or more bases of the target miRNA.

A miRNA or pre-miRNA can be 18-100 nucleotides in length, and more preferably from 18-80 nucleotides in length. Mature miRNAs can have a length of 19-30 nucleotides, preferably 21-25 nucleotides, particularly 21, 22, 23, 24, or 25 nucleotides. MicroRNA precursors can have a length of 70-100 nucleotides and have a hairpin conformation. MicroRNAs can he generated in vivo from pre-miRNAs by enzymes called Dicer and Drosha that specifically process long pre-miRNA into functional miRNA. The microRNAs or precursor mi-RNAs featured in the invention can be synthesized in vivo by a cell-based system or can be chemically synthesized. MicroRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting.

An miRNA or a pre-miRNA can be constructed using chemical synthesis and/or enzymatic ligation reactions using procedures known in the art. For example, an miRNA or a pre-miRNA can be chemically synthesized using naturally occurring nucleotides or variously modified nucleotides designed to increase the biological stability of the molecules or to increase the physical stability of the duplex formed between the miRNA or a pre-miRNA and target nucleic acids, e.g., phosphorothioate derivatives and acridine substituted nucleotides can be used. Other appropriate nucleic acid modifications are described herein. Alternatively, the miRNA or pre-miRNA nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Antisense Type Oligonucleotide Agents

The single-stranded oligonucleotide agents featured in the invention include antisense nucleic acids. An “antisense' nucleic acid includes a nucleotide sequence that is complementary to a “sense” nucleic acid encoding a gene expression product, e.g., complementary to the coding strand of a double-stranded cDNA molecule or complementary to an RNA sequence, e.g., a pre-mRNA, mRNA, miRNA, or pre-miRNA. Accordingly, an antisense nucleic acid can form hydrogen bonds with a sense nucleic acid target.

Given a coding strand sequence (e.g., the sequence of a sense strand of a cDNA molecule), antisense nucleic acids can be designed according to the rules of Watson and Crick base pairing. The antisense nucleic acid molecule can be complementary to a portion of the coding or noncoding region of an RNA, e.g., a pre-mRNA or mRNA. For example, the antisense oligonucleotide can be complementary to the region surrounding the translation start site of a pre-mRNA or mRNA, e.g., the 5′ UTR. An antisense oligonucleotide can be, for example, about 10 to 25 nucleotides in length (e.g., 11, 12, 13, 14, 15, 16, 18, 19, 20, 21, 22, 23, or 24 nucleotides in length). An antisense oligonucleotide can also be complementary to a miRNA or pre-miRNA.

An antisense nucleic acid can be constructed using chemical synthesis and/or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and target nucleic acids, e.g., phosphorothioate derivatives and acridine substituted nucleotides can be used. Other appropriate nucleic acid modifications are described herein. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

An antisense agent can include ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA, and prevent access of the translation machinery to the target RNA transcript, thereby preventing protein synthesis. An antisense molecule including only deoxyribonucleotides, or deoxyribonucleotides and ribonucleotides, e.g., DNA sequence flanked by RNA sequence at the 5′ and 3′ ends of the antisense agent, can hybridize to a complementary RNA, and the RNA target can be subsequently cleaved by an enzyme, e.g., RNAse a Degradation of the target RNA prevents translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

Aptamer-Type Oligonucleotide Agents

An oligonucleotide agent featured in the invention can be an aptamer. An aptamer binds to a non-nucleic acid ligand, such as a small organic molecule or protein, e.g., a transcription or translation factor, and subsequently modifies (e.g., inhibits) activity. An aptamer can fold into a specific structure that directs the recognition of the targeted binding site on the non-nucleic acid ligand. An aptamer can contain any of the modifications described herein.

In one embodiment, an aptamer includes a modification that improves targeting, e.g. a targeting modification described herein.

The chemical modifications described above for miRNAs and antisense RNAs, and described elsewhere herein, are also appropriate for use in decoy nucleic acids.

Exemplary shRNA is include the following sequences:

I. pSUPERretro-SIRT1-RNAi-1 (NM_{—}012238 positions 410):

Target sequence:
CTTGTACGACGAAGACGAC
Forward primer:
GATCCCCCTTGTACGACGAAGACGACTTCAAGAGAGTCGTCTTCGTCGTA
CAAGTTTTTGGAAA
Reverse primer:
AGCTTTTCCAAAAACTTGTACGACGAAGACGACTCTCTTGAAGTCGTCTT
CGTCGTACAAGGGG

II. pSUPERretro-SIRT1-RNAi-2 (NM_{—}012238 positions 589):

Target sequence:
GGCCACGGATAGGTCCATAT
Forward primer:
GATCCCCGGCCACGGATAGGTCCATATTCAAGAGATATGGACCTATCCGT
GGCCTTTTTGGAAA
Reverse primer:
AGCTTTTCCAAAAAGGCCACGGATAGGTCCATATCTCTTGAATATGGACC
TATCCGTGGCCGGG

III. pSUPERretro-SIRT1-RNAi-3 (NM_{—}012238 positions 1091):

Target sequence:
CATAGACACGCTGGAACAG
Forward primer:
GATCCCCCATAGACACGCTGGAACAGTTCAAGAGACTGTTCCAGCGTGTC
TATGTTTTTGGAAA
Reverse primer:
AGCTTTTCCAAAAACATAGACACGCTGGAACAGTCTCTTGAACTGTTCCA
GCGTGTCTATGGGG

Double-Stranded Ribonucleic Acid (dsRNA)

In one embodiment, the invention provides a double-stranded ribonucleic acid (dsRNA) molecule packaged in an association complex, such as a liposome, for inhibiting the expression of a gene in a cell or mammal, wherein the dsRNA comprises an antisense strand comprising a region of complementarity which is complementary to at least a part of an mRNA formed in the expression of the gene, and wherein the region of complementarity is less than 30 nucleotides in length, generally 19-24 nucleotides in length, and wherein said dsRNA, upon contact with a cell expressing said gene, inhibits the expression of said gene by at least 40%. The dsRNA comprises two RNA strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of a gene, the other strand (the sense strand) comprises a region which complementary to the antisense strand, such that the two strands hybridize and form a duplex structure when combined under suitable conditions. Generally, the duplex structure is between 15 and 30, more generally between 18 and 25, yet more generally between 19 and 24, and most generally between 19 and 21 base pairs in length. Similarly, the region of complementarity to the target sequence is between 15 and 30, more generally between 18 and 25, yet more generally between 19 and 24, and most generally between 19 and 21 nucleotides in length. The dsRNA of the invention may further comprise one or more single-stranded nucleotide overhang(s). The dsRNA can be synthesized by standard methods known in the art as further discussed below, e.g., by use of an automated DNA synthesizer, such as are commercially available from, for example, Biosearch, Applied Biosystems, Inc.

The dsRNAs suitable for packaging in the association complexes described herein can include a duplex structure of between 18 and 25 basepairs (e.g., 21 base pairs). In some embodiments, the dsRNAs include at least one strand that is at least 21 nt long. In other embodiments, the dsRNAs include at least one strand that is at least 15, 16, 17, 18, 19, 20, or more contiguous nucleotides.

Sirtuins

Sirtuins are members of the Silent Information Regulator (SIR) family of genes. Exemplary mammalian sirtuins include SIRT1, SIRT2, and SIRT3, e.g., human SIRT1, SIRT2, and SIRT3. A compound (e.g., SIRT inhibitor) described herein may inhibit one or more activities of a mammalian sirtuin, e.g., SIRT1, SIRT2, or SIRT3, e.g., with a Ki of less than 500, 200, 100, 50, or 40 nM. Sirtuins are described in detail, e.g., in US Published Application No, 2006-0074124.

Exemplary compounds described herein may inhibit activity of SIRT1 by at least 10, 20, 25, 30, 50, 80, or 90%, with respect to a natural or artificial substrate described herein. For example, the compounds may have a Ki, of less than 500, 200, 100, or 50 nM.

Kits

The compounds (e.g., a SIRT1 modulator (e.g., inhibitor) and a second agent, e.g., a PPARγ agonist) described herein provided in a kit. The kit includes (a) the compounds described herein, e.g., a composition(s) that includes a compound(s) described herein, and, optionally (b) informational material. The informational material can be descriptive, instructional, marketing or other material that relates to the methods described herein and/or the use of a compound(s) described herein for the methods described herein.

The informational material of the kits is not limited in its form. In one embodiment, the informational material can include information about production of the compound, molecular weight of the compound, concentration, date of expiration, hatch or production site information, and so forth. In one embodiment, the informational material relates to methods for administering the compound.

In one embodiment, the informational material can include instructions to administer a compound(s) (e.g., the combination of a SIRT1 modulator (e.g., inhibitor) and second agent, e.g., a PPARγ agonist) described herein in a suitable manner to perform the methods described herein, e.g., in a suitable dose, dosage form, or mode of administration (e.g., a dose, dosage form, or mode of administration described herein). In another embodiment, the informational material can include instructions to administer a compound(s) described herein to a suitable subject, e.g., a human, e.g., a human having or at risk for a disorder described herein, e.g., cancer, e.g., breast or colon cancer.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound(s) described herein, the composition of the kit can include other ingredients, such as a solvent or buffer, a stabilizer, a preservative, a flavoring agent (e.g., a bitter antagonist or a sweetener a fragrance or other cosmetic ingredient and/or a second agent for treating a condition or disorder described herein. Alternatively, the other ingredients can be included in the kit, but, in different compositions or containers than a compound described herein. In such embodiments, the n include instructions for admixing a compound(s) described herein and the other ingredients, or for using a compound(s) described herein together with the other ingredients, e.g., instructions on combining the two agents prior to administration.

A compound(s) described herein can be provided in any form, e.g., liquid, dried or lyophilized form. It is preferred that a compound(s) described herein be substantially pure and/or sterile. When a compound(s) d described herein is provided in a liquid solution, the liquid solution preferably is an aqueous solution, with a sterile aqueous solution being preferred. When a compound(s) described herein is provided as a dried form, reconstitution generally is by the addition of a suitable solvent. The solvent, e.g., sterile water or buffer, can optionally be provided in the kit.

The kit can include one or more containers for the composition containing a compound(s) described herein. In some embodiments, the kit contains separate containers (e.g., two separate containers for the two agents), dividers or compartments for the composition(s) and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e,g., packaged for surgical insertion.

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### Examples

The examples demonstrate the use of inhibitors of the NAD+-dependent protein deacetylase, SIRT1, as therapeutics to combat metabolic disorders including insulin resistance, type 2 diabetes, cardiovascular and inflammatory diseases. The use of these compounds is based on results from recent investigations showing that inhibition of SIRT1 activity in fat cells leads to an increase in production of proteins (including adiponectin and fibroblast growth factor 21) known to sensitize individuals to insulin and also overcome certain metabolic disorders. Present drugs used to treat these disorders include synthetic ligands (thiazolidinediones) of the nuclear receptor, peroxisome proliferators-activated receptor gamma (PPARgamma; PPARg; PPARγ). There are side effects associated with use of thiazolidinediones including significant weight gain and water retention (edema). Inhibition of SIRT1 activity induces changes in the production of adipocyte proteins similar to those induced by treatment with PPARgamma ligands.

The nuclear receptor, PPARgamma, regulates a plethora of functions in metabolic tissues most notably adipose tissue. A principal function of PPARgamma is to promote the formation of fat cells and also to regulate many metabolic processes once the mature fat cell has developed. Obese individuals become insulin resistant, develop type 2 diabetes and cardiovascular disease as a result of increased circulating amounts of lipids and a reduction in the amount of circulating hormones that promote clearance of lipids and glucose from the blood stream. These obesity-related defects result from the development of an inflammatory response in adipose tissue as individuals gain weight. Production of inflammatory cytokines or necrosis factor alpha (TNFalpha) by macrophages in obese adipose depots the normal functions of the adipocyte. Previous studies have shown that activation of PPARgamma with synthetic ligands such as specific thiazolidinediones reduces the response of adipocytes to the inflammatory agents and leads to a “healthier” fat cell functioning to secrete insulin-sensitizing adipokines such as adiponectin and enhance the uptake of circulating glucose and lipids. Consumption of thiazolidinediones by insulin-resistant individuals also leads to several side effects most notably retention of water due to the action of these compounds in the kidney. Consequently, alternative mechanisms to promote healthier adipocytes including enhanced secretion of natural insulin sensitizers would be advantageous. Investigations by the inventors have shown that inhibition of the NAD+-dependent protein deacetylase, SIRT1, enhances the production of two natural insulin sensitizers, adiponectin and fibroblast growth factor 21 (FGF21), from adipocytes as well as increase expression of a group of genes that are also enhanced by treatment of fat cells with thiazolidinediones. The inventors propose that small compound inhibitors of SIRT1 are effective therapeutics for the treatment of obesity-related disorders including type 2 diabetes and cardiovascular disease. Since SIRT1 regulates PPARgamma activity it is likely that the inhibitors of SIRT1 will also have use in treating other disorders shown to be regulated by PPARgamma including other inflammatory responses such as Crohn's disease and related diseases, treatment of certain cancers such as breast and prostate. SIRT1 also affects expression of hypoxia-induced genes, therefore, the inhibitors might also been used to treate hypoxia-associated diseases.

### Example 1

Adiponectin is secreted from adipose tissue in response to metabolic effectors in order to sensitize the liver and muscle to insulin. Reduced circulating levels of adiponectin that usually accompany obesity contribute to the associated insulin resistance. The molecular mechanisms controlling the production of adiponectin are essentially unknown. In this report, we demonstrate that the ER oxidoreductase Ero1-Lα and effectors modulating PPARγ and SIRT1 activity regulate secretion of adiponectin from 3T3-L1 adipocytes. Specifically, adiponectin secretion and Ero1-Lα expression are induced during the early phase of adipogenesis, but are then down-regulated during the terminal phase, coincident with an increased expression of SIRT1. Suppression of SIRT1 or activation of PPARγ enhances Ero1-Lα expression and stimulates secretion of high molecular weight (HMW) complexes of adiponectin in mature adipocytes. Suppression of Ero1-Lα through expression of a corresponding siRNA reduces adiponectin secretion during the differentiation of 37123-L1 preadipocytes. Moreover, ectopic expression of Ero1-Lα in Ero1-Lα-deficient 3T3 fibroblasts stimulates the secretion of adiponectin following their conversion into adipocytes and prevents the suppression of adiponectin secretion in response to activation of SIRT1 by exposure to resveratrol. These findings provide a framework to understand the mechanisms by which adipocytes regulate secretion of adiponectin in response to varying metabolic states.

Introduction

The rapid rise in the prevalence of obesity is a major factor contributing to the dramatic increase in insulin resistance and type 2 diabetes among the young as well as the elderly in western society (22). Understanding the link between adiposity and glucose homeostasis is the focus of many investigations. Recent studies have identified the adipocyte as an endocrine cell that functions not only to store and metabolize lipids but also to secrete a plethora of biologically active molecules (adipokines) that participate in overall energy balance (19). Notable among these adipokines is adiponectin, which has been shown to play an important role in regulating insulin control of glucose metabolism and whose secretion from the adipocyte is modulated in response to varying metabolic states (17, 35). Specifically, circulating adiponectin levels are decreased in obese and insulin-resistant subjects and many clinical investigations support the notion that metabolic suppression of adiponectin production in obese individuals contributes significantly to the metabolic syndrome, including insulin resistance, atherosclerosis and hypertension (35).

Adiponectin is synthesized as a single polypeptide of 30 kD and is then assembled in the endoplasmic reticulum into higher molecular weight complexes prior to its secretion. Circulating adiponectin consists of an array of complexes composed of multimers of the 30 kD polypeptide facilitated by distinct disulfide bonds generating trimers, middle molecular weight hexamers (MMW) and an elaborate high molecular weight complex (HMW) that has recently been suggested to possess the most potent insulin sensitizing activity of all the complexes (18, 25, 9). In fact, it appears that the ratio of HMW adiponectin to other complexes changes in direct response to perturbations in metabolic state accompanying obesity and its associated disorders (25, 37). It is important, therefore, to define the molecular mechanisms controlling production of HMW adiponectin and determine how these processes respond to changes in metabolism.

Recent investigations suggest that a major mode of action of the thiazolidinedione (TZDs) class of insulin-sensitizing drugs is to increase the circulating levels of HMW adiponectin (18, 25). Since TZDs are also potent ligands for the nuclear receptor PPARγ, these observations support a role for PPARγ in regulating adiponectin production. In fact, studies by Shimomura and colleagues (16) have provided evidence that PPARγ can regulate expression of adiponectin by promoting transcription of the corresponding mRNA through a PPARγ response element in the promoter of its gene. A recent study, however, states that increased plasma adiponectin in response to the TZD pioglitazone does not result from increased gene expression (29). Additional mechanisms must, therefore, be operating; some of which might involve a PPARγ-dependent expression of proteins participating in the formation and secretion of HMW adiponectin. Furthermore, PPARγ might also function to mediate the response of adipocytes to metabolic perturbations associated with obesity and insulin resistance. In this regard, the NAD-dependent deacetylase, SIRT1, has recently been shown to suppress PPARγ activity in response to calorie restriction in fasted animals leading to fat mobilization in adipose depots (26). In the present study, we questioned whether changes in metabolites, such as glucose, regulate secretion of HMW adiponectin through the expression of a component of the secretory process whose production is controlled by SIRT1 modulation of PPARγ activity.

Formation of disulfide bonds occurs in the lumen of the ER by at least two pathways that oxidize cysteine pairs to form native bonds as well as isomerization of non-native disulfide bonds (32). The major pathway is comprised principally of the flavoprotein Ero1 and members of the protein disulfide isomerase (PDI) family. PDI is a multi-domain member of the thioredoxin superfamily that can catalyse thiol-disulfide oxidation, reduction and isomerization that facilitate the formation of intra—as well as inter-molecular disulfide bonds (10). Ero1 is an ER membrane-associated oxidoreductase that utilizes the oxidizing power of oxygen to generate disulfide bonds in itself, which it then transfers to PD1 (12, 13, 27). Oxidized PD1 is then able to transfer its disulfide bonds to appropriate substrates. The entire process consists, therefore, of transmission of oxidizing equivalents between Ero1, PD1 and secretory proteins that involve a series of direct thiol-disulfide exchange reactions between each of the proteins (8, 11, 36). Mammals express two related Ero1 proteins, Ero1-Lα and Ero1-lβ. Ero1-Lβ is produced primarily in secretory cells and its expression is induced by the unfolded protein response (9, 24). In contrast, Ero1-Lα is expressed in most cell types where it is considered to be the rate-limiting step in disulfide bond formation (4, 24).

In the following studies, we demonstrate that secretion of HMW adiponectin from 3T3-L1 adipocytes is regulated through the nutrient control of SIRT1 activity. Inhibition of SIRT1 and/or activation of PPARγ lead to increased expression of Ero1-Lα and a corresponding increase in HMW adiponectin secretion. Suppression of Ero1-Lα through expression of a corresponding siRNA reduces adiponectin secretion during the differentiation of 3T3-L1 preadipocytes. Moreover, ectopic expression of Ero1-Lα in 3T3 fibroblasts stimulates the secretion of adiponectin during their differentiation into adipocytes. These data suggest that nutrient control of adiponectin secretion is mediated through SIRT1-associated regulation of the PPARγ-responsive gene Ero1-Lα.

Materials and Methods

Materials: Dulbecco's modified eagle's medium (DMEM) with and without 4.5 g/L glucose was purchased from Mediatech, Inc. (Herndon, Va.), fetal bovine serum (FBS) from Gemini Bio-Products, calf serum and TRIzol from Invitrogen and D-Glucose from American Bioanalytical (Natick, Mass.). The PPARγ agonist troglitazone was obtained from Biomol International while the PPARγ antagonist T0070907 was purchased from Tom,. Resveratrol was from Cayman Chemical and lactate, pyruvate, nicotinamide were all from Sigma. All other chemicals were supplied by American Bioanalytical.

Cell Culture: Murine 3T3-L1 preadipocytes were cultured and maintained in DMEM supplemented with 0% calf serum. Differentiation was induced by exposure of postconfluent cells to DMEM containing 10% MS, 1 μM dexamethasone (DEX), 0.5 mM 3-isobutyl-1-methylxanthine (MIX) and 1,61 μM insulin. After 48 hours, the medium was changed to DMEM containing 10% FBS every two days. Human embryonic kidney (HEK)-293T cells were cultured in DMEM with 10% FBS; The Swiss F-PPARγ cells were differentiated as recently described (Wang et al, 2006; accompanying paper).

Plasmid Construction and Retrovirus Transfection/Infection: A PCR fragment corresponding to the coding region of murine Ero1-Lα messenger RNA was generated using 3T3 L1 adipocyte cDNA as template and the following oligonucleotides also containing either a BamHI or a SalI restriction site as primers: 5′-GAA GGA TCC ATG GGC CGC GCC TGG GGC TTG CTC GTT-3′ (sense) and 5′-CGC CGT CGA CGG CAC ATT CCA ACC GTC CTC CTC AGT G-3′(antisense). The PCR product was subcloned into the multi-cloning site of pRevTRE retrovirus using BamHI and Sal I restriction endonucleases. pSUPER-SIRT1 siRNA retroviral vector was generously provided by Dr Jim Xiao of Boston University School of Medicine and consisted of the plasmid recently described (26). HEK-293T cells were grown to 70% confluence in 100 mm-diameter dishes at which stage they were transfected with the DNA-FUGENE cocktail consisting of 36 μL Eugene 6, 6 μg retrovirus plasmid, 6 μg pVPack-VSV-G vector, 6 μg pVPack-GAG-POL vector and 164 μL DMEM without FBS. Twenty-four hours later, the medium was replaced with 6 mL fresh DMEM containing 10% FBS. One day after that, the culture medium containing high-titer retrovirus was harvested and filtered through a 0.45 μm pore size filter. The viral filtrate was used to infect both 3T3-L1 preadipocytes and Swiss 3T3 fibroblasts.

Transient Knockdown of Ero1-Lα, in 3T3-L1 Λdipoecytes.

3T3-L1 preadipocytes were cultured in 60 mm dishes and induced to differentiate as described above. On day 4, the differentiation-inducing medium was replaced with 2.5 mL, fresh DMEM containing 10% FBS. 15 μL TransIT-TKO Transfection Reagent (Mirus Bio Cooperadon. Madison, Wis.) was diluted into 250 μL serum free DMEM and incubated at room temperature for 20 minutes, at which time 100 nM Ero1-Lα RNAi duplex or control duplex Oil in the dish) was added and mixed thoroughly. After incubation at room temperature for another 20 minutes, the resulting TranIT-TKO/RNAi mixture was gently mixed into the adipocyte cultures. The next day, the medium was changed to 2.5 mL flesh DMEM containing 10% FBS and the transfection was repeated as on day 4. On day 6, the total cellular proteins were harvested and subjected to Western blot analysis. The modified synthetic Ero1-Lα RNAi duplexes (Stealth™ siRNA) were from Invitrogen Life Technologies. The sequences are as follows:

#1, GCUGAGUAUGUGGACUUACUCCUUA;
#2, GGGCACUGCUCUGAAGAUCUUGUUU;
#3, GGGCUCUCUCCAAAGUGCUUCCAUU.

A GFP RNAi duplex was used as the negative control.

A Analysis: Total RNA was isolated using TRIzol following the manufacturer's instructions and was subjected to RT-PCR analysis as outlined in the Promega product manual as previously described (31). Primers used for the RT-PCR analysis were as follows: PPARγ, 5′-CCA GAG CAT GGT GCC TTC GCT G-3′and 5′-GAG CTG ACC CAA TGG TTG CTG-3′; adiponectin, 5′-ACT CCT GGA GAG AAG GAG AA-3′ and 5′-TTG TCC TTC TTG AAG AGG CTC ACC-3′. In the case of analyzing Ero1-Lα mRNA, primers were the same as those listed above for generating the Ero1-Lα pRevTRE expression vector.

Western Blot Analysis of Proteins: Isolation and western blot analysis of total cell proteins was performed as outlined previously (21). The antibodies employed in the analysis were as follows: Mouse polyclonal anti-adiponectin antibody (Affinity BioREagents, Golden, Colo.), polyclonal anti-SIRT1 antibody (Upstate), anti-PPARγ and anti-C/EBPα (Santa Cruz Biotechnology), anti-Ero1 polyclonal antibody (Abnova Co., Taiwan, China) and polyclonal anti-aP2 serum (Dr. David Benlohr at University of Minnesota). For analysis of secreted proteins, aliquots of the culture medium were centrifuged at low speed to remove debris and then subjected to standard SDS-PAGE as above or non-reducing SDS-PAGE as outlined by Kadowaki and collaborators (37). Prior to electrophoresis, the samples were mixed with 4× non-reducing protein sample buffer (200 mM, Tris pII-16.8, 8% SDS, 0.4% bromophenot blue, 40% glycerol) and incubated at room temperature for one hour. Care was taken to ensure that all components of the SDs PAGE system were completely free of reducing agents.

All experiments were performed at least three tiles and figures presented are representative of the data.

Results

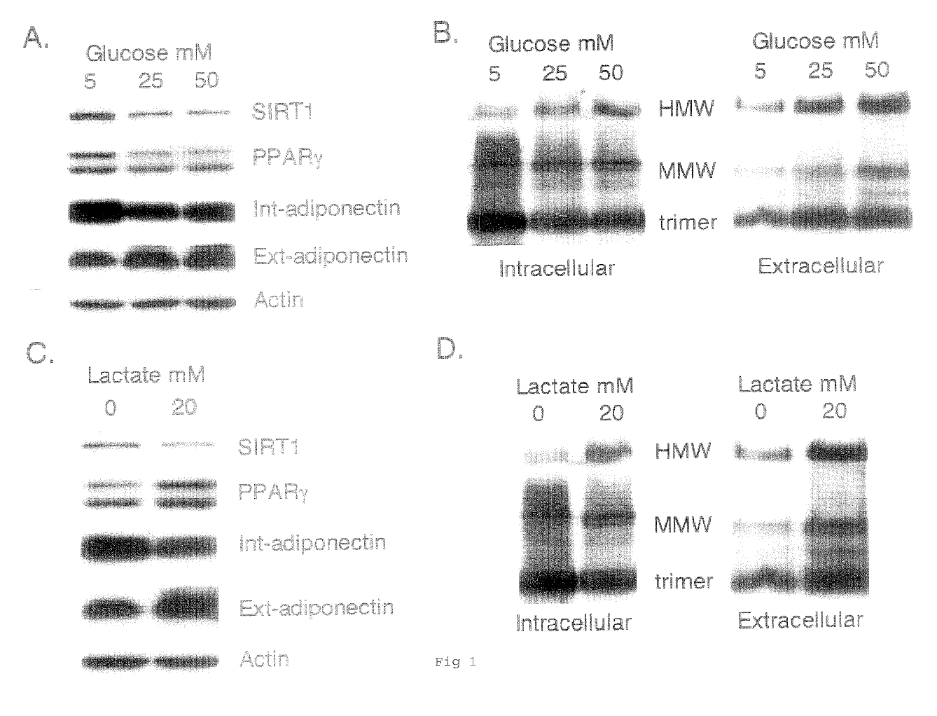

To gain insight into mechanisms by which nutrients might regulate adiponectin synthesis and secretion, we exposed 3T3-L1 adipocytes to increasing doses of either glucose or lactate for 72 hrs and analyzed adiponectin production on western blots. FIG. 1A shows that exposing 3T3-L1 adipocytes to increasing concentrations of glucose dramatically increases the amount of adiponectin secreted into the medium (Ext) whilst decreasing the level of intracellular adiponectin (Int). We also measured the abundance of the NAD-dependent deacetylase, SIRT1, since its expression and activity has been shown to be regulated by nutrients, such as glucose and lactate, that influence the ratio of NAD⁺/NADH (30). In fact, there is a significant decrease of SIRT1 expression in response to an increase in glucose concentration.

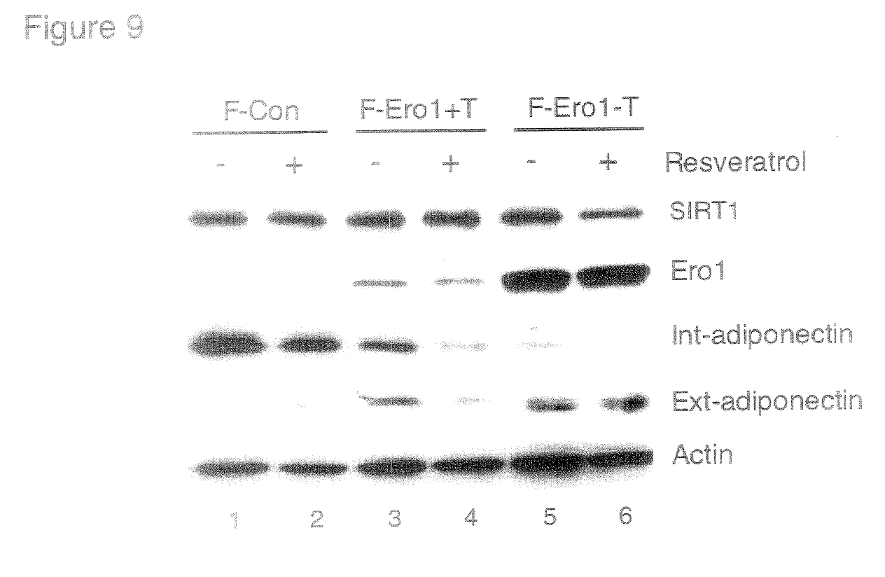

The secreted adiponectin detected by western blot analysis of SDS-PAGE performed under reducing conditions as shown in FIG. 1A corresponds to the monomer polypeptide of MW 30 kD. Recent studies have demonstrated that adiponectin is processed in the endoplasmic reticulum prior to secretion into an elaborate set of higher ordered structures involving disulfide-bond linkage of each of the monomers into HMW complexes composed of several hexamers. In addition, investigations have also shown that the HMW forms of adiponectin possess the highest insulin sensitizing activity. It is important, therefore, to identify the specific forms of adiponectin that are secreted from 3T3-L1 adipocytes in response to various effectors. Recent studies have shown that analysis of adiponectin on non-reducing SDS-PAGE faithfully represents the complexity of the secreted adiponectin molecules produced in adipose tissue and present in the circulation (37). FIG. 10 shows the separation of the various forms of adiponectin secreted into the medium of cultures of 3T3-L1 preadipocytes following non-reducing SDS PAGE under conditions that preserve the disulphide bonds.

To assess the effect of glucose on the complexity of the secreted adiponectin, intracellular and extracellular protein samples were subjected to non-reducing SDS-PAGE without heat. FIG. 1B demonstrates a significant increase in the appearance of the HMW species in the media in response to glucose, with a corresponding decrease in the abundance of the trimer within the cell. Mature adipocytes rapidly metabolize glucose to pyruvate during glycolysis. Lactate is also converted to pyruvate through the action of lactate dehydrogenase with conversion of NAD+ to NADH. It is encouraging; therefore, that lactate has a similar effect on enhancing the secretion of HMW forms of adiponectin at the expense of a decrease in intracellular trimers (FIGS. 1C and 1D).

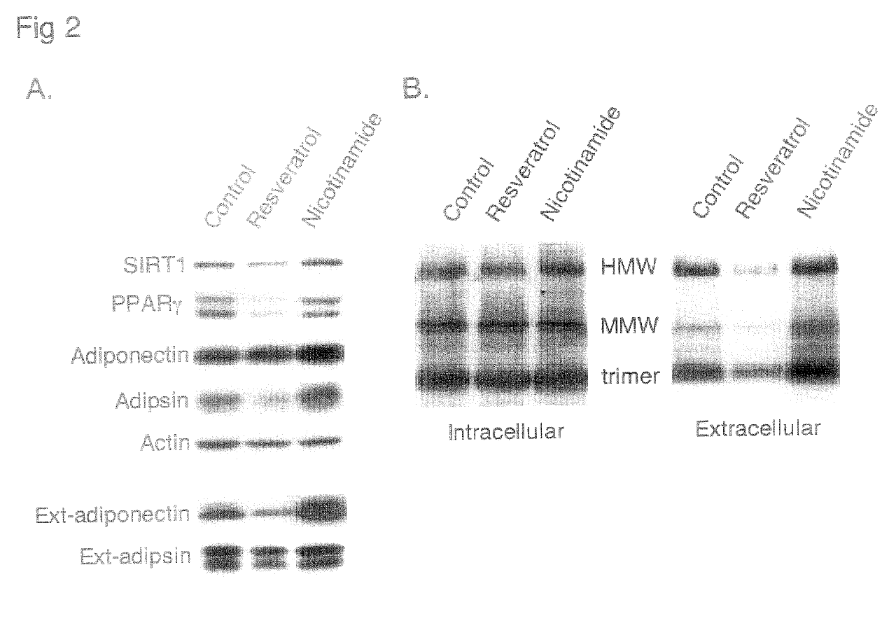

Calorie restriction (low glucose) enhances the activity of the NAD-dependent deacetylase SIRT1 due to an increase in NAD^{|}/NADH ratio. Consequently, we questioned whether the effect of glucose and lactate on adiponectin secretion involved SIRT1. 3T3-L1 adipocytes were, therefore, exposed to an activator (resveratrol) or inhibitor (nicotinamide) of SIRT1 activity. FIG. 2A demonstrates that activation of SIRT1 reduces the secretion of adiponectin (Ext) without significantly affecting synthesis of the protein (Int) or without affecting secretion of adipsin. In contrast, inhibition of SIRT1 has the opposite effect by enhancing secretion of adiponectin that is many times greater than the slight increase in the production of the protein and has no effect on adipsin. Furthermore, modulation of SIRT1 activity leads to a corresponding change in the relative abundance of the different adiponectin complexes. Specifically, resveratrol decreases whereas nicotinamide enhances secretion of the HMW complexes (FIG. 2B).

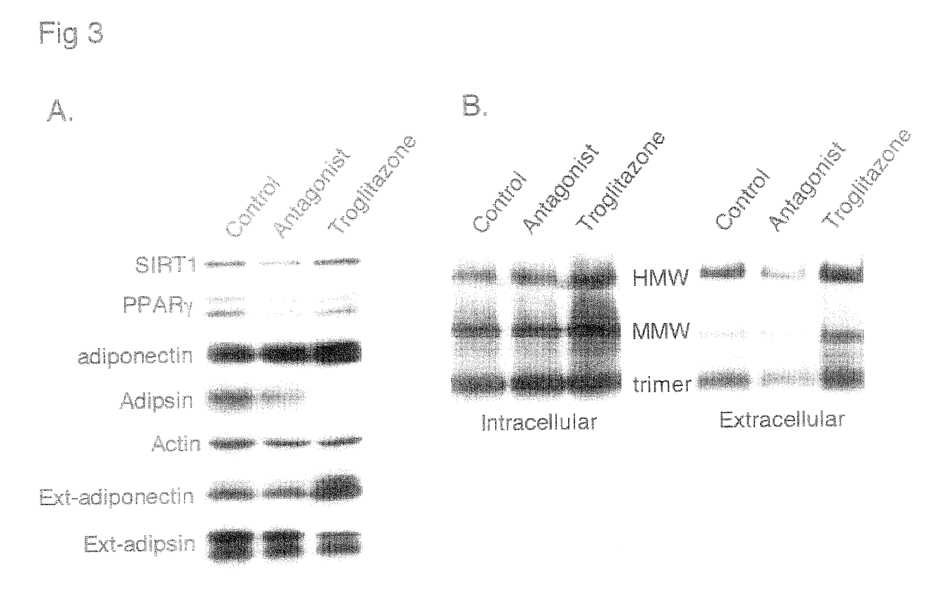

SIRT1 can regulate PPARγ activity, we questioned, therefore, whether PPARγ was also involved in controlling adiponectin secretion. Treatment of 3T3-L1 adipocytes with the PPARγ, ligand, troglitazone, significantly increases the amount of secreted HMW adiponectin and attenuates secretion of adipsin, whereas T0070907, a PPARγ antagonist, has the opposite effect (FIGS. 3A and 3B). Interestingly, activation of PPARγ induces expression of SIRT1 (FIG. 3A) consistent with the fact that SIRT1 expression is enhanced during adipogenesis (26).

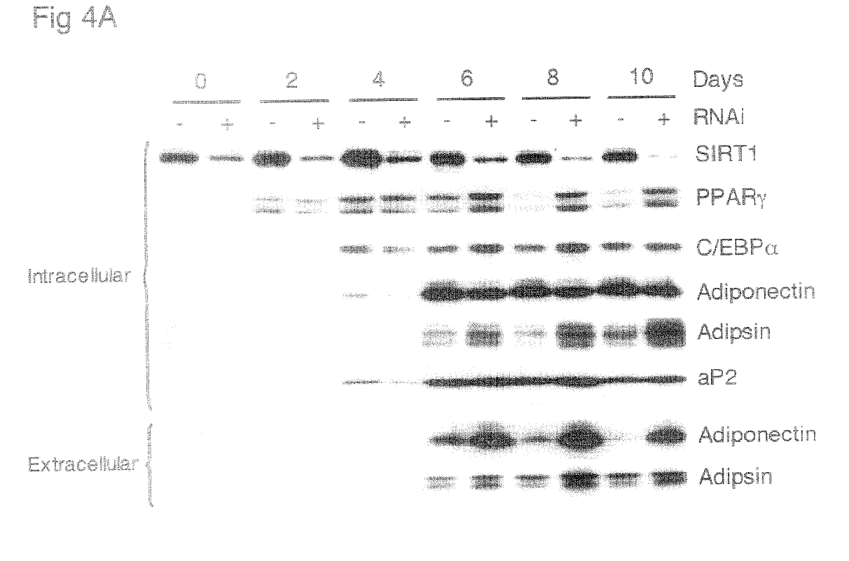

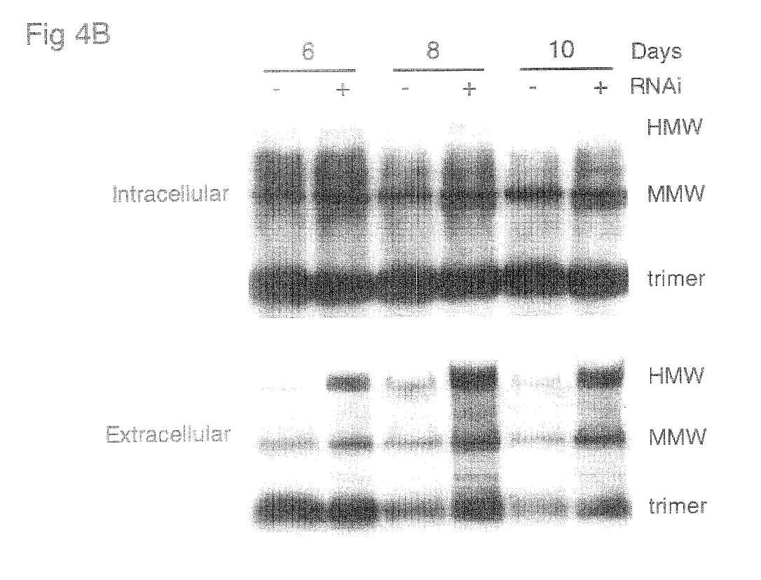

To confirm that SIRT1 regulates adiponectin secretion, we generated a 3T3-L1 cell line in which SIRT1 was “knocked down” using RNAi technology. Western blot analysis of proteins extracted from vector control cells and SIRT1 knock down cells at different times during their differentiation into adipocytes demonstrates an extensive increase in SIRT1 expression at 2-4 days of differentiation of control cells. In contrast, the SIRT1 RNAi effectively down-regulated SIRT1 expression in the knock down cells and in so doing prevented the decrease in PPARγ expression during terminal adipogenesis. More importantly, secretion of adiponectin (extracellular) decreased significantly during adipogenesis in control cells in a manner coincident with the increase in SIRT1 and decrease in PPARγ (FIG. 4A, minus lanes). In contrast, knock down of SIRT1 resulted in high levels of adiponectin secretion that consisted of abundant amounts of HMW species (FIG. 4B). There was no dramatic effect of suppression of SIRT1 on secretion of adipsin or on the production of adiponectin (intracellular), but there was a slight enhancement of synthesis of the fatty acid binding protein 4 (aP2) and adipsin (FIG. 4A).

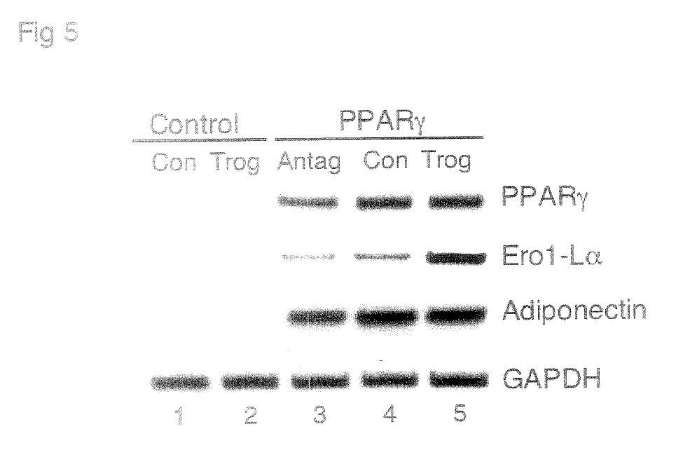

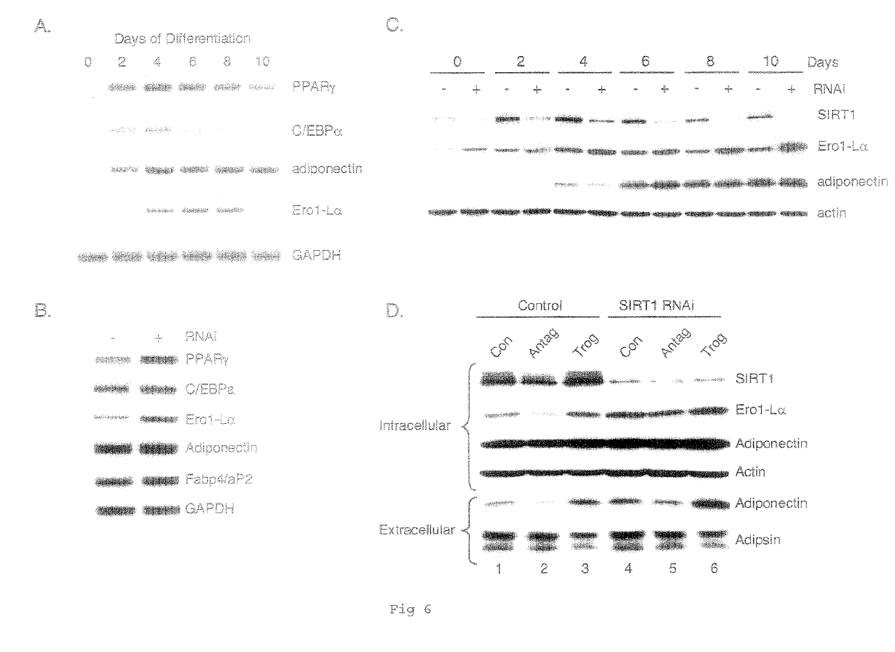

Together, these data suggest that SIRT1 and PPARγ are regulating a component of the machinery involved in the processing and secretion of adiponectin. Recent studies have identified an elaborate process in which a series of endoplasmic reticulum proteins including the oxidoreductase Ero1-Lα and protein disulfide isomerase regulate secretion of a variety of molecules. In fact, investigations by others (38) have show that Ero1-Lα mRNA expression is enhanced during adipogenesis in 3T3-L1 preadipocytes; consequently, we questioned whether the Ero 1-Lα gene is a direct target of PPARγ and whether Ero1-Lα regulates secretion of adiponectin. To address the first question, we ectopically expressed PPARγ in Swiss 3T3 fibroblasts to create a cell line referred to as WT.-PPARγ cells which is capable of undergoing complete conversion into adipocytes in response to inducers of adipogenesis including dexamethasone (D), isobutylmethylxanthine (M) and insulin (I) with or without troglitazone. FIG. 5 demonstrates that the ectopic PPARγ induces expression of Ero1-Lα mRNA in these cells in response to DMI alone to levels that are significantly higher than those expressed in control cells containing the empty retroviral vector (FIG. 5, compare lane 4 with lane 1). Furthermore, the expression of Ero1-Lα mRNA is enhanced several fold by exposure of the PPARγ cells to troglitazone with no apparent increase in adiponectin expression (FIG. 5, compare lane 5 with lane 4). Similarly, inhibition of PPARγ activity by the antagonist T0070907, attenuates Ero1-Lα expression (FIG. 5., compare lane 3 with lane 4). These data arc cot ant, therefore, notion that Ero1-Lα gene expression is directly responsive to the action of PPARγ. As stated above, other investigators have shown that expression of Ero1-Lα is regulated during adipogenesis and the data in FIG. 6A are consistent with those studies since there is an extensive induction of Ero1-Lα mRNA expression in differentiating 3T3-L1 preadipocytes at a time coinciding with the increase in PPARγ activity (day 2-4). Interestingly, its expression declines significantly during the latter stages of differentiation similar to that observed for adiponectin secretion (FIG. 4A). To determine whether this decrease in Ero1-Lα expression is due to enhanced SIRT1 activity, Ero1-Lα mRNA was analyzed in SIRT1 knock down cells. FIGS. 6B and 6C show that levels of Ero1-Lα mRNA and protein are much higher in the knock down cells compared to vector controls. In addition, FIG. 6C shows the decrease in Ero1-Lα during adipogenesis in control cells that is consistent with the drop in corresponding mRNA levels (FIG. 6A).

It is very likely that SIRT1 is suppressing adiponectin secretion by suppressing expression of the PPARγ-responsive gene, Ero1-Lα. The data in FIG. 6D are consistent with this notion, since the effect of knocking down SIRT1 in combination with exposure to troglitazone (lane 4) significantly enhances Ero1-Lα expression and adiponectin secretion (extracellular) compared to the modest increase in adiponectin production (intracellular) (FIG. 6D, compare lane 6 with lane 1). It is important to mention that there is no significant effect of SIRT1 knockdown on adipsin secretion (extracellular), but treatment with troglitazone in both cell lines causes a slight decrease in production of adipsin. Additionally, inhibition of PEARγ activity by treatment with the PPARγ antagonist T0070907 inhibits Ero1-Lα expression and suppresses adiponectin secretion in both control and SIRT1 knock down cells (FIG. 6D, compare lanes 2 and as well as 5 and 4).

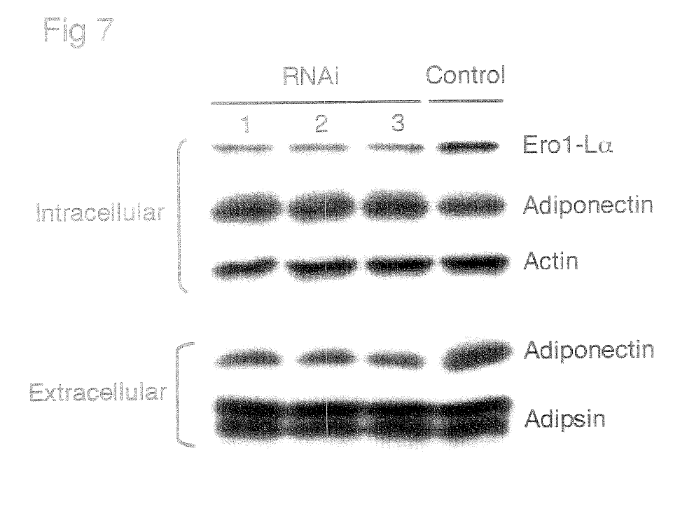

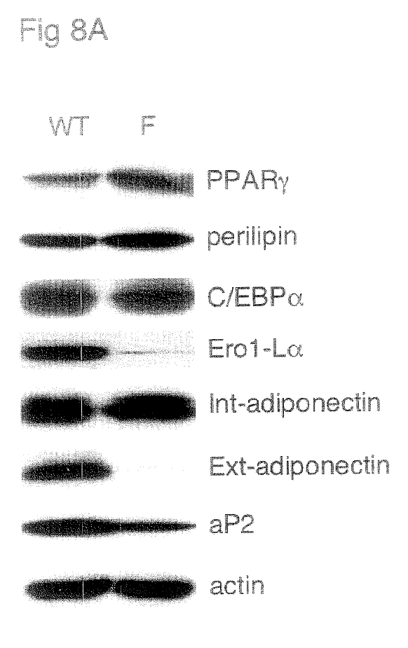

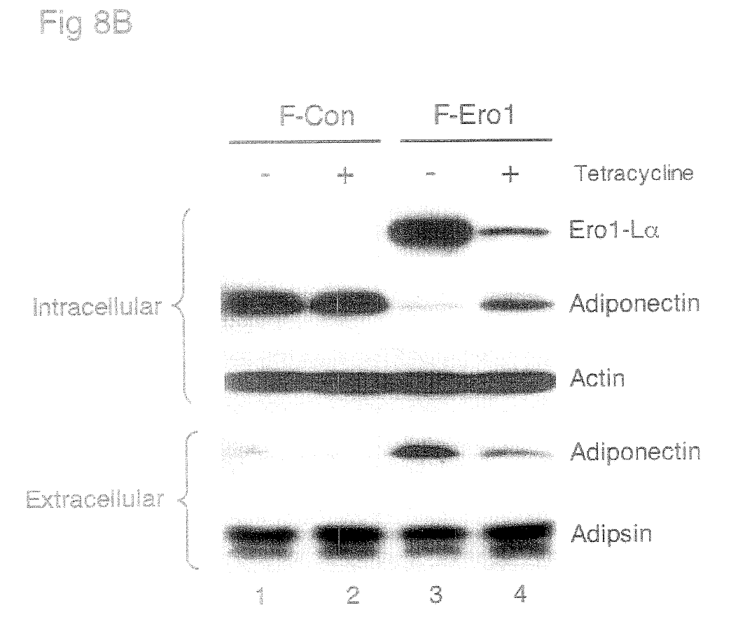

To definitively show a role for Ero1-Lα in controlling adiponectin secretion, we suppressed its expression during the terminal phase of adipogenesis in 3T3-L1 preadipocytes through the transient expression of corresponding siRNAs. This was achieved by transfecting cultures of preadipocytes with three separate siRNAs at day 4 of their differentiation into adipocytes. Cells were then harvested at day 6 for analysis of expression of select proteins as well as secretion of adiponectin. FIG. 7 demonstrates that all three siRNAs attenuate Ero1-lα expression ˜50% during this 2. day period with siRNA #3 being the most effective. The reduction in Ero1-Lα expression had no significant effect on synthesis of adiponectin or actin or secretion of adipsin, but it did significantly reduce the amount of adiponectin secreted from the cells (FIG. 7, compare lanes 1-3 with lane 4). In addition to suppression of Ero1-Lα, we also investigated the effect of overexpressing the protein in cells in which adiponectin secretion and Ero1-Lα expression have been compromised. The data in FIG. 5 shows that ectopic expression of a wild type PPARγ in Swiss fibroblasts (Swiss-WT.-PPARγ cells) induces adipogenesis, which includes expression of Ero1-Lα. In contrast, expression of a PPARγ molecule in which F372 has been modified to alanine (21) (Swiss-PPARγF372A) are capable of undergoing conversion into adipocytes in the presence of troglitazone, but their level of secretion of adiponectin (extracellular) is significantly reduced, relative to the total amount of adiponectin synthesized (intracellular) (FIG. 8A). Additionally, Ero1-Lα expression is virtually undetectable in the Swiss-PPARγF372A adipocytes (F) compared to the amount produced in Swiss-WT.-PPARγ adipocytes (WT.) (FIG. 8A). To test the function of Ero1-Lα, we stably introduced Ero1-Lα cDNA into the Swiss-PPARγF372A cells using a pREV-TET-Ero1-Lα retrovirus that generated a cell line in which we could control the level of Ero1-Lα expression by exposure of the cells to varying concentrations of tetracycline. FIG. 8B shows that conversion of the Swiss-PPARγF372A fibroblasts expressing a pREV-TET vector alone (F-Con) into adipocytes induces synthesis of adiponectin (Intracellular) without any apparent production of Ero1-Lα or secretion of adiponectin (Extracellular) (FIG. 8B, lanes 1 and 2). In contrast, conditional ectopic expression of Ero1-Lα in Swiss-PPARγF372A cells (F-Ero1) leads to abundant production of Ero1-Lα and a corresponding increase in the secretion of adiponectin (Extracellular) (FIG. 8B, lanes 3 and 4). We additionally analyzed adiponectin under non-reducing conditions to determine the effect of ectopic Ero1-Lα on formation of the higher-ordered complexes. FIG. 8C, lane 1 shows the production of the three major complexes of adiponectin (trimer, MMW and HMW) by Swiss adipocytes expressing a WT.-PPARγ similar to the complexes produced by 3T3-L1 adipocytes (see FIG. 1B). Furthermore, these Swiss-WT.-PPARγ adipocytes are able to secrete these adiponectin complexes into the extracellular medium (FIG. 8C, lane 5). Swiss cells expressing mutant PPARγF372A (F-Con), however, produce adiponectin complexes (FIG. 8C, lane 2), but they remain within the cell and are not secreted into the medium (FIG. 8C, lane 6). It is relevant that the complexes produced in the F-Con cells differ somewhat from those in the WT cells since the relative abundance of the MMW and HMW species is very low in the F-Con cell extracts (FIG. 8C, compare lane 2 with lane 1). Interestingly, ectopic expression of Ero1-Lα in the Swiss-PPARγF372A (F-Ero 1)cells leads to a significant increase in the production of MMW and HMW complexes in the cells (FIG. 8C, lanes 3 and 4); but, more importantly, it leads to secretion of abundant amounts of all three adiponectin complexes (trimer, MMW and HMW) into the medium (FIG. 8C, lanes 7 and 8).

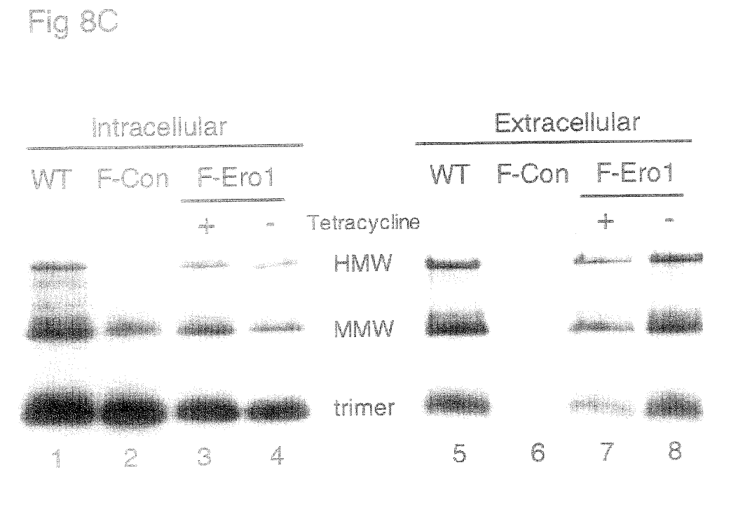

These data demonstrate a definite role for Ero1-Lα in promoting the secretion of HMW adiponectin from adipocytes and suggest that Ero1-Lα likely mediates the effects of metabolic perturbations on adiponectin secretion. To begin to test this notion, we questioned whether ectopic expression of Ero1-Lα could overcome the negative effects of enhanced SIRT1 activity on adiponectin secretion. Consequently, Swiss-F-PPARγ cells expressing either a control vector (F-Con) or ectopic Ero1-Lα (F-Ero1) were induced to differentiate into adipocytes in the presence or absence of tetracycline (+ or −T) to facilitate control of the ectopic Ero1-Lα. At day 4, cells were treated with or without resveratrol at concentrations known to activate SIRT1. FIG. 9 shows that ectopic expression of Ero1-Lα significantly enhances secretion of adiponectin (compare lane 3 with lane 1). This effect is increased more when cells are cultured in the absence of tetracycline, which also increases the ectopic production of Ero1-Lα (compare lane 5 with lane 3). Interestingly, activation of SIRT1 by treatment with resveratrol inhibits secretion of adiponectin in F-Ero1 cells -E-tetracycline consistent with the data in FIG. 2, which shows that resveratrol attenuates adiponectin secretion in 3T3-L1 adipocytes. Furthermore, enhanced production of ectopic Ero1-Lα in the F-Ero1 cells by culture in the absence of tetracycline overcomes the Sirt1-associated inhibition of adiponectin secretion (FIG. 9, compare lanes 4 and 6).

Discussion

Studies during the last few years have clearly shown that circulating adiponectin plays a direct role in sensitizing both liver and muscle to insulin, thus contributing directly to overall glucose homeostasis (18, 35). Moreover, additional studies have identified the HMW complexes of circulating adiponectin as principal mediators of its insulin-sensitizing activity (25, 37). Consequently, we considered it important to identify the effectors and mechanisms controlling the production and secretion of HMW adiponectin from the adipocyte. To this end, the present data show that exposing 3T3-L1 S adipocytes to increasing doses of either glucose or lactate significantly enhances the secretion of HMW complexes of adiponectin without having a significant effect on synthesis of the monomeric protein. Since glucose and lactate are metabolized by the adipocyte to pyruvate, converting NAD⁺ into NADH, we questioned whether the NAD-dependent deacetylase SIRT1 was involved in facilitating this response. The data show that inhibition of SIRT1 activity, by exposure to nicotinamide, also enhances the secretion of HMW adiponectin, whereas treatment of cells with resveratrol, an activator of SIRT1, significantly reduces secretion of the complexes. Moreover, knock down of SIRT1 in 3T3-L1 adipocytes through the ectopic expression of a corresponding siRNA also increases secretion of HMW adiponectin as well as prevents the decline in PPARγ activity that normally occurs during terminal adipogenesis in 3T3-L1 cells. We next questioned whether SIRT1 controls the expression or activity of a component of the machinery participating in the formation of HMW adiponectin in the endoplasmic reticulum of the adipocyte. The results demonstrate an extensive induction of the ER oxidoreductase, Ero1-Lα, during the differentiation of 3T3-L1 preadipocytes into adipocytes that coincides with the differentiation-associated increase in secretion of adiponectin. Moreover, the data show that expression of Ero1 mRNA is enhanced several fold following exposure of adipocytes to the PPARγ ligand troglitazone, consistent with wit17 its gene being responsive to PPARγ activity. Additionally, suppression of SIRT1 activity through expression of siRNA also leads to a dramatic increase in Ero1-Lα production, along with enhanced secretion of HMW adiponectin. A direct role for Ero1-Lα in controlling adiponectin secretion was demonstrated by suppressing its production through the transient expression of Ero1-Lα RNAi during the terminal phase of adipogenesis in 3T3-L1 preadipocytes. Finally, we demonstrate that ectopic expression of Ero1-Lα leads to an extensive increase in secretion of HMW adiponectin in 3T3 adipocytes, which overcomes the negative effects of activation of SIRT1 on adiponectin secretion.

The most likely mechanism by which SIRT1 regulates adiponectin secretion is through its ability to regulate PPARγ activity and, in so doing, also regulate expression of the PPARγ-responsive gene Ero1-Lα. It is interesting that not all PPARγ-responsive genes are influenced to the same extent as Ero1-Lα by SIRT1. Specifically, knock down of SIRT1 in 3T3-L1 adipocytes leads to a dramatic increase in Ero1-Lα production, but results in no detectable change in adiponectin or C/EBPα synthesis and only a modest increase in expression of fatty acid binding protein 4 (aP2). It is important to mention that recent studies by Qiao and Shao, 2006 have suggested that SIRT1 positively regulates adiponectin gene expression, which would appear to contradict with results presented here (28). The data in those studies, however, show that suppression of SIRT1 by siRNA technology leads to a decrease in cellular amounts of adiponectin based on western blot analysis of total cell proteins. Those observations are consistent with data presented here in FIG. 4 since suppression of SIRT1 enhances adiponectin secretion with a corresponding decrease in intracellular adiponectin. Since Qiao and Shao did not measure adiponectin secretion or mRNA levels it is not possible for them to draw any strong conclusions about the precise mechanism by which SIRT1 controls adiponectin production.

Taken together, the data suggest that SIRT1 might be controlling the activity of factors, aside from PPARγ, that control Ero1-Lα gene expression, but have no role to play in control of other adipogenic genes. This notion is supported by the fact that Ero1-Lα, is also produced in a non-adipogenic manner since it is expressed in preadipocytes as well as other cell types (24). The other adipogenic genes such as adiponectin and aP2 are adipocyte-spec consequently, their expression in the adipocyte depends primarily on PPARγ active y. 3 is important to point out, however, that the enhancement of Ero1-Lα expression during adipogenesis does involve PPARγ, in this regard, it is conceivable that SIRT1 regulates PPARγ on the Ero1-Lα gene promoter differently from PPARγ associated with the other adipogenic gene promoters. In fact, our data show that mutation of F372 within helix 7 of the ligand-binding domain of PPARγ to an alanine generates a nuclear receptor that can respond to troglitazone by inducing adiponectin and aP2 expression, but is incapable of enhancing production of Ero1-Lα (FIG. 8A, lane F). Wild type PPARγ, however, is capable of inducing all three genes in response to the ligand (FIG. 8A, lane WT.). These observations suggest that PPARγ might be capable of regulating multiple functions of the adipocyte by regulating defined groups of genes in response to a select set of metabolic effectors. For instance, PPARγ might respond to one set of effectors to enhance target gene expression, whilst responding to another set to reduce expression of a different target gene. In fact, studies by Lazar and coworkers (6, 14, 20) have shown that PPARγ represses the expression of the glycerol kinase and oxidized LDL receptor genes in mature adipocytes whilst activating expression of other adipogenic genes such as aP2. We suggest that Ero 1-Lα also belongs to a similar group of adipocyte genes that are actively repressed by PPARγ in adipocytes by mechanisms that involve SIRT1.

The data in FIG. 8C support the notion that Ero1-Lα is playing a critical role in controlling the secretion of HMW adiponectin from adipocytes. The most likely mechanism involves Ero1-Lα functioning as an ER membrane-associated oxidoreductase by utilizing the oxidizing power of oxygen to generate disulfide bonds in itself, which it then transfers to PDI and the resulting oxidized PDI is then able to transfer its disulfide bonds to adiponectin. It is also possible that Ero1-Lα participates in the release of adiponectin from the adipocyte by disrupting its retention in the endoplasmic reticulum by a process referred to as thiol-retention of secretory proteins (1, 3, 15, 23). Studies have shown that another ER-resident enzyme, ERp44, retains unassembled immunoglobulin-μ chains in the cell through direct disulfide bond linkage (1). Disruption of this linkage through treatment of cells with reducing agents causes release of the immunoglobulin chains into media. Additionally, investigations have also shown that ERp44 can interact with Ero fi-Lot through a similar thiol retention process (23). It is conceivable, therefore, that adiponectin might also be retained in the adipocyte through disulfide-bond formation with ERp44 or other related proteins; and that overexpression of Ero1-Lα disrupts this interaction, thereby enhancing the release of HMW adiponectin into the medium. In fact, our preliminary data support the notion of thiol-retention of adiponectin since treatment of 3T3-L1 adipocytes with β-mercaptoethanol significantly enhances the secretion of adiponectin (data not shown).

It is also important to consider other processes that might result in reduced adiponectin secretion in addition to the apparent decrease in Ero1-Lα expression. In this regard, it is possible that mature adipocytes suffer from both oxidative as well ER stress as a direct result of the extensive secretion of disulfide-linked proteins. The secretory process can generate reactive oxygen species (ROS) as a byproduct of the oxidoreductases in producing disulfide bonds. Additionally, it is very likely that unfolded proteins will also accumulate in the ER due to inefficient folding being a byproduct of the active secretion process. Such stress responses could possibly feed back on the secretion of adipocyte proteins to allow for degradation of any unfolded proteins via ERAD and glutathione-mediated neutralization of ROS (5, 7, 34). It will be important in future investigations to determine whether there is a direct link between ER stress and adiponectin secretion. In this regard, it will also be interesting to determine whether Ero1-Lβ, which is involved in the unfolded protein response, plays a role in regulating the function of adipocytes.

In conclusion, these investigations have uncovered a novel mechanism by which adipocytes can regulate secretion of adiponectin in response to changes in nutrient status. Of interest is the demonstration that SIRT1 appears to play an important role in mediating the response of adipocytes to perturbations in overall metabolism. SIRT1 has also been implicated in processes contributing to diminishment of bodily functions associated with aging (2, 33); consequently, it will he important to determine whether there is any role for the SIRT1-associated regulation of adiponectin in the development of insulin resistance and type 2 diabetes in the elderly. Since it is well accepted that adiponectin acts to sensitize the organism to insulin, these observations should provide information that lead to development of therapeutics to combat insulin resistance and type 2 diabetes,

### REFERENCES

1. Anelli, T., M. Alessio, A. Bachi, L. Bergamelli, C. Bertoli, S. Camerini, A. Mezghrani, E. Ruffato, T. Simmer, and R. Sita. 2003. Thiol-mediated protein retention in the endoplasmic reticulum: the role of ERp44. Embo J 22:5015-22.
2 Bordine, L., and L. Guarente. 2005. Calorie restriction, SIRT1 and metabolism: understanding longevity. Nat Rev Mol Cell Biol 6:298-305.
3. Brewer, J. W., and R. B. Corley. 1996. Quality control in protein biogenesis: thiol-mediated retention monitors the redox state of proteins in the endoplasmic reticulum. J Cell Sci 109 (Pt 9):238392.
4. Cabibbo, A., M. Pagani, M. Fabbri, M. Rocchi, M. R. Farmery, N. J. Bulleid, and R. Sitia. 2000. ERO1-L, a human protein that favors disulfide bond formation in the endoplasmic reticulum. J Biol Chem 275:4827-33.
5. Chakravarthi, S., C. E. Jessop, and N. J. Bulleid. 2006. The role of glutathione in disulphide bond formation and endoplasmic-reticulum-generated oxidative stress. EMBO Rep 7:271-5.
6. Chui, P. C., H. P. Guan, M. Lehrke, and M. A. Lazar. 2005. PPARgamma regulates adipocyte cholesterol metabolism via oxidized LDL receptor 1. J Clin Invest 115:2244-56.
7. Denic, V., E. M. Quart, and J. S. Weissman. 2006. A luminal surveillance complex that selects misfolded glycoproteins for ER-associated degradation. Cell 126:349-59.
8. Dias-Gunasekara, S., and A. M. Benham. 2005. Defining the protein-protein interactions of the mammalian endoplasmic reticulum oxidoreductases (EROs). Biochem Soc Trans 33:1382-4.
9. Dias-Gunasekara, S., J. Gubbens, M. van Lith, C. Dunne, J. A. Williams, R. Kataky, D. Scoones, A. Lapthorn, N. J. Bulleid, and A. M. Benham. 2005. Tissue-specific expression and dimerization of the endoplasmic reticulum oxidoreductase Ero1beta. J Biol Chem 280:33066-75.
10. Ellgaard, L., and L. W. Ruddock, 2005. The human protein disulphide isomerase family: substrate interactions and functional properties. EMBO Rep 6:28-32.
11. Fassio, A., and R. Sitia. 2002. Formation, isomerisation and reduction of disulphide bonds during protein quality control in the endoplasmic reticulum. Histochem Cell Biol 117:151-7.
12. Frand, A. R., and C. A. Kaiser. 1998. The ERO1 gene of yeast is required for oxidation of protein dithiols in the endoplasmic reticulum. Mol Cell 1:161-70,
13. Fraud, A. R., and C. A. Kaiser. 1999. Ero1p oxidizes protein disulfide isomerase in a pathway for disulfide bond formation in the endoplasmic reticulum. Mol Cell 4:469-77.
14. Guan, H. P., Y. Li, M. V. Jensen, C. B. Newgard, C. M. Steppan, and M. A. Lazar. 2002, A futile metabolic: cycle activated in adipocytes by antidiabetic agents. Nat Med 8:1122-8.
15. Isidoro, C., C. Maggioni, M. Demoz, A. Pizzagalli, A. M. Fra, and R. Sitia. 1996. Exposed think confer localization in the endoplasmic reticulum by retention rather than retrieval, Biol Chem 271:26138-42,
16. Iwaki, M. M. Matsuda, N. Maeda, T. Funahashi, Y. Matsuzawa, M. Makishima, and I. Shimomura. 2003. Induction of adiponectin, a fat-derived antidiabetic and antiatherogenic factor, by nuclear receptors. Diabetes 52:1655-63,
17. Kadowaki, T., and T. Yamauchi. 2005. Adiponectin and adiponectin receptors. Endocr Rev 26:439-51.
18. Kadowaki, T., T. Yamauchi, N. Kubota, K. Hara, K. Ueki, and K. Tobe, 2006. Adiponectin and adiponectin receptors in insulin resistance, diabetes, and the metabolic syndrome. J Clin Invest 116:1784-92.
19. Kershaw, E. E., and J. S. Flier. 2004. Adipose tissue as an endocrine organ. J Clin Endocrinol Metab 89:2548-56.
20. Lehrke, M., and M. A. Lazar. 2005. The many faces of PPARgamma. Cell 123:993-9.
21. Liu, J., H. Wang, V. Zuo, and S. R. Farmer. 2006. Functional interaction between peroxisome proliferator-activated receptor gamma and beta-catenin. Mol Cell Biol 26:5827-37.
22. Must, A., and S. E. Anderson. 2003. Effects of obesity on morbidity in children and adolescents. Nutr Clin Care 6:4-12.
23. Otsu, M., G. Bertoli, C. Fagioli, E. Guerini-Rocco, S. Nerini-Molteni, E. Ruffato, and R. Sitia. 2006. Dynamic retention of Ero1alpha and Ero1 beta in the endoplasmic reticulum by interactions with PDI and ERp44. Antioxid Redox Signal 8:274-82.
24. Pagani, M., M. Fabbri, C. Benedetti, A. Fassio, S. Pilati, N. J. Bulleid, A. Cabibbo, and R. Sitia. 2000. Endoplasmic reticulum oxidoreductin 1-lbeta (ERO1-Lbeta), a human gene induced in the course of the unfolded protein response. J Biol Chem 275:23685-92.
25. Pajvani, U. B., M. Hawkins, T. P. Combs, M. W. Rajala, T. Doebber, J. P. Berger, J. A. Wagner, M. Wu, A. Knopps, A. H. Xiang, K. M. Utzschneider S. E. Kahn, J. M. Olefsky, T. A. Buchanan, and P. E. Scherer. 2004. Complex distribution, not absolute amount of adiponectin, correlates with thiazolidinedione-mediated improvement in insulin sensitivity. J Biol Chem 279:12152-62.
26. Picard, F., M. Kurtev, N. Chung, A. Topark-Ngarm, T. Senawong, R. Machado De Oliveira, M. Leid, M. W. McBurney, and L. Guarente. 2004. Sirt1 promotes fat mobilization in white adipocytes by repressing PPAR-gamma. Nature 429:771-6.
27. Pollard, M. G., K. J. Travers, and J. S. Weissman° 1998. Ero1p: a novel and ubiquitous protein with an essential role in oxidative protein folding in the endoplasmic reticulum, Mol dell 1:17-82.
28. Qiao, L., and J. Shao. 2006. SIRT1 regulates adiponectin gene expression through Foxo1-C/enhancer-binding protein alpha transcriptional complex. J Biol Chem 281:39915-24,
29. Rasouli, N., A. Yao-Borengasser, L. M. Miles, S. C. Elbein, and P. A. Kern.

2006. Increased plasma adiponectin in response to pioglitazone does not result from increased gene expression. Am J Physiol Endocrinol Metab 290:E42-E46.
30. Rodgers, J. T., C. Lerin, W. Haas, S. P. Gygi., B. M. Spiegelman, and P. Puigserver. 2005. Nutrient control of glucose homeostasis through a complex of PGC-1alpha and SIRT1. Nature 434:113-8.
Schadinger, S. E., N. L. Bucher, B. M. Schreiber., and S. R. Farmer. 2005. PPARgamma2 regulates lipogenesis and lipid accumulation in steatotic hepatocytes. Am J Physiol Endocrinol Metab 288:E1195-205.
32. Sevier, C. S., and C. A. Kaiser. 2002. Formation and transfer of disulphide bonds in living cells. Nat Rev Mol Cell Biol 3:836-47.
33. Sinclair, D. A., and L. Guarente. 2006. Unlocking the secrets of longevity genes. Sci Am 294:48-51, 54-7.
34. Sitia, R., and I. Braakman. 2003. Quality control in the endoplasmic reticulum protein factory. Nature 426:891-4.
35. Trujillo, M. E., and P. E. Scherer. 2005. Adiponectin-journey from an adipocyte secretory protein to biomarker of the metabolic syndrome. J Intern Med 257:167-75.
36. Tu, B. P., and J. S. Weissman. 2004. Oxidative protein folding in eukaryotes: mechanisms and consequences. J Cell Biol 164:341-6.
37. Wald, H., T. Yamauchi, J. Kamon, Y. Ito, S. Uchida, S. Kita, K. Hara, Y. Hada, F. Vasseur, P. Froguel, S. Kimura, R. Nagai, and T. Kadowaki. 2003. Impaired multimerization of human adiponectin mutants associated with diabetes. Molecular structure and multimer formation of adiponectin. J Biol Chem 278:40352-63.
38. Wilson-Fritch, L., A. Burkart, G. Bell, K. Mendelson, J. Leszyk, S. Nicoloro, M. Czech, and S. Corvera. 2003. Mitochondrial biogenesis and remodeling during adipogenesis and in response to the insulin sensitizer rosiglitazone. Mol Cell Biol 23:1085-94.
39. Yamauchi, T., J. Kamon, H. Waki, V. Terauchi, N. Kubota, K. Hara, Y. Mori, T. Ide, K. Murakami, N. Tsuboyama-Kasaoka, O. Ezaki, V. Akanuma, O. Gavrilova, C. Vinson, M. L. Reitman, H. Kagechika, K. Shudo, M. Yoda, V. Nakano, K. Tobe, R. Nagai, S. Kimura, M. Tomita, P. Froguel, and T. Kadowaki. 2001. The fat-derived hormone adiponectin reverses insulin resistance associated with both lipoatrophy and obesity. Nat Med 7:941-6.

### Example 2

Effectors e.g., activators and inhibitors) of the NAD+-dependent protein deacetylase SIRT1 can be used as therapeutics for treatment of diseases angiogenesis as a principal component of the disorder, including ischemias and cancers. The use of these compounds is based on results from recent investigations showing that inhibition of SIRT1 activity in cells leads to an increase in expression of genes that are also induced under hypoxic conditions. In fact, many of these genes have been shown to he direct targets of the hypoxia induced transcription factor, HIF1alpha. A specific gene regulated by suppression of SIRT1 activity is that coding for the angiogenesis factor vegfalpha, and, in fact, it is well known that hypoxia induces angiogenesis in many tissues as a means to compensate for the lack of oxygen. Consequently, drugs that affect SIRT1 activity can be used to regulate expression of hypoxic genes including those controlling blood vessel formation. Therapeutic angiogenesis can be used in different diseases. In the case of cancer, it can be preferable to block angiogenesis so as to starve the cancer of its blood supply, in that case activators of SIRT1 would be used to inhibit HIF1 alpha activity and vegfalpha expression. In the case of ischemias, inhibitors of SIRT1 would be useful to enhance HIF1 alpha activity and induce production of angiogenic factors.

Therapeutic angiogenesis is a means of treating patients with various ischemic and peripheral vascular diseases. Conversely, blocking angiogenesis is a strategy to treat various cancers. There are several ongoing clinical trials in which pro-angiogenic factors or inhibitors of angiogenesis are given to patients with either ischemias or cancers. In the case of therapeutic angiogenesis, a recent report by Kelly (Gene Therapy, 14:781-789, 2007) stated that clinical trials using recombinant protein or gene therapy of single angiogenic factors have yielded only modest success. An alternative approach under consideration is therapeutic expression of transgenes that enhance expression of more than one proangiogenic factor. An agent under consideration is hypoxia-induced transcription factor, HIF1alpha, which regulates expression of several proangiogenic factors and is responsible for enhancing angiogenesis in hypoxic tissues. As described herein, small molecules that modulate SIRT1 activity can be used to regulate HIF1alpha activity and therefore affect production of factors controlling angiogenesis. This is based on data showing that suppression of SIRT1 significantly enhances expression of genes that are regulated by HIF1alpha. Additionally, by targeting SIRT1, drugs that both activate and inhibit SIRT1 activity could be used to affect vascular formation in a variety of different diseases including cancer in which the therapy would involve inhibiting angiogenesis as well as ischemias to enhance blood vessel formation (see Lutton et al, Nature Medicine, 8:831-840, 2002),

Because SIRT1 regulates expression of genes induced in response to hypoxia, diseases that are affected by hypoxia are potentially treated by drugs that modulate SIRT1 activity including inflammation and neural vascular diseases.

The list of genes that are activated by HIF1alpha that are also affected by SIRT1 are listed in Table 1b of Example 3.

### Example 3

Abstract

Peroxisome proliferators-activated receptor γ (PPARγ) activity is regulated through association with ligands that include the thiazolidinedione class of anti-diabetic drugs as well as derivatives of polyunsaturated fatty acids. Induction of PPARγ target gene expression involves ligand-dependent reconfiguration of the ligand-binding domain (LBD) followed by recruitment of specific transcriptional coactivators. In this study, we have identified an amino acid (F372) within helix 7 of the LBD that is required for the response of PPARγ to endogenous ligands. Additionally, the data show that this amino acid is also required for expression of a novel subset of adipocyte genes (Group 2) including FGF21 and that the FGF21 gene is a direct target of PPARγ. Expression of the Group 2 genes is selectively repressed by the NAD-dependent deacetylase SIRT1 in mature 3T3-L1 adipocytes since knockdown of SIRT1 through the constitutive expression of a corresponding RNAi enhances their expression without affecting expression of classic adipogenic genes such as adiponectin and FABP4/aP2. It appears that many of the Group 2 genes repressed by SIRT1 in mature adipocytes correspond to the same set of genes that are selectively activated by treatment of fat cells with the PPARγ ligand, troglitazone. These data support a role for helix 7 of the LBD of PPARγ in regulating adipocyte function and suggest that inhibition of SIRT1 in adipocytes induces the same insulin-sensitizing action as PPARγ ligands.

Introduction

Peroxisome proliferators-activated receptor γ (PPARγ) is a nuclear receptor expressed in many tissues but most abundantly produced in adipose tissue where it acts as the master regulator of adipogenesis as well as a regulator of the multiple functions of mature adipocytes (6-8, 21, 32, 37). The transcriptional activity of PPARγ is regulated in part by association with lipophilic ligands that include derivatives of polyunsaturated fatty acids such as eicosinoids as well as the thiazolidinedione class of synthetic insulin sensitizers (19, 20). PPARγ consists primarily of three regulatory domains comprised of a ligand-independent transactivation domain at the N-terminus, a central DNA-binding domain and a C-terminal ligand-binding domain that facilitates ligand-dependent transactivation and heterodimerization with the retinoic acid X receptor (RXR) (18). Heterodimers of PPARγ and RXR bind to DNA consensus sites within the promoters/enhancers of target genes, which consist of direct repeats of the nuclear receptor half site spaced by a single base pair (DR-1). Activation of transcription at these target genes involves a complex process in which the docked PPARγ/RXR heterodimers, following association with ligands, recruits a series of coactivators including the p160/SRC family members that initiate formation of the RNA polymerase II/transcriptional complex involving components of the Mediator complex (11, 24, 29).

Our understanding of the mechanisms by which PPARγ activates transcription has been derived from studies employing synthetic ligands such as thiazolidinediones (TZDs). It is generally accepted that in the unliganded state PPARγ associates with the corepressors NCoR or SMRT to repress target gene expression. Entry of the thiazolidinedione into the large ligand-binding pocket stabilizes helix 12 of the transactivation domain 2 (AF2), which dislodges the corepressors and forms a binding site for the p160 family of coactivators that facilitates the pharmacological activation of PPARγ target gene expression (24, 27). Recent studies investigating the role of PPARγ in regulating inflammatory genes in macrophages presented an additional model by which thiazolidinediones might organize the recruitment of various nuclear coregulators. In this model, TZDs induce the SUMOylation of PPARγ on K365 within helix 7 of the ligand-binding domain, which targets PPARγ to NCoR/HDAC3 complexes on inflammatory gene promoters (26). These observations suggest that helix 7, in addition to helix 12, might participate in mechanisms by which ligands regulate association of PPARγ with specific coactivators or corepressors. In support of this, our recent studies have identified helix 7 as a component of the functional interaction between β-catenin, the coactivator of the canonical Wilt signaling pathway, and PPARγ (22). Since the endogenous ligand for PPARγ has not as yet been identified, the physiological mechanisms by which PPARγ regulates target gene expression in various cell types are not known.

Differentiation o preadipocytes into adipocytes depends on stimulation of PPARγ activity that is facilitated by a C/EBPβ-associated induction of PPARγ2 gene expression as well as production of endogenous ligands (8). As mentioned above, the physiological ligand for PPARγ has not been identified, but recent studies suggest that signaling pathways involving cAMP, C/EBPβ and xanthine oxidoreductase activate a transient increase in ligand production during the initial 2-4 days of adipogenesis in 3T3-L1 preadipocytes (4, 14, 23, 35). ‘The level/activity of these ligands subsides dramatically during terminal differentiation to the extent that mature adipocytes express low levels of activity. Despite this apparent decrease in endogenous ligand activity, PPARγ is capable of maintaining expression of most of its target genes in mature adipocytes. In this regard, it is interesting that some target genes are expressed at low levels in adipocytes, but are responsive to activation of PPARγ by TZDs. Specifically, genes coding for glycerol kinase (GyK) and the oxidized LDL receptor (OLR-1) are PPARγ target genes that are normally expressed at low abundance in white adipose tissue and mature adipocytes in culture. Exposure of 3T3-L1 adipocytes to TZDs induces transcription of mRNAs for GyK and OLR-1 (5, 13). Additional studies by Lazar and coworkers (12) have shown that PPARγ is bound to PPAR response elements (PPREs) in the promoter of the transcriptionally inactive GyK gene in mature adipocytes in addition to being bound to the enhancer of the transcriptionally active aP2 gene. The data suggest that endogenous ligands are unable to dislodge corepressors from PPARγ on the GyK gene, but do facilitate this process along with recruitment of p160 coactivators to PPARγ on the aP2 gene. Moreover, it appears that exposure of mature adipocytes to TZDs can activate GyK expression by regulating the switch in corepressor/coactivator recruitment to the PPARγ bound to the corresponding promoter.

In the present study, we investigated mechanisms by which PPARγ might expression of select genes in response to different effectors. The data show that PPARγ regulates expression of at least two programs of gene expression during adipogenesis in 3T3-L1 preadipocytes: The one program (Group 1) consists of classic adipogenic genes including FABP4/aP2, adiponectin and perilipin and, following its induction; this program continues to be expressed throughout terminal adipogenesis and in mature adipocytes. The other program (Group 2) consists of a diverse array of genes some of which appear to be involved in glucose homeostasis and insulin action including FGF21 and the oxidoreductase Ero1-Lα. Expression of these Group 2 genes can be selectively activated in mature adipocytes by synthetic PPARγ ligands or suppression of SIRT1 activity. Our studies also show that helix ‘7 within the ligand-binding domain plays a critical role in the response of PPARγ to endogenous ligands. Mutation of select amino acids within helix 7 specifically F372 renders PPARγ completely incapable of activating adipogenic gene expression in response to endogenous ligand activity. Exposure of cells expressing the mutant F-PPARγ to thiazolidinediones induces expression of the adipogenic program containing adiponectin and aP2, but is incapable of inducing the program containing FGF2 1 and Ero1-Lα.

Materials and Methods

Materials: Dexamethasone (DEX), 3-isobutyl-1-methylxanthine (MIX), insulin were purchased from Sigma (St. Louis, Mo.). Leupeptin, aprotinin, and puromycin were purchased from American Bioanalytical (Natick, Mass.), while Dulbecco's modified Eagle's medium (DMEM) were purchased from Mediatech, Inc (Herndon, Va.), calf serum and TRIzol were purchased from Invitrogen (Carlsbad, Calif.). Fetal bovine serum (FBS) was obtained from Gemini Bio-Products and troglitazone was obtained from Biomol International.

Antibodies: Monoclonal anti-PPARγ antibody and polyclonal C/EBPα were purchased from Santa Cruz Biotechnology (Santa Cruz, Calif.), polyclonal aP2 serum was kindly provided by Dr. D. Bernlohr, (University of Minnesota) while anti-perilipin antibody was kindly provided by Dr. A. Greenberg (Tufts University, Boston, Mass.) and polyclonal anti-ACRP30 (adiponectin) was obtained from Affinity BioReagents (Golden, Colo.). Anti-Ero1 polyclonal antibody was purchased from Abnova Co., (Taiwan, China).

Plasmids and Cell Lines: Replacement of phenylalanine 372 of PPARγ1 with an alanine was achieved by performing site-directed mutagenesis of the pBabe-WT.-PPARγ plasmid using QuickChange II XL kit (Stratagene) following the manufacturers instructions. pSUPER-SIRT1 siRNA plasmid was generously provided by Dr Jim Xiao of Boston University School of Medicine and consisted of the vector recently described (28). Generation of appropriate retrovirus particles was as follows: HE grown to 70% confluence in 100 mm-diameter dishes at which stage they were transfected with the DNA-FUGENE cocktail consisting of 36 μL Fugene 6, 6 μg retrovirus plasmid, 6 μg pVPack-VSV-G vector, 6 μg pVPack-GAG-POL vector and 164 μL DMEM without FBS. Twenty-four hours later, the medium was replaced with 6 mL fresh DMEM containing 10% FBS. One day after that, the culture medium containing high-titer retrovirus was harvested and filtered through a 0.45 nm pore size filter. The viral filtrate was used to infect both 3T3-L1 preadipocytes (control and SIRT1 siRNA cells) and Swiss 3T3 fibroblasts (WT.-, E-, EF-, F-, DD-PPARγ cell lines).

Cell Culture: The Swiss fibroblast cell lines expressing wild type (WT.) and mutant forms of PPARγ (E, EF, F, DD) generated as previously described (22) and murine 3T3-L1 preadipocytes were grown in Dulbecco's modified Eagle's medium (DMEM) containing 10% FBS (fibroblasts) or 10% calf serum (preadipocytes) until confluent and were then maintained in the same medium for an additional 2 days. Differentiation was induced at 2 days postconfluence (day 0) by adding fresh DMEM containing 10% FBS, 0.5 mM MIX, 1 μM DEX, 1.67 μM insulin with or without 5 μM troglitazone. The immortilized primary brown preadipocytes (gift of Dr C R Kahn, Joslin Diabetes Center, Boston, (9, 10)) were grown to confluence in differentiation medium composed of DMEM containing 10% FBS supplemented with 20 nM insulin and 1 nM 3,3′,5-triiodo-L-thyronine [T3]). After 2 days post-confluence, cells were induced to differentiate by exposure to DEX, MIX, insulin, 0.125 mM indomethacin and 10% FBS. Cells were refed every 2 days.

Microarray Gene Chips: Swiss W7,-PPAR γ and EF PPAR γ cells were differentiated in the presence or absence of troglitazone for 5 days as described above. Additionally, control and SIRT1 knockdown 3T3-L1 preadipocytes were differentiated as described above for 0, 4 and 10 days (see Tables 1A and 1B), Total RNA was isolated from all cells using Trizol Reagent (Invitrogen and microarray analysis was performed by the Microarray Resource (Boston University School of Medicine). Briefly, double-stranded cDNA was synthesized from 10 μg of RNA using SuperScript double-stranded cDNA synthesis kit (Invitrogen) and purified using a Phase-Lock Gel (PLC Heavy Brinkmann Instruments, Westbury, N.Y.). Biotin-labeled cRNA was then generated using RNA Transcript Labeling Kit (Enzo Diagnostics, Farmingdale, N.Y.) and purified using RNeasy affinity columns (Qiagen). After treatment at 94° C. for 35 min in 40 mM Tris-acetate, pH 8.1, 100 mM KOAc, 30 mM MgOAc, 15 μg of fragmented cRNA was hybridized to the Affymetrix GeneChip mouse Expression Set MOE430A2.0 array at 45° C. for 16 h and 60 rpm using controls supplied by the manufacturer (Affymetrix). Arrays were then washed and stained according to the standard Antibody Amplification for Eukaryotic Targets protocol (Affymetrix). The stained Gene Chip arrays were scanned at 488 nm using an Affymetrix Gene Chip Scanner 3000 (Affymetrix). The scanned images were then quantified and scaled using Microarray Suite 5.0 software (Affymetrix).

Oil Red O Staining: The cells were seeded in 35-mm plates, and at the specified stage of differentiation they were rinsed with PBS and fixed with 10% formalin in PBS for 15 min. After two washes in PBS, cells were stained for at least 1 h in freshly diluted Oil Red O solution (6 parts Oil Red O stock solution and 4 parts H₂O; Oil Red O stock solution is 0.5% Oil Red O in isopropyl alcohol). The stain was then removed, and cells were washed twice with water and then photographed.

Western Blot Analysis of Proteins: Equal amounts of protein extracted from the total cell layer were fractionated on 8% SDS-polyacrylamide gels and transferred to polyvinylidene difluoride membranes (PerkinElmer Life Sciences). Following transfer, the membranes were blocked with 10% nonfat dry milk in phosphate-buffered saline, 0.1% Tween 20 and probed with the antibodies corresponding to the various target proteins indicated in each figure. Horseradish peroxidase-conjugated secondary antibodies (Sigma) and an ECL substrate kit (PerkinElmer Life Sciences) were used for detection of specific proteins.

Analysis of RNA: Total cellular RNA was prepared using TRIzol reagent (InVitrogen) according to the manufacturer's instructions. As were made from equivalent amounts of total RNA by using Reverse Transcription System (Promega) as described previously (33). Primer sequences used for amplification were synthesized Integrated DNA Technologies Inc., Coralville, Iowa) to be specific for:

| | |
|---|---|
| PPARγ forward primer | GAGCATGGTGCCTTCGCTGAT |
| PPARγ reverse primer | CAACCATTGGGTCAGCTCTTG |
| C/EBPα forward primer | AAGGTGCTGGAGTTGACCAGT |
| C/EBPα reverse primer | TAGAGATCCAGCGACCCGAAA |
| PGC-1α forward primer | GTCAACAGCAAAAGCCACAA |
| PGC-1α reverse primer | TCTGGGGTCAGAGGAAGAGA |
| pex11a forward primer | CCGACTTTTCAGAGCCACTC |
| pex11a reverse primer | CGGTTAGGTTGGCTAATGT |
| ERO1 forward primer | GAAGGATCCATGGGCCGCGCCTGGG |
| ERO1 reverse primer | CGCCGTCGACGGCACATTCCAACCG |
| OLR1 forward primer | GTCATCCTCTGCCTGGTGTT |
| OLR1 reverse primer | TTCTTCCGATGCAATCCAAT |
| ELOVL3 forward primer | TCGTCTGCAAAATCGAAATG |
| ELOVL3 reverse primer | GGGAAACCATACAGGGAGGT |
| FGF21 forward primer | CTGGGGGTCTACCAAGCATA |
| FGF21 reverse primer | AAGGCTCTACCATGCTCAGG |
| mGST1 forward primer | ATGAGGTGTTGATGGCCTTT |
| mGST1 reverse primer | GGTTTCCCATAGGTGTGTGC |
| Nr1h3 (LXRα) forward | CCTGATTCTGCAACGGAGTT |
| primer | |
| Nr1h3 (LXRα) reverse | GGCTCACCAGCTTCATTAGC |
| primer | |
| Ephx2 forward primer | CCTGGCACTGCCTAGAGACT |
| Ephx2 reverse primer | GCTTTATGCACAGCGATGAA |
| Scd3 forward primer | TGCTGCAAGAAGAGATGACG |
| Scd3 reverse primer | CTCTTGTGACTCCCGTCTCC |
| adipoq forward primer | GTTGCAAGCTCTCCTGTTCC |
| adipoq reverse primer | CAGACTTGGTCTCCCACCTC |
| GyK forward primer | CCGAAGGAAATTCTGCAGTC |
| GyK reverse primer | CGCCTAGTGCAGTTGTTTCA |
| aP2 forward primer | AATGTGTGATGCCTTTGTGG |
| aP2 reverse primer | AATTTCCATCCAGGCCTCTT |
| Ndrg1 forward primer | CGAGAGCTACATGACGTGGA |
| Ndrg1 reverse primer | CTGGCAGAAGGCATGTATCC |
| Egln3 forward primer | GAGATGCCTCTGGGACACAT |
| Egln3 reverse primer | TTCTGCCCTTTCTTCAGCAT |
| Egln1 forward primer | AGCCATGGTTGCTTGTTACC |
| Egln1 reverse primer | TGTCCACGAGTCCACAGAAG |
| Cbr1 forward primer | GACCGGTGCTAACAAAGGAA |
| Cbr1 reverse primer | GCTCCTTCTTCTGGGCTTTT |
| Mrap forward primer | CTTCGTGGTGCTCCTCTTTC |
| Mrap reverse primer | GACCTGGTCCTAGGGGAGAG |
| Trib3 forward primer | GATGCCAAGTGTCCAGTCCT |
| Trib3 reverse primer | TCTCCCTTCGGTCAGACTGT |
| Ndg2 forward primer | CGGGTCTTATGGCTCAGATG |
| Ndg2 reverse primer | GAAGCCCTCACACAGGGTTA |
| Cox7a1 forward primer | GCTCTGGTCCGGTCTTTTAG |
| Cox7a1 reverse primer | CCAGCCCAAGCAGTATAAGC |
| Adipsin forward primer | TGATGTGCAGAGTGTAGTGCCTCA |
| Adipsin reverse primer | ACGTAACCACACCTTCGACTGCAT |
| Hsd11b forward primer | CAGAAATGCTCCAGGGAAAG |
| Hsd11b reverse primer | GATCTTCCTTCCTGGGTTCC |
| UCP-1 forward primer | TCTTCTCAGCCGGAGTTTCAGCTT |
| UCP-1 reverse primer | TGTTGACAAGCTTTCTGTGGTGGC |
| GAPDH forward primer | ATCACCATCTTCCAGGAGCGA |
| GAPDH reverse primer | GTTGTCATGGATGACCTTGGCC |

Plasmid Constructs and Luciferase Reporter Gene Assays: The mouse FGF21 promotor constructs −1537/+54, −1299/+54, and −553/+54 were generated by PCR using C57BL/6NCrl mouse genomic DNA and the following oilgonucleotides: −1537 forward, 5′-AAGCCTCACCTTGACACC-3′, −1299 forward, 5′-CAGGAAACAACCCAGCTC-3′; −553 forward, 5′-AGTGCAGACAAGTCCCCT-3′; +54 reverse, 5′-GGCAGCTGGAATTGTGTT-3′. The PCR-amplified fragments were cloned into Kpn1 and Bgl2 sites of the luciferase reporter plasmid pGL3, For transfection assays, the Swiss fibroblasts (control expressing a pBabe-puro empty vector) or cells expressing a WT.-PPARγ were seeded in 24-well plates in triplicate for 24 hr at which time 500 ng of the FGF21 promoter plasmids or pBabe-PPARγ plasmid plus 20 ng of Renilla luciferase plasmid were transfected into each well using FuGENE 6 (DNA: FuGENE 6=1:6). Twenty-four hours later, when appropriate, the cells were treated for 48 hours with 1 μM GW1929 and were then washed twice with phosphate-buffered saline and lysed with 100 μl of passive lysis buffer. Luciferase/Renilla assays were performed using the Dual-Luciferase Reporter Assay System kit (Promega, Madison, Wis.) and a Luminoskan Ascent luminometer (Thermo Labsystems, Franklin, Mass.). The average ratio (from three wells) of luciferase activity (relative light units) to Renilla activity was calculated. The same experiment was repeated at least three times. The final values/standard deviation was calculated based on all repeats.

Results

Our previous investigations demonstrated that four amino acids, E367, F372, D378 and D379, within helix 7 of the ligand-binding domain of PPARγ facilitate a functional interaction between PPARγ and β-catenin (22). To gain insight into the potential involvement of helix 7 in regulating the transcriptional activity of PPARγ during adipogenesis, we expressed a series of mutant PPARγ proteins in Swiss 3T3 fibroblasts in which E367, F372, D378 or D379 were modified to alanine and assessed their ability to induce adipogenic gene expression. First, we observed that ectopic expression of the WT.-PPARγ was capable of inducing the conversion of these fibroblasts into adipocytes simply by exposure to DEX, MIX and insulin without the need for an exogeneous ligand such as troglitazone (data not shown). In the experiments, Swiss cells expressing various forms of PPARγ (WT, E, EF, DD) were cultured until confluent, after 2 days, they were exposed to DEX, MIX, insulin, with or without 5 μM troglitazone. A, Day 5 cells were fixed, stained with Oil Red O and photographed.

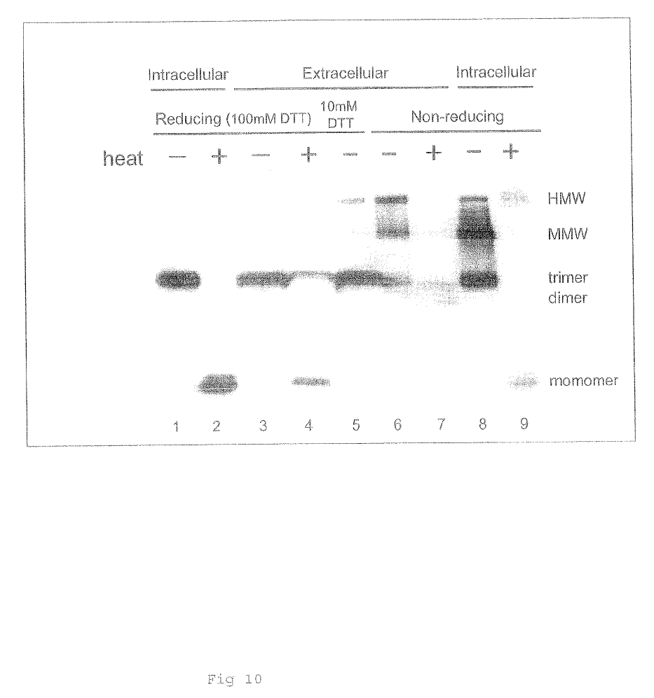

These data suggest that Swiss fibroblasts produce endogenous ligands that can activate the ectopic PPARγ following exposure to the normal cocktail of adipogenic inducers. In fact, exposure to troglitazone appears to have no additional effect on the morphological features of these Swiss adipocytes (data not shown). Additionally, the western blot shown in FIG. 11, lanes 1 and 5 shows abundant expression of the adipogenic proteins, GERM., perilipin and aP2, and a low level of β-catenin production in the Swiss-WT-PPARγ cells induced to differentiate in the presence or absence of troglitazone. The data also show that the relative abundance of transcriptionally active WT-PPARγ is very low due to its rapid turnover. The mutant PPARγ corresponding to E367A (E-PPARγ) retained the ability to induce adipogenesis in the presence or absence of troglitazone (data not shown), which included degradation of β-catenin (FIG. 11). It is interesting, however, that this alteration appears to stabilize PPARγ in the absence of ligand, while exposure to troglitazone results in a significant decrease in its abundance (FIG. 11, compare lanes 2 and 6). The most interesting data came from analyzing expression of the mutant PPARγ corresponding to E367A and F372A (EF-PPARγ). FIG. 11 demonstrates that mutation of F372 to alanine, in addition to E367A, completely destroys the ability of PPARγ to respond to an endogenous ligand since Swiss cells expressing EF-PPARγ remain as fibroblasts (data not shown) and do not express adipogenic genes or down regulate β-catenin (FIG. 11). Additionally, this mutant PPARγ appears to be quite stable. More importantly, exposure of the Swiss-EF-PPARγ cells to troglitazone induces their conversion into adipocytic cells) and expression of C/EBPα, perilipin and aP2 (FIG. 11). These data are consistent with the notion that F372 and E367 within helix 7 participate in the response of PPARγ to endogenous ligands, whereas responses to exogenous synthetic ligands such as troglitazone are less dependent on these amino acids. Mutation of both D378 and D379 to alanine completely destroys the ability of PPARγ to respond to both endogenous and exogenous ligands, since the corresponding mutant PPARγ is incapable of inducing either morphological conversion or adipogenic gene expression (data not shown and FIG. 11, lanes 4 and 8).

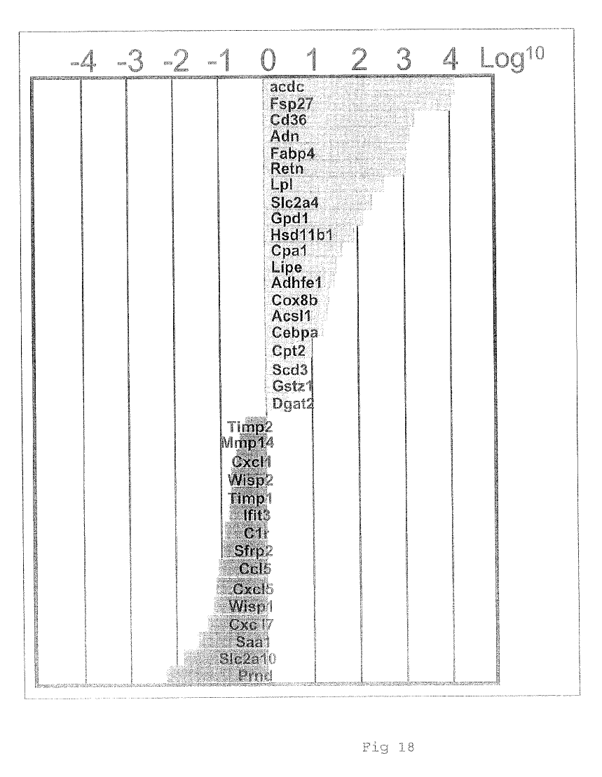

The data presented in FIG. 11 suggested to us that analysis of mRNA expression it Swiss WT-PPARγ versus Swiss-EF-PPARγ cells might permit the identification of PPARγ target gene programs responding to endogenous versus exogenous ligands. Consequently, total RNA was harvested from Swiss fibroblasts expressing either WT- or EF-PPARγ proteins 5 days following exposure to the adipogenic inducers in the presence or absence of troglitazone and subjected to oligonucleotide microarray analysis employing affymetrix chips. Total RNA of WT-PPARγ and EF-PPARγ cells at day 5 (with or without troglitazone) were isolated using Trizol Reagent (Invitrogen) and microarray analysis was performed as described in materials and methods. The data reveal that the abundance of 1767 genes of the ˜22,690 represented on the array differed at least 2 fold between the highly differentiated Swiss-WT-PPARγ cells and the undifferentiated Swiss-EF-PPARγ cells (minus troglitazone). A cluster analysis of these genes that are highly expressed in WT-PPARγ cells (minus troglitazone) relative to their expression in EF-PPARγ cells (minus troglitazone) were arranged in descending order of their relative abundance (data not shown). Genes that are highly expressed in adipocytes (WT-PPARγ) compared to fibroblasts (EF-PPARγ minus troglitazone) cluster together and include those coding for adipogenic, lipogenic and mitochondrial proteins. In contrast, many genes are expressed at much lower abundance in the adipocytes (WT-PPARγ cells) compared to the fibroblasts (EF-PPARγ minus troglitazone) and include components of the Wnt signaling pathway as well as inflammatory proteins, several of which have previously been reported to be down-regulated during adipogenesis. FIG. 18 represents the relative abundance of select genes present in each of these clusters and reveals that many of the genes display significantly more than a 5-fold difference in abundance between the two cell types. In fact, some mRNAs such as adiponectin (acdc) and Fsp27 are expressed at least 10⁴ fold more abundantly in the adipocytes (WT-PPARγ) as compared to fibroblasts (EF-PPARγ minus troglitazone). Treatment of the WT-PPARγ cells with troglitazone does not significantly alter the overall pattern of gene expression, but appears to enhance the level of adipogenic gene expression whilst suppressing even further the fibroblastic mRNAs (data not shown). More importantly, the EF-PPARγ cells that are completely unresponsive to endogenous ligands (-troglitazone) arc extensively induced to express multiple adipogenic, lipogenic and mitochondrial genes following their exposure to troglitazone (data not shown). These EF-PPARγ cells also downregulate expression of the fibroblastic genes in response to troglitazone consistent with them attaining an adipocyte-like morphology (data not shown).

Identification of a Subset of PPARγ-Responsive Genes

To gain more insight into the gene programs regulated by PPARγ in response to endogenous vs exogenous ligands, a more detailed analysis of individual genes was performed as shown in Tables 1A and 1B. Tables 1A and 1B show the relative level of expression (based on signal intensity on microarray) of select genes during the differentiation of Swiss-PPARγ fibroblasts in presence (+) or absence (−) of troglitazone (WT−T, WT+T, EF-T−EF+T) and control (C) and SIRT1 knockdown (Si) 3T3-L1 preadipocytes at 0, 4 and 10 days of differentiation. T=5 μM troglitazone. (A) Classic adipogenic genes that respond to troglitazone activation in EF-PPARγ cells. (B) Genes that are not activated by troglitazone in EF-PPARγ cells and are referred to in text as Group 2 genes.

We also analyzed the profile of mRNAs expressed during the differentiation of 3T3-L1 preadipocytes for comparison with the mRNAs expressed in Swiss-PPARγ cells by performing additional affymetrix array analysis of mRNAs isolated from the preadipocytes at 0, 4 and 10 days of differentiation. Table 1 A lists a selection of classic adipogenic genes that are induced to varying extents during adipogenesis in 3T3-L1 preadipocytes (columns 5, 6 and 7), which include genes coding for proteins involved in lipid storage/metabolism (i.e., FABP4) as well as endocrine functions (i.e. adiponectin). All of these mRNAs are expressed much more abundantly in Swiss fibroblasts expressing WT-PPARγ compared to cells expressing EF-PPARγ. In fact, the difference in the level of expression of these mRNAs in EF-PPARγ cells versus WT-PPARγ cells is comparable to the difference in their expression in preadipocytes versus mature adipocytes (Table 1A, compare columns 1 and 3 with columns 5 and 7). It is also relevant to point out that expression of at least three genes, Resistin (Retn), Hsd11β1 and Orosomucoid (Orm1) is enhanced by WT-PPARγ in Swiss n response to endogenous ligands (minus troglitazone) and during adipogenesis in 3T3-L1 predipocytes. Interestingly, troglitazone significantly attenuates expression of these genes in WT-PPARγ (Table 1A, Rctn, Hsd11β1 and Orm1, compare column 2 with column 1) consistent with reports that thiazolidinediones selectively repress expression of these genes following their dramatic induction during adipogenesis in 3T3-L1 cells (2, 3, 34). Taken together, the data in Table 1A are consistent with the notion that PPARγ can induce expression of the majority of the classic adipogenic genes in Swiss fibroblasts in response to an endogenous ligand to the same extent as that occurring during normal adipogenesis in 3T3-L 1 preadipocytes. Furthermore, mutation of critical amino acids within helix 7 (EF-PPARγ) prevents PPARγ from responding to endogenous ligand activity (Table 1 A, column 3). However, exposure of EF-PPARγ to troglitazone can induce expression of most of these classic adipogenic genes to levels attained in 3T3-adipocytes (Table 1A, compare column 4 with column 7 and data not shown).

Following a more extensive analysis of the array data, it was observed that not all genes that are highly expressed in 3T3-L1 or WT-PPARγ adipocytes were induced in the EF-PPARγ cells by troglitazone. In fact, it appears that activation of the mutant EF-PPARγ by the exogenous ligand, while capable of converting these fibroblasts into adipocytic cells that contain small lipid droplets and express many of the markers of mature adipocytes (FIG. 11), is incapable of inducing the entire adipogenic program (Table 1B). More specifically, the data shown in Table 1B suggest that WT-PPARγ induces expression of a group of responsive genes (columns 1 and 2) that are significantly less responsive to stimulation of EF-PPARγ by troglitazone (column 4). This subset of PPARγ target genes (referred to here as Group 2) includes proteins that have not previously been shown to be associated with PPARγ activity such as the ER oxidoreductase Ero1-Lα, FGF21, and genes coding for components of the glycolytic pathway. Table 1B also shows that some of these genes including Mrap, KLF15, Klb (βKlotho) and Pdxp, are ed several fold during adipogenesis, but are unresponsive to troglitazone activation of EF-PPARγ. Other genes are moderately responsive to adipogenic signals in 3T3-L1 preadipocytes (i.e the glycolytic genes) but almost all of these genes are induced in response to troglitazone activation of WT-PPARγ, but not of EF-PPARγ,

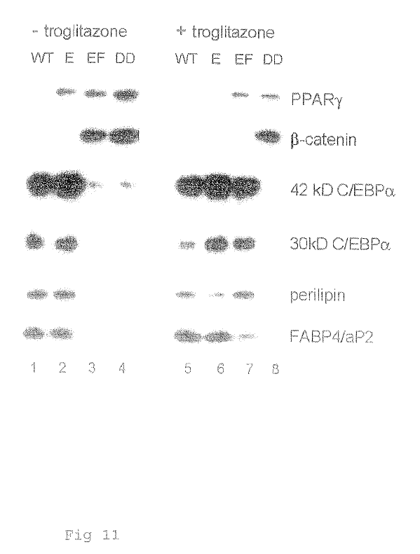

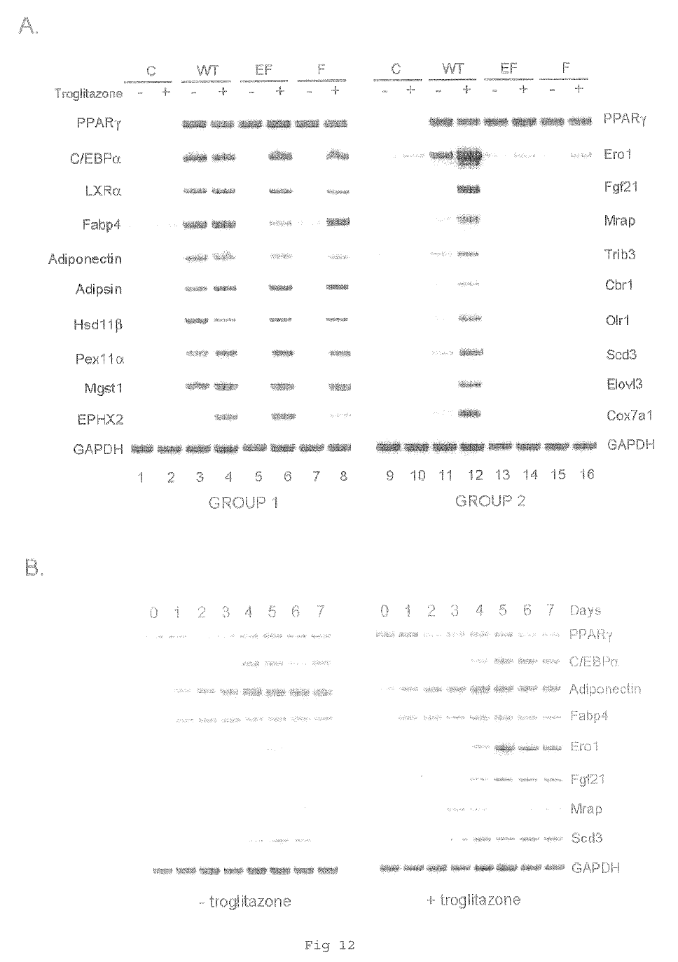

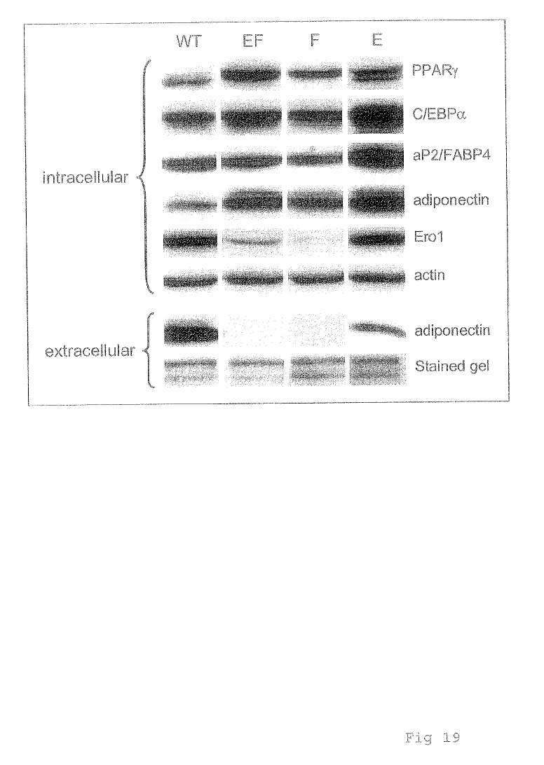

To confirm the oligonucleotide microarray data, a series of RT-PCR analyses were performed in which the relative abundance of select mRNAs expressed in the Swiss cell lines was measured. Since the data presented in FIG. 11 suggest that F372 is the amino acid that appears to be influencing the transcriptional activity of PPARγ, we generated an additional cell line corresponding to Swiss fibroblasts expressing PPARγ in which only F372 was changed to alanine (F-PPARγ cells). We also analyzed Swiss fibroblasts that do not contain an ectopic PPARγ and, therefore, are completely incapable of adipogenesis even in the presence of troglitazone (control cells). FIG. 12A shows the constitutive expression of the corresponding PPARγ mRNAs in each of the PPARγ-Swiss cell lines and the absence of any PPARγ mRNA in the control cells. The panel on the left demonstrates expression of select target genes from Table 1A that are induced in the WT-PPARγ cells exposed to endogenous (FIG. 12A, lane 3) as well as exogenous (FIG. 12A, lane 4) PPARγ ligands. Expression of most of these genes is unaffected by troglitazone with the exception of EPHX2 that appears to be enhanced even further by the exogenous ligand. This set of classic adipogenic genes, as expected, is not expressed in the EF- or F-PPARγ cells in response to the endogenous ligands (FIG. 12A, lanes 5 and 7). Furthermore, activation of these mutant cell lines (EF- and F-PPARγ) by exposure to troglitazone significantly induces expression of all of these genes (FIG. 12A, lanes 6 and 8) as shown in Table 1A. In contrast, the subset of genes (Group 2) presented in the panel on the right selected from Table 1B responds quite differently to the action of the mutant PPARγ molecules. These genes are induced to varying extents by endogenous ligands in cells expressing WT-PPARγ and are enhanced many fold by exposure to troglitazone (FIG. 12A compare lanes 11 and 12 with 9 and 10). More importantly, this subset of genes is unresponsive to activation of EF- or F-PPARγ by troglitazone as well as the endogenous ligands (FIG. 12A, compare lanes 13, 14, 15, 16 with lanes 11 and 12). We also performed western blot analysis of C/EBPα, FABP4/aP2, adiponectin (Group 1, EF-PPARγ-responsive genes) Ero1-Lα Group 2, EF-PPARγ-unresponsive gene) to confirm the RT-PCR data. FIG. 19 shows that troglitazone stimulation of all forms of PPARγ including WT-PPARγ, E-PPARγ EF-PPARγ and PPARγ lead to abundant expression of C/EBPα, FABP4/aP2 and adiponectin. In contrast, expression of Ero1-Lα is completely unresponsive to troglitazone stimulation of F-PPARγ or EF-PPARγ, but responds to WT- and E-PPARγ activity. It is interesting that analysis of the proteins in the culture media showed that adiponectin is secreted from cells expressing WT- and E-PPARγ, but is absent from the media of F-PPARγ and EF-PPARγ cells. These data suggest that some Group 2 proteins likely participate in processes responsible for secretion of adiponectin. In fact, recent studies by others and us have demonstrated a role for Ero1-Lα in regulating secretion of adiponectin from adipocytes (30, 36).

The Group 2 subset of genes can be selectively activated in response to troglitazone during the differentiation of Swiss 3T3 fibroblasts into adipocytes.

The data in FIG. 12A and Table 1B show that many of the Group 2 genes are constitutively expressed at a low level during normal adipogenesis in response to endogenous ligands, but appear to be responsive to potent exogenous ligands. To gain a greater insight into mechanisms regulating these two programs of gene expression, Swiss-WT-PPARγ cells were induced to differentiate in the absence or presence of troglitazone and expression of select genes was analyzed each day using RT-PCR technology. FIG. 12B demonstrates the constant and abundant expression of the WT-PPARγ throughout 7 days of differentiation, which resulted in a robust and sustained induction of the Group 1 genes, such as adiponectin and C/EBPα, in response to endogenous (−troglitazone) as well as exogenous ligands (+troglitazone). Interestingly, the Group 2 genes, including Ero1-Lα, Scd3 and FGF21, are transiently expressed at a very low level during the initial 2-4 days of adipogenesis and are then down-regulated as differentiation proceeds in the absence of troglitazone. Differentiation of these WT-PPARγ cells in the presence of troglitazone has a minimal effect on expression of adiponectin and C/EBPα mRNAs, but enhances as well as maintains expression of Ero1-Lα, Scd3 and FGF21 throughout the 7-day culture period.

Select Group 2 Genes are Transiently Expressed During the Differentiation of Brown and White Preadipocytes

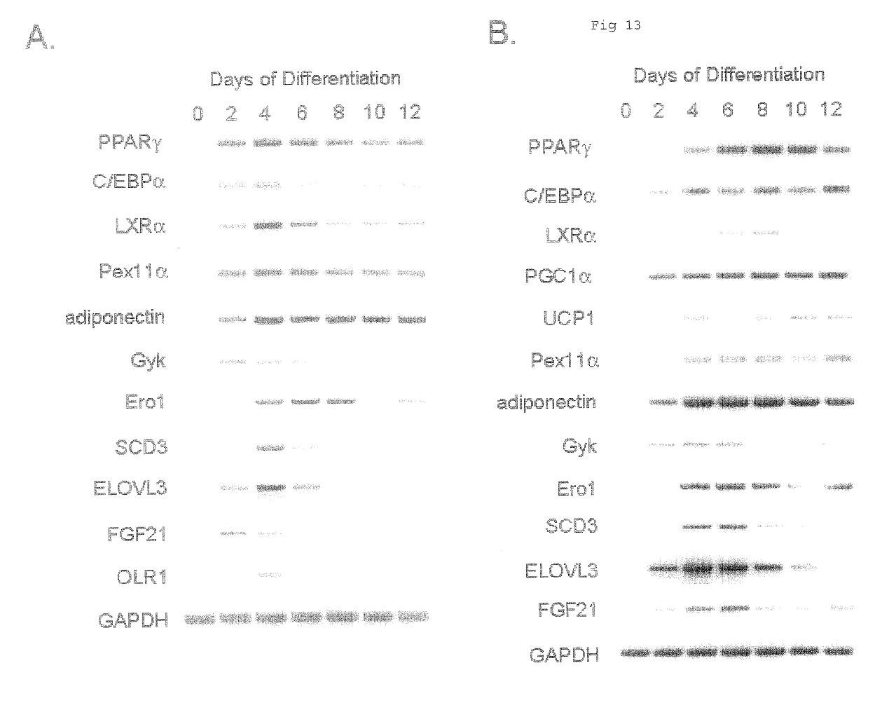

The data presented in FIGS. 12A and 2B were derived from non-adipogenic fibroblasts forced to differentiate into adipocytes by over-expression of PPARγ. We considered it important, therefore, to determine whether this interesting pattern of PPARγ target gene expression occurs in preadipocytes undergoing differentiation into brown as well as white adipocytes in response to activation of endogenous adipogenic transcription factors. To this end, we analyzed expression of genes during the differentiation of 3T3-L1 white preadipocytes and immortalized primary brown preadipocytes. FIG. 13 shows the expected induction of PPARγ, C/EBPα, LXRα and adiponectin mRNAs at 2 days following exposure of the preadipocytes to DEX, MIX, insulin and 10% FBS. Furthermore, expression of these adipogenic genes remains at a high level throughout differentiation of both brown and white preadipocytes. To confirm that the immortalized primary brown preadipocytes underwent differentiation into brown adipocytes, we also analyzed expression of PGC-1α and UCP-1, and the data show expression of these mRNAs was initiated at 2 days and was maintained throughout brown adipogenesis (FIG. 13B). In contrast, the Group 2 genes that respond poorly to expression of F-PPARγ in the Swiss cells are induced in response to activation of endogenous PPARγ in both the brown as well as the white preadipocytes, however, the level of expression of the corresponding mRNAs drops significantly during terminal adipogenesis as observed in the Swiss-WT-PPARγ cells differentiated in the absence of troglitazone (FIG. 12B).

Differential Response of Group 1 and Group 2 Genes to PPARγ Agonists and Antagonists

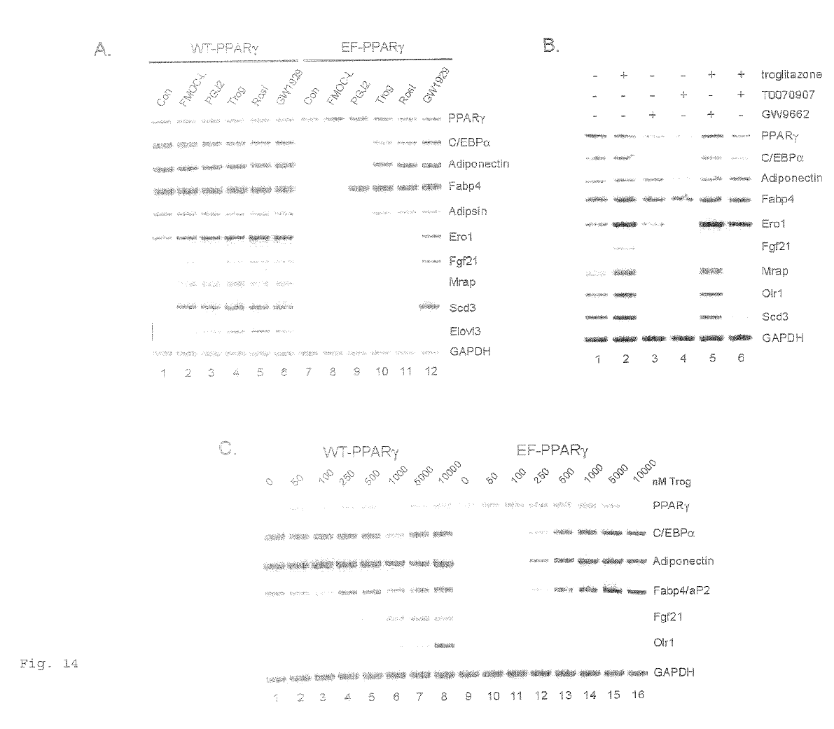

The fact that mutations within helix 7 rendered PPARγ unresponsive to endogenous ligands and responsive to troglitazone at least for the Group 1 genes, encouraged us to determine the effect of other ligands that have previously been shown to possess a range of activities. Consequently, WT-PPARγ and EF-PPARγ cells were induced to differentiate in the presence absence of 15δ-PGJ2, FMOC-leu, troglitazone, rosiglitazone or GW1929 for 5 days and expression of select genes was analyzed by RT-PCR. FIG. 14A shows that all of the exogenous ligands have little to no additional effect on the expression of select Group 1 genes in WT-PPARγ cells since their level of expression is already at a maximum due presumably to the stimulation of the ectopic PPARγ, by endogenous ligands (compare lanes 2-6 with lane). In contrast, expression of the Group 2 genes is enhanced to varying extents by exposure of the WT-PPARγ cells to the exogenous ligands. In the case of the EF-PPARγ cells, exposure to the different ligands resulted in a significantly more varied response than that observed in the WT-PPARγ cells. Specifically, FMOC-leu was incapable of stimulating expression of any of the selected Group 1 or Group 2 genes, and 15δ-PGJ2 only activated FABP4 expression. The thiazolidinediones, troglitazone and rosiglitazone, induced expression of select Group 1 genes including C/EBPα, adiponectin and FABP4, but had a negligible effect on expression of the Group 2 genes such as Ero1-Lα and Mrap. Interestingly, GW1929, an extremely potent, synthetic PPARγ ligand in which N-tyrosine moieties have been substituted for the thiazolidinedione head group, is capable of inducing expression of the Group 2 genes as well as Group 1 genes in the EF-PPARγ cells. These data clearly show a differential response of the two groups of genes to different ligands; we questioned, therefore, whether the genes also show a similar differential response to a PPARγ antagonist. To address this question, WT-PPARγ cells were induced to differentiate in the presence or absence of T0070907 or GW9662 (two PPARγ antagonists) with or without troglitazone and corresponding cellular RNAs were analyzed by RT-PCR. FIG. 14B demonstrates that T0070907 and GW9662 moderately attenuate the ability of WT-PPARγ to induce expression of C/EBPα and adiponectin in response to endogenous ligands. The presence of troglitazone overcomes the inhibitory effect of the antagonists (FIG. 14B, compare lanes 5 and 6 with lanes 3 and 4). In contrast, the antagonists almost completely block expression of the Group 2 genes including Ero1-Lα, Mrap, Elovl3, Egln1, SCD3, OLR-1 in response to stimulation of WT-PPARγ by endogenous ligands, with T0070907 being the most potent. Again, this effect is overcome somewhat by troglitazone. Taken together, the data in FIGS. 14A and 14B show that activation of the Group 2 genes by PPARγ requires more potent ligands and is significantly more sensitive antagonists than the Group 1 genes:. We also considered it important to determine whether mutation of F372 had simply dampened the ligand binding affinity of PPARγ and, consequently, had shifted the dose response to troglitazone significantly to higher concentrations. To investigate this possibility, WT PPARγ and EF-PPARγ cells were induced to differentiate for 5 clays in the presence of increasing concentrations of troglitazone. At this stage, cells were harvested for analysis of select genes by RT-PCR. FIG. 14C shows abundant expression of select Group 1 genes including C/EBPα, adiponectin and FABP4/aP2 in WT-PPARγ cells due to endogenous ligands (lane 1) with no significant change in expression in response to increasing doses of troglitazone (lanes 2-8), As expected, the Group 2 genes, FGF21 and OLR-1, are not expressed in WT-PPARγ without an exogeneous ligand (lane 1), but can be induced in a troglitazone dose-dependent manner (lanes 2-8). Analysis of gene expression in the EF-PPARγ showed that the Group 1 genes, C/EBPα, adiponectin and FABP4/aP2, are not expressed in the absence of exogenous ligand (FIG. 14C, lane 9) but, as expected, are induced in response to doses of troglitazone (250-500 nM) previously shown to be specific for PPARγ (FIG. 14C, lanes 10-16). Of importance is the observation that expression of FGF21 and OLR1 cannot be activated in the EF-PPARγ by doses of troglitazone (10 μM) that far exceed the dose that is specific for PPARγ. These data demonstrate clearly that mutation of F372 within helix 7 has prevented PPARγ from responding to endogenous ligands, but additionally, has prevented PPARγ from inducing expression of the Group 2 in response to the thiazolidinedione, troglitazone.

PPARγ Directly Regulates Expression of FGF21

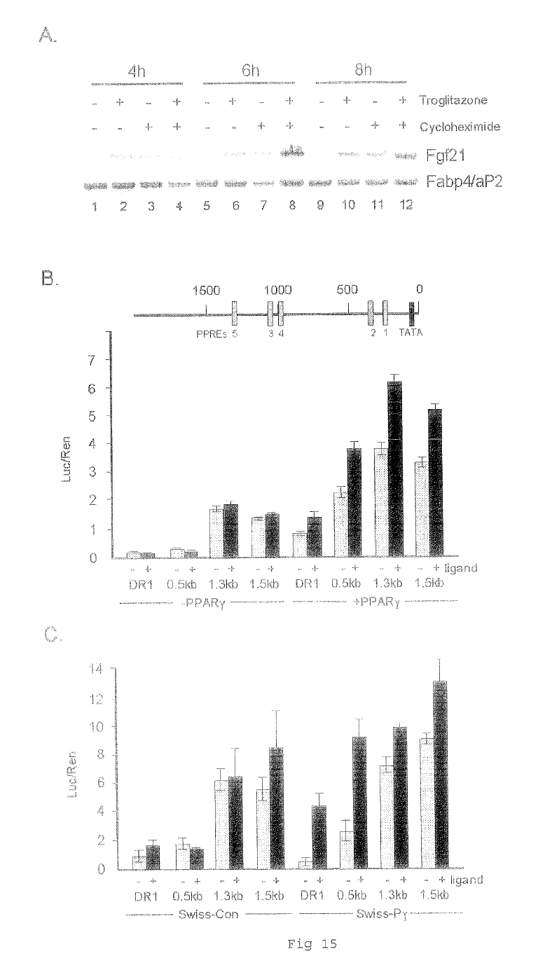

It is conceivable that the inability of the mutant PPARγ (EF or F) to induce expression FGF21 and OLR1 by troglitazone as well as exogenous ligands is because the corresponding genes might not be direct targets of PPARγ. Other studies, however, have shown a direct induction of the OLR1 gene promoter by PPARγ (5). We considered it important and of significant interest to determine whether the FGF21 gene is also a direct target of PPARγ. To this end, we performed two sets of experiments. First, we determined whether the induction of FGF21 gene expression MOH, in the absence of ongoing protein synthesis. To do this, WT-PPARγ cells were induced to differentiate for 5 days without a synthetic ligand at which stage either troglitazone (5 μM) or cycloheximide (5 μg/ml) was added alone or together for 4, 6 or 8 hrs and at each time RNA was analyzed by RT-PCR analysis. FIG. 15A shows significant expression of FABP4/aP2a mRNA at all three times due to its activation by the endogeneous ligand activity during the 5 days of differentiation of the WT-PPARγ cells. In contrast, there is virtually undetectable levels of FGF21 mRNA expression in the absence of an exogenous ligand (FIG. 15A, lanes 1, 5 and 9). Interestingly, exposure to troglitazone rapidly induces FGF21 mRNA expression during the 8 hr exposure time (FIG. 15A, lanes 2, 6 and 10) and this event occurs in the presence of cycloheximide (FIG. 15A, lanes 4, 8 and 12) showing that the FGF21 gene is a direct target of PPARγ. Also of interest is the observation that FGF21 mRNA expression is induced simply due to exposure to cycloheximide (FIG. 15A, lanes 3, 7 and 11). This is usually indicative of the existence of a repressor that is removed due to its rapid turnover in the absence of ongoing protein synthesis. To demonstrate further that PPARγ directly activates FGF21 gene expression, we performed a series of FGF21 gene promoter/luciferase reporter assays. To this end, fragments (−500, −1300 and −1500) of the upstream region of FGF21 gene were cloned into the pGL3 luciferase reporter plasmid as shown in FIG. 15B. Analysis of the sequence of the proximal 1500 by of the gene showed the presence of at least five DR-1 elements that are highly homologous to a consensus PPAR regulatory element (PPRE) and, therefore, have the potential to associate with PPARγ/RXRα heterodimers. FIG. 15B shows that transfection of the −500 bp fragment plasmid which contains two PPREs into control Swiss fibroblasts in the presence or absence of a potent PPARγ ligand, GW1929, expresses a low basal level of luciferase activity equivalent to a control DR-1/luciferase reporter composed of consensus PPREs. Interestingly, the 1300 bp and 1500 bp fragments express a higher level of luciferase activity, but the presence of GW1929 has no affect on this activity. Transfection of the reporter plasmids along with a PPARγ expression plasmid, however, resulted in a significant increase in the activity of all three FGF21 gene fragments, which was enhanced even further in the presence of GW1929, We also analyzed FGF21 promoter activity in control (pBabe-puro) and Swiss-WT-PPARγ cells by transfecting each of the luciferase reporter plasmids in the presence or absence of GW1929. The results in FIG. 5C are consistent with those in FIG. 15B showing that the transcriptional activity of the 500 bp fragment of the FGF21 gene is significantly higher in the Swiss cells expressing PPARγ compared to control Swiss cells and, that this activity is enhanced many fold by GW 1929. The activity of the 1300 and 1500 by fragments in WT-PPARγ cells in the presence of GW1929 is only slightly higher than the 500 by region suggesting that elements within this proximal promoter are likely responsible for the observed PPARγ-dependent activity of all three fragments.

Expression of many of the Group 2 Genes are Actively Repressed by SIRT1 During Terminal Adipogenesis

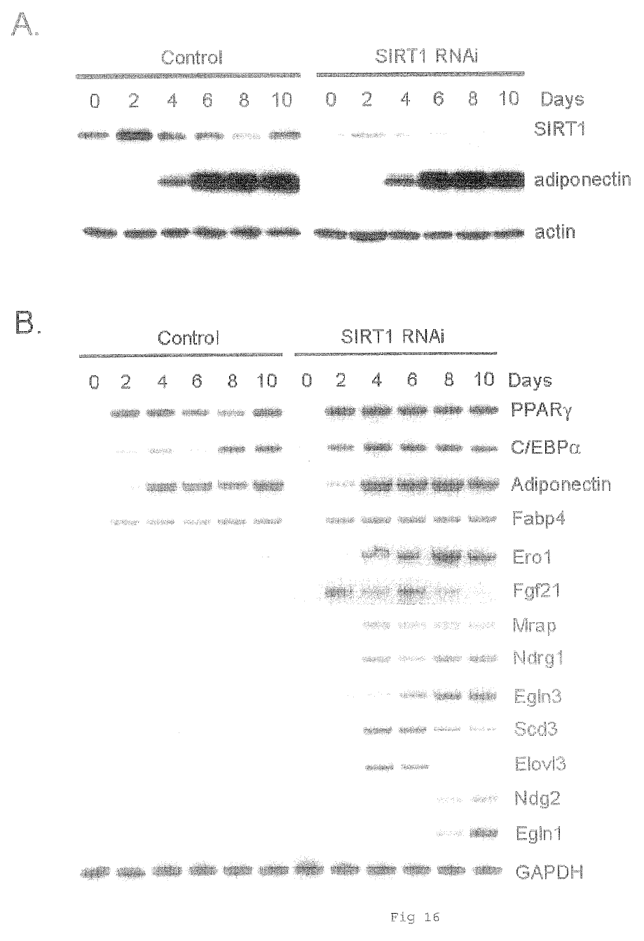

A recent report demonstrated that activation of SIRT1 in adipocytes triggers lipolysis and loss of fat by mechanisms involving repression of PPARγ activity (28). We questioned, therefore, whether there is a role for SIRT1 in facilitating the differential expression of the Group 1 and Group 2 genes as preadipocytes become mature fat cells. Consequently, we analyzed expression of adipogenic genes during differentiation of 3T3-L1 preadipocytes in which SIRT1 expression is suppressed due to constitutive production of a corresponding SIRT1 RNAi. The western blot in FIG. 16A shows the extensive reduction in SIRT1 expression in the RNAi cells compared to the abundant production in a control line of 3T3-L1 cells expressing the vector alone. It is also relevant that expression of SIRT1 increases several-fold during the early phase of adipogenesis in the control cells, but then subsides to preadipocyte levels during the terminal phase. It is also important to point out that there is no significant effect of knockdown of SIRT 1 on production of adiponectin. Total RNA was harvested from SIRT1. knockdown as well as control cells at select times throughout differentiation. RNA from preadipocytes (day 0), cells at a mid (4 days) and late phase (day 10) of adipogenesis was subjected to oligonucleotide microarray analysis employing affymetrix chips as discussed above. The relative expression of select mRNAs corresponding to both Group 1 and Group 2 genes was analyzed as shown in Tables 1A and 1B, respectively. As discussed above, Table 1A corresponds to a list of classic adipogenic genes (Group 1) that are induced during adipogenesis it, 3T3-L1 preadipocytes and are differentially responsive to WT-PPARγ versus EF-PPARγ, Suppression of SIRT1 activity causes a transient increase (˜50%) in expression of most of these genes at day 4 of differentiation in 3T3-L1 cells compared to their level of expression at this stage of differentiation in control cells (Table 1A, compare column 9 with 6). This difference in expression correlates with a significant increase in SIRT1 expression during early adipogenesis in control 3T3-L1 preadipocytes (FIG. 16A). Interestingly, these genes appear to reach a maximum level of expression by day 4 in the knockdown cells, whereas it requires 10 days for them to reach this maximum in, the control cells (Table 1A, compare columns 7 and 10). The data are consistent with the notion that the preadipocytes lacking SIRT1 activity differentiate much faster than control cells reaching terminal adipogenesis within 4 days, compared to 10 days in the controls cells. Table 1B shows the expression profiles of the Group 2 mRNAs in control and SIRT1 knockdown 3T3-L1 cells and Swiss-Pγ cells. As observed for the Group 1 genes in Table 1A, expression of the Group 2 genes also increases at day 4 of differentiation in the knockdown cells even though most of the genes do not normally show an enhanced expression at this stage of differentiation in control cells (Table 1B, compare column 9 with column 6). More important, most of the Group 2 genes are expressed at significantly higher levels at day 10 in the SIRT1 knockdown cells compared to control cells (Table 1B, compare column 10 with column 7). In fact, some genes most notably Ero1-Lα (487%), Hig1 (262%) and Trib3 (290%) are enhanced many fold in response to reduction in SIRT1 abundance. Additionally, all the genes coding for glycolytic enzymes as well as the glucose transporter 1 are also induced in the SIRT1 knockdown cells. It is also worth mentioning that the extent of induction of each of these Group 2 genes appears to correlate with their level of induction by troglitazone in WT-PPARγ cells (Table 1B, compare columns 1 and 2 with 7 and 10, respectively). To confirm the data presented in Tables 1A and 1B, FIG. 16B shows an RT-PCR analysis of RNA harvested from control and SIRT1 knockdown 3T3-L1 cells at times throughout differentiation. It is quite apparent that the knockdown of SIRT1 has a selective effect on the Group 2 genes compared to the Group 1 genes. Specifically, expression of C/EBPα and adiponectin mRNAs (Group 1) shows a modest increase at the early stage of adipogenesis in the SIRT1 knockdown cells (FIG. 16B), as presented in Table 1A, but no significant increase as these cells mature into adipocytes. In contrast, expression Group 2 genes including Ero1-Lα, Scd3, 21 and Elovl3 is dramatically enhanced in the SIRT1 knockdown cells (FIG. 16B).

Group 2 Genes are Selectively Induced in Mature Adipocytes by Exposure to PPARγ Ligands

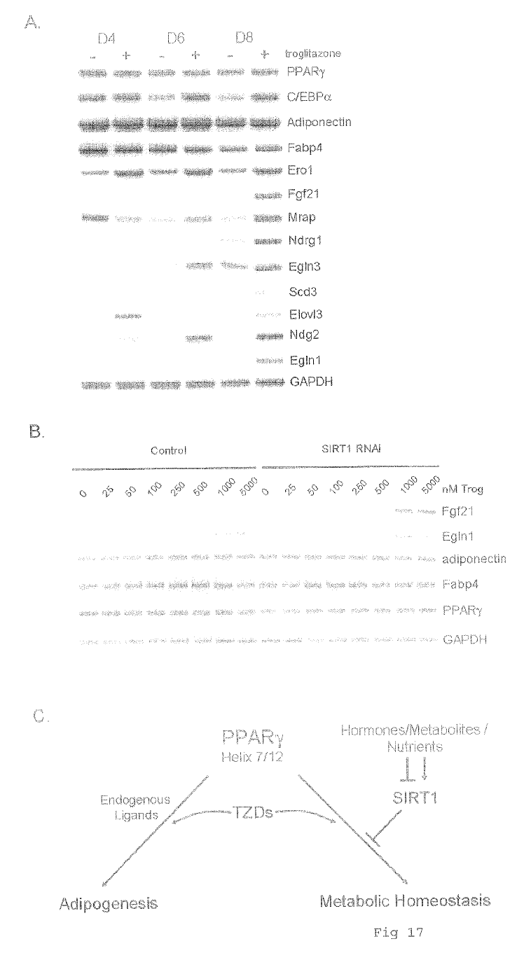

The data presented in FIG. 16B and Table 1B suggested that several of the Group 2 genes are actively repressed in mature adipocytes by mechanisms involving SIR1. We questioned, therefore, whether exposure of such cells to a synthetic PPARγ ligand could overcome the repression and stimulate their expression. To test this notion, normal 3T3-L1 preadipocytes were induced to differentiate following standard procedures and at days 2, 4 and 6 differentiating cells were exposed to troglitazone for 2 days at which time total RNA was harvested for analysis employing RT-PCR. FIG. 17A demonstrates an extensive induction of selected Group 2 genes at different stages of the differentiation process. Specifically, ELOVL3 is induced by exposure of the 3T3-L1 cells to troglitazone as early as 4 days of adipogenesis and corresponding mRNAs levels remain elevated throughout differentiation. FGF21 and Ero1-Lα gene expression is also enhanced several fold but only occurs in more mature adipocytes. Expression of the selected members of the Group 1 genes (C/EBPα, adiponectin and FABP4), however, are essentially unresponsive to the exogenous ligand since the level of expression is already at a maximum due to their induction by endogenous PPARγ ligands. These data are consistent with the hypothesis that a subset of the Group 2 genes including FGF21 and Ero1-Lα are actively repressed by SIRT1 in mature adipocytes and that this repression can be overcome by exposure to troglitazone. The fact that attenuation of SIRT1 (FIG. 16B) or exposure to the PPARγ ligand (FIG. 17A) does not induce expression of the classic adipogenic genes in Group 1 suggest that they are distinct from the Group 2 genes since they are presumably in a constant state of optimum transcriptional activity. These data suggest that SIRT1 and PPARγ ligands reciprocally regulate PPARγ activity on Group 2 genes; we questioned, therefore, whether SIRT1 might attenuate the response of PPARγ to its ligands. To test this notion, we determined the dose of troglitazone required to induce expression of select Group 2 genes in control versus SIRT1 knockdown 3T3-L1 preadipocytes4 days of differentiation. Specifically, differentiating to increasing doses of troglitazone for 2 days at which stage RNA was analyzed for expression of select genes by RT-PCR, FIG. 17B shows that expression of FGF21 and Egln1 (Group 2 genes) is induced in control cells following exposure to doses of troglitazone in range of I to 5 μM; in contrast, induction of these genes in SIRT1 knockdown cells requires a significantly lower dose of troglitazone (250 nM). These data suggest that SIRT1 attenuates the response of PPARγ to an exogenous ligand. Additionally, FIG. 17B also confirms that there is a negligible effect of either knockdown of SIRT1 or ligands on expression of the Group I genes adiponectin or FABP4.

TABLE 1A

| | Swiss-PPARγ Fibroblasts | | | | 3T3-L1 Preadipocytes | | | | | |
|---|---|---|---|---|---|---|---|---|---|---|
| Gene | WT − T | WT + T | EF − T | EF + T | C0 | C4 | C10 | Si0 | Si4 | Si10 |
| AdipoQ | 31337 | 21800 | 2 | 16173 | 22 | 11485 | 21530 | 29 | 18125 | 19233 |
| Cfd (adipsin) | 29067 | 14007 | 16 | 21924 | 17 | 7403 | 19353 | 13 | 13913 | 19063 |
| Fabp4 | 28044 | 34094 | 99 | 27865 | 2548 | 15883 | 21992 | 1649 | 22476 | 19072 |
| Ipl | 27508 | 30981 | 46 | 10384 | 5877 | 10129 | 15478 | 5659 | 14750 | 13787 |
| Mgst1 | 21264 | 25902 | 296 | 21101 | 9140 | 11735 | 15265 | 9292 | 14703 | 13179 |
| Fsp27 | 17352 | 18491 | 1 | 4663 | 21 | 5048 | 7758 | 3 | 10488 | 6880 |
| Fabp5 | 14193 | 17743 | 1198 | 12025 | 1514 | 9556 | 8836 | 832 | 15567 | 6537 |
| Hsd11b1 | 13323 | 5095 | 135 | 4034 | 221 | 1002 | 4712 | 274 | 1503 | 4886 |
| Mgst3 | 12555 | 19278 | 771 | 12997 | 1471 | 9479 | 7164 | 1563 | 11710 | 6868 |
| Cd36 | 9442 | 15517 | 4 | 2730 | 42 | 3556 | 5677 | 21 | 4712 | 4159 |
| Pnpla2 | 9415 | 10278 | 131 | 5568 | 263 | 5095 | 5085 | 345 | 8289 | 5496 |
| S3-12 | 9354 | 8558 | 362 | 10917 | 615 | 2427 | 3222 | 919 | 3768 | 3708 |
| Retn | 9271 | 4153 | 8 | 2182 | 5 | 2644 | 9900 | 28 | 8014 | 8279 |
| Acsl1 | 8896 | 15365 | 288 | 9153 | 463 | 9514 | 12690 | 376 | 14750 | 10720 |
| Orm1 | 8266 | 2249 | 753 | 4663 | 39 | 2461 | 3369 | 40 | 4233 | 2793 |
| Dgat1 | 6657 | 6429 | 477 | 3496 | 300 | 2543 | 2497 | 482 | 4315 | 2888 |
| Crat | 4095 | 5527 | 291 | 3479 | 440 | 1027 | 1092 | 387 | 2186 | 1925 |
| Slc25a10 | 3536 | 3762 | 196 | 1606 | 449 | 2633 | 3466 | 644 | 4272 | 3631 |
| Glul | 3386 | 1083 | 224 | 1208 | 688 | 8006 | 5884 | 945 | 9291 | 5250 |
| Cebpa | 2212 | 1570 | 72 | 1357 | 50 | 822 | 1750 | 76 | 1898 | 1774 |
| Pex11a | 1791 | 2164 | 152 | 3449 | 367 | 1567 | 1513 | 466 | 1854 | 2640 |
| S100a1 | 1629 | 1985 | 99 | 1078 | 159 | 1759 | 3025 | 206 | 3092 | 3128 |
| Nr1h3 | 1471 | 1172 | 101 | 892 | 113 | 606 | 500 | 99 | 1111 | 504 |
| Adhfe1 | 1340 | 982 | 10 | 518 | 248 | 1535 | 3662 | 237 | 2486 | 3948 |
| Ppargc1b | 1111 | 1362 | 150 | 745 | 72 | 875 | 676 | 154 | 764 | 763 |
| Aldh1a7 | 865 | 1409 | 29 | 3264 | 96 | 4189 | 2509 | 90 | 7867 | 2236 |
| Lgals12 (galectin12) | 810 | 344 | 1 | 136 | 16 | 137 | 707 | 4 | 343 | 851 |
| Ephx2 | 355 | 4516 | 9 | 7296 | 692 | 870 | 1458 | 1380 | 779 | 1745 |
| Aqp7 | 183 | 227 | 3 | 137 | 19 | 59 | 182 | 7 | 47 | 116 |
| | 1 | 2 | 3 | 4 | 5 | 6 | 7 | 8 | 9 | 10 |

TABLE 1B

| | Swiss-PPARγ Fibroblasts | | | | 3T3-L1 Preadipocytes | | | | | |
|---|---|---|---|---|---|---|---|---|---|---|
| Gene | WT − T | WT + T | EF − T | EF + T | C0 | C4 | C10 | Si0 | Si4 | Si10 |
| Ero1l | 2674 | 11266 | 424 | 631 | 2586 | 1582 | 2193 | 2859 | 4133 | 12875 |
| Higd1a | 3745 | 5145 | 1159 | 1791 | 1808 | 6360 | 2824 | 1730 | 7653 | 10231 |
| Trib3 | 330 | 1430 | 198 | 176 | 1095 | 344 | 848 | 982 | 430 | 3315 |
| Hig2 | 339 | 1084 | 258 | 212 | 290 | 287 | 211 | 273 | 440 | 1424 |
| Ndrg1 | 321 | 1138 | 661 | 212 | 82 | 63 | 110 | 74 | 228 | 691 |
| Cbr1 | 269 | 552 | 33 | 3 | 812 | 1929 | 1637 | 905 | 3778 | 4401 |
| Egln3 | 235 | 1201 | 93 | 299 | 148 | 290 | 187 | 71 | 697 | 1762 |
| Ndg2 | 166 | 5676 | 42 | 121 | 56 | 25 | 39 | 69 | 31 | 301 |
| Mrap | 170 | 1052 | 3 | 7 | 9 | 860 | 1389 | 3 | 2296 | 1833 |
| Klf15 | 228 | 348 | 1 | 73 | 2 | 41 | 653 | 3 | 270 | 1364 |
| Pdxp | 195 | 382 | 97 | 97 | 26 | 138 | 192 | 78 | 357 | 612 |
| lgfbp4 | 209 | 297 | 8 | 60 | 1034 | 1149 | 2400 | 2004 | 1378 | 4019 |
| Cox7a1 | 46 | 229 | 18 | 16 | 22 | 27 | 153 | 39 | 181 | 641 |
| Klb | 33 | 150 | 23 | 19 | 5 | 59 | 312 | 44 | 238 | 475 |
| Fgf21 | 8 | 760 | 4 | 43 | 28 | 6 | 7 | 7 | 7 | 17 |
| Gpi1 | 5844 | 10774 | 2457 | 3719 | 3814 | 3377 | 3393 | 3897 | 7071 | 10210 |
| Pfkp | 196 | 286 | 64 | 16 | 656 | 1088 | 726 | 420 | 1355 | 2263 |
| Aldo1 | 10441 | 16263 | 3066 | 6637 | 9384 | 8372 | 11841 | 9245 | 16551 | 16209 |
| Tpi1 | 6352 | 12858 | 3570 | 2843 | 7313 | 8203 | 9455 | 8923 | 16238 | 16001 |
| Gpd | 6559 | 12582 | 31 | 487 | 39 | 1298 | 3926 | 41 | 2917 | 2896 |
| Pgk1 | 9484 | 23358 | 9137 | 7203 | 10267 | 9535 | 9411 | 10097 | 17743 | 15875 |
| Eno1 | 10124 | 22186 | 9391 | 6322 | 12755 | 7873 | 9633 | 11881 | 14230 | 14547 |
| Gapdh | 24864 | 34531 | 15478 | 15634 | 15115 | 12861 | 16330 | 15979 | 20513 | 19695 |
| Gyk | 706 | 2098 | 221 | 944 | 26 | 75 | 46 | 23 | 67 | 63 |
| Slc2a1 | 527 | 1821 | 831 | 263 | 877 | 546 | 567 | 911 | 971 | 2106 |
| Scd3 | 518 | 1535 | 59 | 38 | 11 | 92 | 126 | 37 | 290 | 114 |
| Rbp4 | 335 | 481 | 2 | 4 | 16 | 34 | 22 | 50 | 32 | 32 |
| Olr1 | 163 | 1622 | 19 | 1 | 134 | 169 | 48 | 34 | 137 | 32 |
| Elovl3 | 38 | 1845 | 25 | 31 | 9 | 68 | 44 | 35 | 150 | 40 |
| Ehhadh | 27 | 780 | 3 | 16 | 1 | 2 | 50 | 1 | 13 | 35 |
| | 1 | 2 | 3 | 4 | 5 | 6 | 7 | 8 | 9 | 10 |

Discussion

Our recent studies have demonstrated that helix 7 within the ligand-binding domain of PPARγ facilitates a functional interaction between β-catenin and PPARγ (22). Additionally, other investigations by Glass and coworkers (26) identified K365 in helix 7 of PPARγ1 as a target for ligand dependent SUMOylation, which regulates the repression of inflammatory genes by PPARγ in macrophages. These observations suggested to us that helix 7 might also participate in ligand-dependent control of PPARγ target gene expression during adipogenesis. In the present studies, we show that ectopic expression of a WT-PPARγ in Swiss mouse fibroblasts induces expression of the adipogenic program in response to the normal cocktail of adipogenic inducers, including DEX, MIX, insulin and FBS, without the need for additional stimulation with an exogenous synthetic PPARγ ligand such as troglitazone. In fact, treatment with troglitazone appears to have only a minimal effect on the already robust expression of the adipogenic genes (FIG. 11 and Table 1A). In contrast, expression of mutant PPARγ, in which F372 within helix 7 of the ligand-binding domain has been changed to alanine, completely destroys the ability of PPARγ to induce adipogenesis in response to endogenous ligands (minus troglitazone) Interestingly, F3 72A-PPARγ can respond to troglitazone, and in doing so, activates expression of many of the genes induced by the WT-PPARγ; although, a subset of these genes are unresponsive to troglitazone-activated, F3 722-PPARγ (FIG. 12A and Table 1B, Group 2 genes) subset consists of a diverse group of genes encoding a novel set of adipocyte proteins such as Ero1-Lα and FGF21 as well as components of the glycolytic pathway and regulators of glucose uptake. Many of these Group 2 genes are constitutively produced at a low level during adipogenesis, but their expression can be mature adipocytes by exposure to potent PPARγ ligands or suppress SIRT1 activity (FIGS. 16 and 17). The studies also show that PPARγ directly regulates expression of the FGF21 gene through elements located within the 500 bp upstream region of the gene (FIG. 15) Taken together, these data are consistent with the notion that PPARγ can differentially regulate multiple programs of gene expression in response to ligands activating different regions of the ligand-binding domain. Moreover, activation of the Group 2 genes by PPARγ requires its association with a potent ligand to overcome the selective, suppressive effects of SIRT1.

The molecular mechanisms responsible for distinguishing one set of target genes from another likely involves recruitment of different coregulators to PPARγ docked on the promoters/enhancers of the genes. In fact, Lazar and coworkers have recently shown that GyK and OLR1 genes are actively repressed in mature adipocytes by recruitment of NCoR/HDAC3 complexes to PPARγ docked on PPAR response elements (PPRE) in the promoters of the corresponding genes (5, 12). Interestingly, exposure of adipocytes to TZDs dislodges the repressor complexes from these sites by mechanisms involving PPARγ coactivator-1α (PGC-1α) leading to expression of the genes. These authors also demonstrate that other adipogenic genes, such as FABP4/aP2, that are abundantly expressed in mature adipocytes in response to endogenous ligand activity, have PPARγ docked on their enhancers in association with the SRC/p160 family of coactivators. The data presented here are consistent with these observations, but also suggest that a subset of Group 2 genes including Ero1-Lα and FGF21 are selectively repressed by mechanisms dependent on SIRT1 activity. A mechanism under consideration involves a regulated SUMOylation of K365 within helix 7 of PPARγ that is docked on the specific set of target genes (Group 2) destined for suppression by SIRT1 in mature adipocytes. SUMOylated PPARγ will then recruit select corepressors such as NCoR/HDAC3 as well as SIRT1 to the target genes that are then subsequently repressed. PPARγ, which is docked on the genes (Group 1) that remain active during this process, likely escapes SUMOylation. Formulation of this model is based on the recent findings of Glass and coworkers, which demonstrated that ligand-dependent SUMOylation of PPARγ on K365 of helix 7 induces the PPARγ-associated repression of inflammatory genes in macrophages (26). K365 could also be a target of acetylation in which case acetylated K365 would prevent SUMOylation and, consequently, maintain PPARγ in active state, follows, therefore, that deacetylation of K365 by SIRT1 should facilitate SUMOylation resulting in repression of PPARγ on select target genes. An important question in considering this model is by what means does the SUMOylation process select PPARγ molecules that are docked on the targets that will be repressed during terminal adipogenesis? One possibility is that the environment surrounding the PPREs within these genes facilitates SUMOylation. For instance, the mechanism could involve docking of other nuclear factors that are induced during adipogenesis to sites that are flanking the PPREs. These factors could then participate in recruitment of the SUMOylation machinery to PPARγ and the resulting repression of these genes.

It is also possible that SIRT1 regulates the expression/activity of coregulators whose association with PPARγ is dependent on helix 7 of the ligand-binding domain. In fact, it is reasonable to suggest a role for PGC-1α since Lazar and coworkers have previously shown induction of this coactivator in white adipocytes in response to TZDs (12). Furthermore, these investigators demonstrated the involvement of PGC-1α in the selective activation of the Gyk gene by TZDs. Additionally, other studies have shown that SIRT1 deacetylates PGC-1α and in doing so regulates its ability to modulate the activity of different transcription factors (31). Consequently, it is conceivable that the selective expression of the Group 2 genes that includes Gyk and OLR1 in mature adipocytes involves induction of PGC-1α by TZDs and its activation through the suppression of SIRT1 activity.

Another important component of the model explaining how PPARγ activates different programs of gene expression at precise times during adipogenesis is the role played by specific ligands. As stated earlier, the endogenous ligands responsible for stimulating PPARγ activity during adipogenesis have not as yet been identified. Several studies have, however, identified signaling pathways and transcription factors that appear to regulate ligand production (4, 14, 17, 23, 35). The combined data are consistent with a regulated process induced during the initial days of adipogenesis involving cAMP signaling and enzymes that convert polyunsaturated fatty acids into eicosinoids. It is interesting that ligand activity peaks at 2-4 days of adipogenesis in 3T3-L1 preadipocytes but then rapidly subsides during terminal adipogenesis. Several questions result from these observations; most notably, how does PPARγ continue to maintain expression of most target genes such as FABP4/aP2 in the presence of lower concentrations of these ligands? There are many explanations for this apparent conundrum, including changes in expression and activity of coregulators during adipogenesis that requires lower levels of PPARγ activity to facilitate the associated recruitment/dislodgment process.

The subset of adipogenic genes (Group 2) that have been identified in this report contains several members that have not previously been shown to be regulated during adipogenesis or responsive to the activity of PPARγ. In the case of the ER oxidoreductase, Ero1-Lα, studies have recently shown that this protein is involved in regulating the secretion of adiponectin from mature adipocytes (30, 36). Furthermore, expression of Ero1-Lα mediates the nutrient control of adiponectin secretion by responding to the activity of the NAD-dependent deacetylase SIRT1 (30); data that are consistent with the observations presented here showing that the Group 2 set of adipogenic genes is regulated by SIRT1. It is also noteworthy that we have identified FGF21 as a direct target of PPARγ since it has recently been shown to be a hormone produced in the liver in response to activation of PPARα and acts as a component of the body's adaptation to fasting (1, 15). Other studies have also shown that it is a potent regulator of glucose uptake in 3T3-L1 adipocytes and primary human adipocytes (16). Our data show that FGF21 is not only produced in hepatocytes, but can also be induced in 3T3-L1 adipocytes by exposure to potent PPARγ ligands or suppression of SIRT1 activity, suggesting that this secreted factor might act in both an autocrine as well as a paracrine fashion to regulate insulin-responsive glucose uptake in adipocytes. With regard to FGF21 signaling, the data in Table 1B shows that a gene (Klb-(βKlotho) coding for an important component of FGF21 receptor (FGFR1 and 4) complex (25) is also responsive to both PPARγ and SIRT1 activity categorizing it as a member of the Group 2 gene family. Additionally, components of the glycolytic pathway and regulators of glucose uptake are also members of this novel Group 2 set of adipocytes genes. In fact, studies by others (31), have shown an increase in expression of liver pyruvate kinase and glucokinase in response to knockdown of SIRT1 in hepatocytes. We propose (FIG. 17C), therefore, that SIRT1 can control metabolic homeostasis by regulating expression of the Group 2 subset of adipocyte genes in response to metabolic effectors resulting in production of multiple proteins including insulin sensitizers such as adiponectin (through Ero1-Lα) as well as intracellular regulators of glucose uptake/metabolism (i.e., FGF2 and (βKlotho). It appears that synthetic PPARγ ligands also target this gene program in adipocytes by selectively overcoming the suppressive effects of SIRT1 on these genes.

In conclusion, our studies have identified a novel regulatory region of the ligand-binding domain of PPARγ that facilitates the selective expression of different subgroups of adipocyte genes during the formation of mature fat cells. These findings should provide a greater understanding of the role of PPARγ and its ligands in regulating physiological functions of adipocytes, most notably insulin responsiveness and energy balance. Furthermore, the identification of novel genes that respond to SIRT1 as well as PPARγ activity, such as Ero1-Lα and FGF21, should provide additional targets for the development of effective therapeutics to combat obesity and its associated disorders.

### REFERENCES

1. Badman, M. K., P. Pissios, A. R. Kennedy, G. Koukos, J. S. Flier, and E. Maratos-Flier, 2007. Hepatic Fibroblast Growth Factor 21 Is Regulated by PPARalpha and Is a Key Mediator of Hepatic Lipid Metabolism in Ketotic States. Cell Metab 5:426-437.
2. Berger, J., M. Tanen, A. Elbrecht, A. Hermanowski-Vosatka, D. E. Moller, S. Wright, and R. Thieringer. 2001. Peroxisome proliferator-activated receptor-gamma ligands inhibit adipocyte 11beta -hydroxysteroid dehydrogenase type 1 expression and activity. Biol Chem 276:12629-35.
3. Castriota, G., G. M. Thompson, Y. Lin, P. E. Scherer, D. E. Molter, and J. P. Berger. 2006. Peroxisome proliferator-activated receptor gamma agonists inhibit adipocyte expression of alpha1-acid glycoprotein. Cell Biol Int. 31:586-591.
4. Cheung, K. J., L Tzameli, P. Pissios, I. Rovira, O. Gavrilova, T. Ohtsubu, Z. Chen, T. Finkel, J. S. Flier, and J. M. Friedman. 2007. Xanthine Oxidoreductase Is a Regulator of Adipogenesis and PPARgamma Activity. Cell. Metab 5:115-28.
5. Chui, P. C., H. P. Guan, M. Lehrke, and M. A. Lazar. 2005. PPARgamma regulates adipocyte cholesterol metabolism via oxidized LDL receptor 1, J Clin Invest 115:2244-56.
6. Evans, R. M., G. D. Barish, and V. X. Wang, 2004. PPARs and the complex journey to obesity. Nat Med 10:355-61,
7. Fajas, L., M. B. Debril, and J. Auwerx. 2001. PPAR gamma: an essential role in metabolic control. Nutr Metab Cardiovasc Dis 11:64-9.
8. Farmer, S. R. 2006. Transcriptional control of adipocyte formation. Cell Metab 4:263-73.
9. Fasshauer, M., J. Klein, K. M. Kriauciunas, Field, M. Benito, and C. R. Kahn. 2001. Essential role of insulin receptor substrate 1 in differentiation of brown adipocytes. Mol Cell Biol 21:319-29.
10. Fasshauer, M., J. Klein, K. Ueki, K. M. Krianciunas, M. Benito, M. F. White, and C. R. Kahn, 2000. Essential role of insulin receptor substrate-2 in insulin stimulation of Glut4 translocation and glucose uptake in brown adipocytes. J Biol Chem 275:25494-501,
11. Ge, K., M. Guermah, C. X. Yuan, M. Ito, A. E. Wallberg, B. M. Spiegelman, and R. G. Roeder. 2002. Transcription coactivator TRAP220 is required for PPAR gamma 2-stimulated adipogenesis. Nature 417:563-7.

12. Guan, H. P., T. Ishizuka, P. C. Chui, M. Lehrke, and M. A. Lazar. 2005. Corepressors selectively control the transcriptional activity of PPARgamma in adipocytes. Genes Dev 19:453-61.
13. Guan, H. P., Y. Li, M. V. Jensen, C. B. Newgard, C. M. Steppan, and M. A. Lazar. 2002. A futile metabolic cycle activated in adipocytes by antidiabetic agents. Nat Med 8:1122-8.
14. Hamm, J. K., B. H. Park, and S. R. Farmer. 2001. A role for C/EBPbeta in regulating peroxisome proliferator-activated receptor gamma activity during adipogenesis in 3T3-LI preadipocytes. J Biol Chem 276:18464-71.
15. Inagaki, T., P. Dutchak, G. Zhao, X. Ding, L. Gautron, V. Parameswara, Y. Li, R. Goetz, M. Mohammadi, V. Esser, J. K. Elmquist, R. D. Gerard, S. C. Burgess, R. E. Hammer, D. J. Mangelsdorf, and S. A. Kliewer. 2007. Endocrine Regulation of the Fasting Response by PPARalpha-Mediated Induction of Fibroblast Growth Factor 21. Cell Metab 5:415-25.
16. Kharitonenkov, A., T. L. Shiyanova, A. Koester, A. M. Ford, R. Micanovic, E. J. Galbreath, G. E. Sandusky, L. J. Hammond, J. S. Moyers, R. A. Owens, J. Gromada, J. T. Brozinick, E. D. Hawkins, V. J. Wroblewski, D. S. Li, F. Mehrbod, S. R. Jaskunas, and A. B. Shanafelt. 2005. FGF-2l as a novel metabolic regulator. J Clin Invest 115:1627-35.
17. Kim, J. B., H. M. Wright, M. Wright, and B. M. Spiegelman. 1998. ADD1/SREBP1 activates PPAR gamma through the production of endogenous ligand. Proc Natl Acad Sci U S A 95:4333-4337.
18. Kliewer, S. A., H. E. Xu, M. H. Lambert, and T. M. Wilson. 2001. Peroxisome proliferator-activated receptors: from genes to physiology. Recent Prog IIorm Res 56:239-63.
19. Krey, G., O. Braissant, F. L'Horset, E. Kalkhoven, M. Perroud, M. G. Parker, and W. Wahl. 1997. Fatty acids, eicosinoids, and hypolipidemic agents identified as ligands of peroxisome proliferator-activated receptors by coactivator-dependent receptor ligand assay. Mol Endocrinol 11:779-91.
20. Lehmann, J. M. L. B Moore, T. A. Smith-Oliver, W. O. Wilkison, T. M. Wilson, and S. A. Kliewer. 1995. An antidiabetic thiazolidinectione is a high affinity ligand for peroxisome proliferator-activated receptor gamma (PPAR gamma). 3. Biol, Chem, 270:12953-12956.
21. Lehrke, M., and M. A. Lazar. 2005. The many faces of PPARgamma. Cell 123:993-9.

22. Liu, J., H. Wang, Y. Zuo, and S. R. Farmer: 2006. Functional interaction between peroxisome proliferator-activated receptor gamma and beta-catenin. Mol Cell Biol 26:5827-37.
23. Madsen, L., R. K. Petersen, M. B. Sorensen, C. Jorgensen, P. Hallenborg, L. Pridal, J. Fleckner, E.-Z. Amri, P. Krieg, C. Furstenberger, R. K. Berge, and K. Kristiansen. 2003. Adipocyte differentiation of 3T3-L1 preadipocytes is dependent on lipoxygenase activity during the initial stages of the differentiation process. Biochem. J. 375:539-549.
24. Nolte, R, T., G. B. Wisely, S. Westin, J. E. Cobb, M. H. Lambert, R. Kurokawa, M. G. Rosenfeld, T. M. Wilson, C. K. Glass, and M. V. Milburn. 1998. Ligand binding and co-activator assembly of the peroxisome proliferator-activated receptor-gamma. Nature 395:137-43.
25. Ogawa, Y., H. Kurosu, M. Yamamoto, A. Nandi, K. P. Rosenblatt, R. Goetz, A. V. Eliseenkova, M. Mohammadi, and M. Kuro-o. 2007. BetaKlotho is required for metabolic activity of fibroblast growth factor 21. Proc Natl Acad Sci USA 104:7432-7.
26. Pascual, G., A. L. Fong, S. Ogawa, A. Gamliel, A. C. Li, V. Perissi, D. W. Rose, T. M. Willson, M. G. Rosenfeld, and C. K. Glass. 2005. A SUMOylation-dependent pathway mediates transrepression of inflammatory response genes by PPAR-gamma. Nature 437:759-63.
27. Perissi, V., A. Aggarwal, C. K. Glass, D. W. Rose, and M. G. Rosenfeld. 2004. A corepressor/coactivator exchange complex required for transcriptional activation by nuclear receptors and other regulated transcription factors. Cell 116:511-26.
28. Picard, F., M. Kurtev, N. Chung, A. Topark-Ngarm, T. Senawong, R. Machado De Oliveira, M. Leid, M. W. McBurney, and L. Guarente. 2004. Sirt1 promotes fat mobilization in white adipocytes by repressing PPAR-gamma. Nature 429:771-6.
29. Qi, C., S. Surapureddi, Y. J. Zhu, S. Yu, P. Kashireddy, M. S. R, and J. K. Reddy. 2003. Transcriptional coactivator PRIP, the peroxisome proliferator-activated receptor gamma (PPARgamma)-interacting protein, is required for PPARgamma-mediated adipogenesis J Biol Chem 278:25281-4.
30. Qiang, L., H. Wang, and S. R. Farmer. 2007. Adiponectin Secretion Is Regulated by SIRT1 and the Endoplasmic Reticulum Oxidoreductase Ero1-L {alpha} Mol Cell Biol 27:4698-707.
31. Rodgers, J. T., C. Lerin, W. Haas, S. P. Gygi, P. M. Spiegelman, and P. Puigserver. 2005. Nutrient control of glucose homeostasis through a complex of PGC-1alpha and SIRT1. Nature 434:113-8.
32. Rosen, E. D., and B. M. Spiegelman 2001. PPARgamma: a nuclear regulator of metabolism, differentiation, and cell growth. J Biol Chem 276:37731-4.
33. Schadinger, S. E., N. L. Bucher, B. M. Schreiber, and S. R. 1 2005. PPARgamma2 regulates lipogenesis and lipid accumulation in steatotic hepatocytes. Am J Physiol Endocrinol Metab 288:E1195-205.
34. Steppan, C. M., and M. A. Lazar. 2002. Resistin and obesity-associates insulin resistance. Trends Endocrinol Metab 13:18-23.
35. Tzameli, I., H. Fang, M. Ollero, H. Shi. J. K. Flamm, P. Kievit, A. N. Hollenberg, and J. S. Flier. 2004. Regulated production of a peroxisome proliferator-activated receptor-gamma ligand during an early phase of adipocyte differentiation in 3T3-L1 adipocytes. J Biol Chem 279:36093-102.
36. Wang, Z. V., T. D. Schraw, J. Y. Kim, T. Khan, M. W. Pajala, A. Follenzi, and P. E. Scherer. 2007. Secretion of the adipocyte-specific secretory protein adiponectin critically depends on thiol-mediated protein retention. Mol Cell Biol 27:3716-31.
37. Willson, T. M., M. H. Lambert, and S. A. Kliewer, 2001. Peroxisome proliferator-activated receptor gamma and metabolic disease. Annu Rev Biochem 70:341-67.

### Example 4

Fibroblasts that ectopically express PPARγ were treated with nicotinamide (Nico; positive control SIRT1 inhibitor), an active isomer of a SIRT1 inhibitor (E3); a racemic mixture of a SIRT1 inhibitor (E2); and an inactive isomer of a SIRT1 inhibitor (E1). (0) indicates that no SIRT1 inhibitor was added. The cells were then examined for levels of FGF21 expression in the presence or absence of troglitazone (Trog). Cells were differentiated for 4 days without the indicated compounds or troglitazone. On day 4, the compounds (E1, E2, E3, or nicotinamide), with or without troglitazone, were added for 2 days. Cells were harvested on day 6 for qPCR measurement of FGF21 mRNA. Three separate dishes of cells were analyzed for each condition.

The concentration used for E1, E2, E3, and troglitazone was 5 μM. Each of E1, E2, and E3 has a chemical purity of greater than or equal to 98%. The enantiomeric excess of El and E3 is 99%.

E3 is the (−) isomer of the following compound:

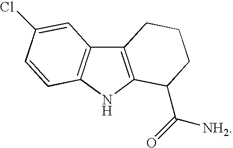

E2 is shown below:

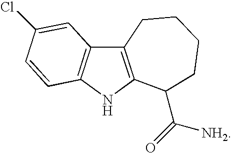

E1 is the (+) isomer of the following compound:

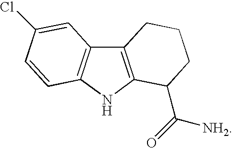

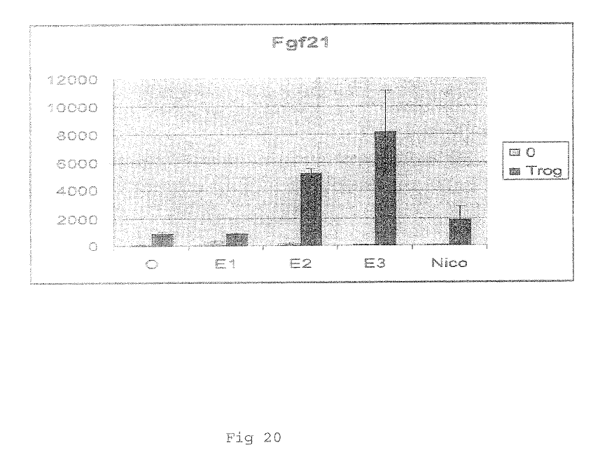

As shown in FIGS. 20, E2 and E3 potentiate the activity of troglitazone in inducing FGF21 expression.

What is claimed is:

1. A method of treating a disorder in a subject, the method comprising administering a SIRT1 inhibitor and a PPARγ agonist to the subject.

2. The method of claim 1, wherein the disorder is a metabolic disorder, a neoplastic disorder, dyslipidemia, arteriosclerosis, inflammation, a cardiovascular disorder, or ischemia.

3. The method of claim 1, wherein the SIRT1 inhibitor is a compound of formula (XI) below: 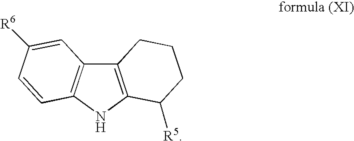
wherein R⁶ is halo or alkyl and wherein R⁵ is aminocarbonyl.

4. The method of claim 3, wherein the compound is 6-Chloro-2,3,4,9-tetrahydro-1H-carbazole-1-carboxylic acid amide.

5. The method claim 3, comprising a composition comprising at least a 60% enantiomeric excess of the enantiomer of formula (XI) having an optical rotation of −14.1 (c−0.33 DCM):

6. The method of claim 3, comprising a composition comprising at least a 60% enantiomeric excess of the enantiomer of formula (XI) having an optical rotation of −14.1 (c=0.33 DCM).

7. The method of claim 3, wherein the PPARγ agonist is a thiazolidinedione (TZD).

8. The method of claim 7, wherein the TZD comprises rosiglitazone, pioglitazone, troglitazone, or ciglitazone.

9. The method of claim 1, wherein the PPARγ agonist is a non-thiazolidinedione (non-TZD).

10. The method of claim 9, wherein the non-TZD comprises aleglitazar, muraglitazar, tesaglitazar, 15-deoxy-Δ_{12,14}-prostaglandin J₂ (15d-PGJ₂), GW1929, GW7845, RWJ-348260, AK109, mono-2-ethyhexyl phthalate, GI262570, an eicosanoid, or a tetrahydroisoquinoline PPARγ agonist.

11. A composition comprising a SIRT1 inhibitor and a PPARγ agonist.

12. The composition of claim 11, wherein the SIRT1 inhibitor is a compound of formula (XI) below: 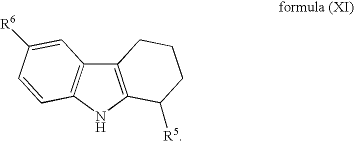
wherein R⁶ is halo alkyl and wherein R⁵ is aminocarbonyl.

13. The composition of claim 12, wherein the compound. 6-Chloro-2,3,4,9-tetrahydro-1H-carbazole-1-carboxylic acid amide.

14. The composition of claim 12, comprising a composition comprising at least a 60% enantiomeric excess of the enantiomer of formula (XI) having an optical rotation of −14A (c=0.33 DCM).

15. The composition of claim 12, comprising a composition comprising at least a 90% enantiomeric excess of the enantiomer of formula (XI) having an optical rotation of −14A (c=0.33 DCM).

16. The composition of claim 11, wherein the PPARγ agonist is a thiazolidinedione (TZD).

17. The composition of claim 16, wherein the TZD comprises rosiglitazone, pioglitazone, troglitazone, or ciglitazone.

18. The composition of claim 11, wherein the PPARγ agonist is a non-thiazolidinedione (non-TZD).

19. The composition of claim 18, wherein the non-TZD comprises aleglitazar, muraglitazar, tesaglitazar, 15-deoxy-Δ_{12,14}-prostaglandin J₂ (15d-PGJ₂), GW1929, GW7845, RWJ-348260, AK109, mono-2-ethyhexyl phthalate, GI262570, an eicosanoid, or a tetrahydroisoquinoline PPARγ agonist.


SEQUENCE LISTING (82 sequences)

SEQ ID NO: 1 - protein, 505 aa, Homo sapiens
MGETLGDSPIDPESDSFTDTLSANISQEMTMVDTEMPFWPTNFGISSVDLSVMEDHSHSFDIKPFTTVDFSSISTPHYEDIPFTRTDPVVADYKYDLKLQEYQSAIKVEPASPPYYSEKTQLYNKPHEEPSNSLMAIECRVCGDKASGFHYGVHACEGCKGFFRRTIRLKLIYDRCDLNCRIHKKSRNKCQYCRFQKCLAVGMSHNAIRFGRMPQAEKEKLLAEISSDIDQLNPESADLRALAKHLYDSYIKSFPLTKAKARAILTGKTTDKSPFVIYDMNSLMMGEDKIKFKHITPLQEQSKEVAIRIFQGCQFRSVEAVQEITEYAKSIPGFVNLDLNDQVTLLKYGVHEIIYTMLASLMNKDGVLISEGQGFMTREFLKSLRKPFGDFMEPKFEFAVKFNALELDDSDLAIFIAVIILSGDRPGLLNVKPIEDIQDNLLQALELQLKLNHPESSQLFAKLLQKMTDLRQIVTEHVQLLQVIKKTETDMSLHPLLQEIYKDLY

SEQ ID NO: 2 - DNA, 19 bp, Artificial Sequence
  Description of Artificial Sequence Synthetic primer
cttgtacgacgaagacgac

SEQ ID NO: 3 - DNA, 20 bp, Artificial Sequence
  Description of Artificial Sequence Synthetic primer
ggccacggataggtccatat

SEQ ID NO: 4 - DNA, 19 bp, Artificial Sequence
  Description of Artificial Sequence Synthetic primer
catagacacgctggaacag

SEQ ID NO: 5 - DNA, 13 bp, Artificial Sequence
  Description of Artificial Sequence Synthetic primer
agaccaaggagca

SEQ ID NO: 6 - DNA, 13 bp, Artificial Sequence
  Description of Artificial Sequence Synthetic primer
agacccaaggccc

SEQ ID NO: 7 - DNA, 13 bp, Artificial Sequence
  Description of Artificial Sequence Synthetic primer
tggcctgtggcca

SEQ ID NO: 8 - DNA, 14 bp, Artificial Sequence
  Description of Artificial Sequence Synthetic primer
tgagcacaaggccc

SEQ ID NO: 9 - DNA, 13 bp, Artificial Sequence
  Description of Artificial Sequence Synthetic primer
agttccagggcca

SEQ ID NO: 10 - DNA, 64 bp, Artificial Sequence
  Description of Artificial Sequence Synthetic primer
gatcccccttgtacgacgaagacgacttcaagagagtcgtcttcgtcgtacaagtttttggaaa

SEQ ID NO: 11 - DNA, 64 bp, Artificial Sequence
  Description of Artificial Sequence Synthetic primer
agcttttccaaaaacttgtacgacgaagacgactctcttgaagtcgtcttcgtcgtacaagggg

SEQ ID NO: 12 - DNA, 64 bp, Artificial Sequence
  Description of Artificial Sequence Synthetic primer
gatccccggccacggataggtccatattcaagagatatggacctatccgtggcctttttggaaa

SEQ ID NO: 13 - DNA, 64 bp, Artificial Sequence
  Description of Artificial Sequence Synthetic primer
agcttttccaaaaaggccacggataggtccatatctcttgaatatggacctatccgtggccggg

SEQ ID NO: 14 - DNA, 64 bp, Artificial Sequence
  Description of Artificial Sequence Synthetic primer
gatcccccatagacacgctggaacagttcaagagactgttccagcgtgtctatgtttttggaaa

SEQ ID NO: 15 - DNA, 64 bp, Artificial Sequence
  Description of Artificial Sequence Synthetic primer
agcttttccaaaaacatagacacgctggaacagtctcttgaactgttccagcgtgtctatgggg

SEQ ID NO: 16 - DNA, 36 bp, Artificial Sequence
  Description of Artificial Sequence Synthetic primer
gaaggatccatgggccgcgcctggggcttgctcgtt

SEQ ID NO: 17 - DNA, 37 bp, Artificial Sequence
  Description of Artificial Sequence Synthetic primer
cgccgtcgacggcacattccaaccgtcctcctcagtg

SEQ ID NO: 18 - RNA, 25 nt, Artificial Sequence
  Description of Artificial Sequence Synthetic oligonucleotide
gcugaguauguggacuuacuccuua

SEQ ID NO: 19 - RNA, 25 nt, Artificial Sequence
  Description of Artificial Sequence Synthetic oligonucleotide
gggcacugcucugaagaucuuguuu

SEQ ID NO: 20 - RNA, 25 nt, Artificial Sequence
  Description of Artificial Sequence Synthetic oligonucleotide
gggcucucuccaaagugcuuccauu

SEQ ID NO: 21 - DNA, 22 bp, Artificial Sequence
  Description of Artificial Sequence Synthetic primer
ccagagcatggtgccttcgctg

SEQ ID NO: 22 - DNA, 21 bp, Artificial Sequence
  Description of Artificial Sequence Synthetic primer
gagctgacccaatggttgctg

SEQ ID NO: 23 - DNA, 20 bp, Artificial Sequence
  Description of Artificial Sequence Synthetic primer
actcctggagagaaggagaa

SEQ ID NO: 24 - DNA, 24 bp, Artificial Sequence
  Description of Artificial Sequence Synthetic primer
ttgtccttcttgaagaggctcacc

SEQ ID NO: 25 - DNA, 21 bp, Artificial Sequence
  Description of Artificial Sequence Synthetic primer
gagcatggtgccttcgctgat

SEQ ID NO: 26 - DNA, 21 bp, Artificial Sequence
  Description of Artificial Sequence Synthetic primer
caaccattgggtcagctcttg

SEQ ID NO: 27 - DNA, 21 bp, Artificial Sequence
  Description of Artificial Sequence Synthetic primer
aaggtgctggagttgaccagt

SEQ ID NO: 28 - DNA, 21 bp, Artificial Sequence
  Description of Artificial Sequence Synthetic primer
tagagatccagcgacccgaaa

SEQ ID NO: 29 - DNA, 20 bp, Artificial Sequence
  Description of Artificial Sequence Synthetic primer
gtcaacagcaaaagccacaa

SEQ ID NO: 30 - DNA, 20 bp, Artificial Sequence
  Description of Artificial Sequence Synthetic primer
tctggggtcagaggaagaga

SEQ ID NO: 31 - DNA, 20 bp, Artificial Sequence
  Description of Artificial Sequence Synthetic primer
ccgacttttcagagccactc

SEQ ID NO: 32 - DNA, 19 bp, Artificial Sequence
  Description of Artificial Sequence Synthetic primer
cggttaggttggctaatgt

SEQ ID NO: 33 - DNA, 25 bp, Artificial Sequence
  Description of Artificial Sequence Synthetic primer
gaaggatccatgggccgcgcctggg

SEQ ID NO: 34 - DNA, 25 bp, Artificial Sequence
  Description of Artificial Sequence Synthetic primer
cgccgtcgacggcacattccaaccg

SEQ ID NO: 35 - DNA, 20 bp, Artificial Sequence
  Description of Artificial Sequence Synthetic primer
gtcatcctctgcctggtgtt

SEQ ID NO: 36 - DNA, 20 bp, Artificial Sequence
  Description of Artificial Sequence Synthetic primer
ttcttccgatgcaatccaat

SEQ ID NO: 37 - DNA, 20 bp, Artificial Sequence
  Description of Artificial Sequence Synthetic primer
tcgtctgcaaaatcgaaatg

SEQ ID NO: 38 - DNA, 20 bp, Artificial Sequence
  Description of Artificial Sequence Synthetic primer
gggaaaccatacagggaggt

SEQ ID NO: 39 - DNA, 20 bp, Artificial Sequence
  Description of Artificial Sequence Synthetic primer
ctgggggtctaccaagcata

SEQ ID NO: 40 - DNA, 20 bp, Artificial Sequence
  Description of Artificial Sequence Synthetic primer
aaggctctaccatgctcagg

SEQ ID NO: 41 - DNA, 20 bp, Artificial Sequence
  Description of Artificial Sequence Synthetic primer
atgaggtgttgatggccttt

SEQ ID NO: 42 - DNA, 20 bp, Artificial Sequence
  Description of Artificial Sequence Synthetic primer
ggtttcccataggtgtgtgc

SEQ ID NO: 43 - DNA, 20 bp, Artificial Sequence
  Description of Artificial Sequence Synthetic primer
cctgattctgcaacggagtt

SEQ ID NO: 44 - DNA, 20 bp, Artificial Sequence
  Description of Artificial Sequence Synthetic primer
ggctcaccagcttcattagc

SEQ ID NO: 45 - DNA, 20 bp, Artificial Sequence
  Description of Artificial Sequence Synthetic primer
cctggcactgcctagagact

SEQ ID NO: 46 - DNA, 20 bp, Artificial Sequence
  Description of Artificial Sequence Synthetic primer
gctttatgcacagcgatgaa

SEQ ID NO: 47 - DNA, 20 bp, Artificial Sequence
  Description of Artificial Sequence Synthetic primer
tgctgcaagaagagatgacg

SEQ ID NO: 48 - DNA, 20 bp, Artificial Sequence
  Description of Artificial Sequence Synthetic primer
ctcttgtgactcccgtctcc

SEQ ID NO: 49 - DNA, 20 bp, Artificial Sequence
  Description of Artificial Sequence Synthetic primer
gttgcaagctctcctgttcc

SEQ ID NO: 50 - DNA, 20 bp, Artificial Sequence
  Description of Artificial Sequence Synthetic primer
cagacttggtctcccacctc

SEQ ID NO: 51 - DNA, 20 bp, Artificial Sequence
  Description of Artificial Sequence Synthetic primer
ccgaaggaaattctgcagtc

SEQ ID NO: 52 - DNA, 20 bp, Artificial Sequence
  Description of Artificial Sequence Synthetic primer
cgcctagtgcagttgtttca

SEQ ID NO: 53 - DNA, 20 bp, Artificial Sequence
  Description of Artificial Sequence Synthetic primer
aatgtgtgatgcctttgtgg

SEQ ID NO: 54 - DNA, 20 bp, Artificial Sequence
  Description of Artificial Sequence Synthetic primer
aatttccatccaggcctctt

SEQ ID NO: 55 - DNA, 20 bp, Artificial Sequence
  Description of Artificial Sequence Synthetic primer
cgagagctacatgacgtgga

SEQ ID NO: 56 - DNA, 20 bp, Artificial Sequence
  Description of Artificial Sequence Synthetic primer
ctggcagaaggcatgtatcc

SEQ ID NO: 57 - DNA, 20 bp, Artificial Sequence
  Description of Artificial Sequence Synthetic primer
gagatgcctctgggacacat

SEQ ID NO: 58 - DNA, 20 bp, Artificial Sequence
  Description of Artificial Sequence Synthetic primer
ttctgccctttcttcagcat

SEQ ID NO: 59 - DNA, 20 bp, Artificial Sequence
  Description of Artificial Sequence Synthetic primer
agccatggttgcttgttacc

SEQ ID NO: 60 - DNA, 20 bp, Artificial Sequence
  Description of Artificial Sequence Synthetic primer
tgtccacgagtccacagaag

SEQ ID NO: 61 - DNA, 20 bp, Artificial Sequence
  Description of Artificial Sequence Synthetic primer
gaccggtgctaacaaaggaa

SEQ ID NO: 62 - DNA, 20 bp, Artificial Sequence
  Description of Artificial Sequence Synthetic primer
gctccttcttctgggctttt

SEQ ID NO: 63 - DNA, 20 bp, Artificial Sequence
  Description of Artificial Sequence Synthetic primer
cttcgtggtgctcctctttc

SEQ ID NO: 64 - DNA, 20 bp, Artificial Sequence
  Description of Artificial Sequence Synthetic primer
gacctggtcctaggggagag

SEQ ID NO: 65 - DNA, 20 bp, Artificial Sequence
  Description of Artificial Sequence Synthetic primer
gatgccaagtgtccagtcct

SEQ ID NO: 66 - DNA, 20 bp, Artificial Sequence
  Description of Artificial Sequence Synthetic primer
tctcccttcggtcagactgt

SEQ ID NO: 67 - DNA, 20 bp, Artificial Sequence
  Description of Artificial Sequence Synthetic primer
cgggtcttatggctcagatg

SEQ ID NO: 68 - DNA, 20 bp, Artificial Sequence
  Description of Artificial Sequence Synthetic primer
gaagccctcacacagggtta

SEQ ID NO: 69 - DNA, 20 bp, Artificial Sequence
  Description of Artificial Sequence Synthetic primer
gctctggtccggtcttttag

SEQ ID NO: 70 - DNA, 20 bp, Artificial Sequence
  Description of Artificial Sequence Synthetic primer
ccagcccaagcagtataagc

SEQ ID NO: 71 - DNA, 24 bp, Artificial Sequence
  Description of Artificial Sequence Synthetic primer
tgatgtgcagagtgtagtgcctca

SEQ ID NO: 72 - DNA, 24 bp, Artificial Sequence
  Description of Artificial Sequence Synthetic primer
acgtaaccacaccttcgactgcat

SEQ ID NO: 73 - DNA, 20 bp, Artificial Sequence
  Description of Artificial Sequence Synthetic primer
cagaaatgctccagggaaag

SEQ ID NO: 74 - DNA, 20 bp, Artificial Sequence
  Description of Artificial Sequence Synthetic primer
gatcttccttcctgggttcc

SEQ ID NO: 75 - DNA, 24 bp, Artificial Sequence
  Description of Artificial Sequence Synthetic primer
tcttctcagccggagtttcagctt

SEQ ID NO: 76 - DNA, 24 bp, Artificial Sequence
  Description of Artificial Sequence Synthetic primer
tgttgacaagctttctgtggtggc

SEQ ID NO: 77 - DNA, 21 bp, Artificial Sequence
  Description of Artificial Sequence Synthetic primer
atcaccatcttccaggagcga

SEQ ID NO: 78 - DNA, 22 bp, Artificial Sequence
  Description of Artificial Sequence Synthetic primer
gttgtcatggatgaccttggcc

SEQ ID NO: 79 - DNA, 18 bp, Artificial Sequence
  Description of Artificial Sequence Synthetic primer
aagcctcaccttgacacc

SEQ ID NO: 80 - DNA, 18 bp, Artificial Sequence
  Description of Artificial Sequence Synthetic primer
caggaaacaacccagctc

SEQ ID NO: 81 - DNA, 18 bp, Artificial Sequence
  Description of Artificial Sequence Synthetic primer
agtgcagacaagtcccct

SEQ ID NO: 82 - DNA, 18 bp, Artificial Sequence
  Description of Artificial Sequence Synthetic primer
ggcagctggaattgtgtt